(19) United States
(12) Patent Application Publication
(10) Pub. No.: US 2012/0322150 A1
(43) Pub. Date: Dec. 20, 2012

(54) Mechanism and Method for Regulating Glycogen Synthase Kinase 3 (GSK3)-Related Kinases

(75) Inventors: Tae-Wuk Kim, Washington, DC (US); Zhiyong Wang, Washington, DC (US)

(73) Assignee: CARNEGIE INSTITUTION OF WASHINGTON, WASHINGTON, DC (US)

(21) Appl. No.: 13/384,558

(22) PCT Filed: Jul. 16, 2010
(86) PCT No.: PCT/US2010/042265

§ 371 Date: Sep. 4, 2012

Related U.S. Application Data
(60) Provisional application No. 61/226,522, filed on Jul. 17, 2009.

(51) Int. Cl. C12N 5/071; C12N 5/04

(52) U.S. Cl. 435/366; 435/375; 435/410

(57) ABSTRACT

The present invention relates to a novel mechanism for regulating GSK3 kinases, including BIN2 and human GSK3-beta, by dephosphorylating GSK3 kinases through the PP1 phosphatase, such as the plant BSU1 phosphatases and human PP1-gamma.

### RELATED APPLICATIONS

The present application claims priority to U.S. Provisional Application No. 61/226,552, filed Jul. 17, 2009, which is hereby incorporated in its entirety.

### FIELD OF THE INVENTION

The present invention relates to the use of phosphatase activity to regulate protein kinases. The present invention relates to regulating the glycogen synthase kinases (GSKs) related kinases.

### Sequence Listing

A computer readable text file, entitled “056100-5081-WO-SeqListing.txt”, created on or about Jul. 14, 2010, with a file sixe of about 45 kb contains the ssequence listing for this application and is hereby incorporated by reference in its entirety.

### BACKGROUND OF THE INVENTION

The ability of a cell to respond to an external stimulus is essential for the growth and survival of the cell and the organism. Typically, external factors that are designed to affect the cell bind to a receptor, which in turn triggers a signaling cascade that ultimately affects gene transcription. External stimuli can bind to receptors outside or inside of the cell. External stimuli can include growth factors, small peptides, cytokines, chemokines, ions, neurotransmitters, neurotrophins, extra-cellular matrix components, and hormones, as well as environmental stimuli and by-products of cellular metabolism.

Steroid hormones are critical for the development of all multicellular organisms. In plants, brassinosteroids (BRs) play a major role in promoting plant growth. Defects in steroid synthesis. such as BR synthesis, or steroid signaling cause multiple growth defects in both plants and animals, including dwarfism, sterility, abnormal vascular development, and photomorphogenesis in the dark. Brassinosteroids are a group of naturally occurring steroidal plant hormones that are required for plant growth and development. The first identified BR, Brassinolide, was discovered in 1973, when it was shown that pollen extract from Brassica napus could promote stem elongation and cell division. Physiological research indicates that exogenous brassinosteroids alone, or in combination with auxin, enhance bending of the lamina joint in rice. The total yield of Brassinosteroids from 230 kg of Brassica napus pollen, however, was only 10 mg. Extract from the plant Lychnis viscaria contains a relatively high amount of BRs. Lychnis viscaria is said to increase the disease resistance of surrounding plants. In Germany, extract from the plant is allowed for use as a “plant strengthening substance.” Since their initial discovery, over seventy BR compounds have been isolated from plants.

BRs have been shown to be involved in numerous plant processes: promotion of cell expansion and cell elongation; cell division and cell wall regeneration; promotion of vascular differentiation; pollen elongation for pollen tube formation; acceleration of senescence in dying tissue cultured cells; and providing protection during chilling and drought stress.

Treatment with low or high concentrations of brassinosteroids promotes or inhibits the growth of roots in rice, respectively (Radi et al. J. Crop Sci. 57, 191 198 (1988)). Brassinosteroids also promote the germination of rice seeds (Yamaguchi et al. Stimulation of germination in aged rice seeds by pre-treatment with brassinolide, in Proceeding of the fourteenth annual plant growth regulator society of America Meeting Honolulu, ed. Cooke A R), pp. 26 27 (1987)). The lamina joint of rice has been used for a sensitive bioassay of brassinosteroids (Maeda Physiol. Plant. 18, 813 827 (1965); Wada et al. Plant and Cell Physiol. 22, 323 325 (1981); Takeno et al. Plant Cell Physiol. 23, 1275 1281 (1982)), because of high sensitivity thereof to brassinosteroids. In etiolated wheat seedlings treatment with brassinolide or its derivative, castasterone, stimulates unrolling of the leaf blades (Wada et al. Agric. Biol. Chem. 49, 2249 2251 (1985)).

Brassinosteroids are recognized as a class of plant hormones through the combination of molecular genetics and researches on biosyntheses (Yokota Trends in Plant Sci., 2, 137 143 (1997)). Most of the C28-brassinosteroids are common vegetable sterols, and they are considered to be biosynthesized from campesterol, which has the same carbon side chain as that of brassinolide. The basic structure of BR is presented below.

Although the sites for BR synthesis in plants have not, to date, been experimentally demonstrated, one well-supported hypothesis is that as BR biosynthetic and signal transduction genes are expressed in a wide range of plant organs, all tissues produce BRs. Since the chemistry of brassinosteroids was established, biological activities of these homologues have been extensively studied, and their notable actions on plant growth have been revealed, which include elongation of stalks, growth of pollen tubes, inclination of leaves, opening of leaves, suppression of roots, activation of proton pump (Mananda, Annu. Rev. Plant Physiol. Plant Mol. Biol. 39, 23 52 (1988)), acceleration of ethylene production (Sch)agnhaufer et al., Physiol. Plant 61, 555 558 (1984)), differentiation of vessel elements (Iwasaki et al., Plant Cell Physiol., 32, pp. 1007 1014 (1991); Yamamoto et al. Plant Cell Physiol., 38, 980 983 (1997)), and cell extension (Azpiroz et al. Plant Cell, 10, 219 230 (1998)). Furthermore, mechanisms and regulations of physiological actions of brassinosteroids have been revealed by a variety of studies on their biosynthesis (Clouse, Plant J. 10, 1 8 (1996); Fujioka et al. Physiol. Plant 100, 710 715 (1997)).

### SUMMARY OF THE INVENTION

The present invention provides a novel method for regulating the signal transduction pathways in plants and animals. The present invention identifies a novel method for regulating the kinase activity affected by growth factors, such as brassinosteroids and insulin. The present invention provides a method of dephosphorylating kinase proteins, such as BIN2, GSK3. and homologs thereof. The present invention provides for dephosphorylating proteins through the use of a PP1 phosphatase protein. such as PP1 or BSU1.

The present inventions also provides methods for regulating GSK3 pathways in eukaryotic cell systems, such as in animals like mammals through the use of the BSU1 or PP1 phosphatases. The present invention provides for regulation of GSK3 and GSK3-related kinases through the use of PP1 phosphatase, such as PP1 and BSU1.

The present invention provides methods for modulating the growth or sterility/fertility of a cell comprising introducing into a cell a nucleic acid encoding a phosphatase that removes a phospho group from a tyrosine residue in GSK3 or BIN2 or functional equivalents or homologs thereof. The tyrosine residue to be dephosphorylated may correspond to tyrosine 279 of GSK3α, tyrosine 216 of GSK3β, or tyrosine 200 of BIN2.

The present invention also provides methods for screening a molecule for the ability to interact with a PP1 phosphatase polypeptide, such as PP1 or BSU1 polypeptides, comprising contacting a candidate molecule with a polypeptide that comprises (i) the amino acid sequence of BSU1 or PP1; or (ii) BSU1 or PP1 encoded by a polynucleotide comprising a nucleotide sequence at least 90% identical to BSU1 or to mammalian PP1, wherein the polypeptide is capable of dephosphorylating phosphorylated BIN2, under conditions and for a time sufficient to permit the candidate molecule and polypeptide to interact; and then detecting the presence or absence of binding of the candidate molecule to the polypeptide, and thereby determining whether the candidate molecule interacts with the BSU1 polypeptide.

The present invention further provides methods for treating diseases and/or conditions related to BIN2 or GSK3 activity comprising contacting a cell of the plant or animal with BSU1 or PP1 or functional equivalents or homolgs thereof or an agent that modulates the activity of BSU1 or PP1, wherein increasing the phosphatase activity in the cell by either increasing BSU1 or PP1 or functional equivalents or homolgs thereof phosphatase expression and/or enzymatic activity increases dephosphorylation of GSK3 or BIN2.

The present invention provides methods for identifying an agent that modulates brassinosteroid signaling comprising contacting a cell expressing a brassinosteroid receptor, BSU1 and BIN2 or GSK3 with a test agent, then contacting the cell with a brassinosteroid: and then detecting phosphatase activity of BSU1 on BIN2 or GSK3, wherein the presence of phosphatase activity indicates that the test agent modulates brassinosteroid activity.

The present invention also provides methods for identifying agents that modulate GSK3 activity comprising contacting a cell comprising GSK3 or a homolog thereof and BSU1 or a homolog thereof with a test agent, then contacting the cell with an agent known to activate GSK3 or the homolog thereof; and then detecting phosphatase activity of BSU1 or the homolog thereof on GSK3 or the homolog thereof. w herein the presence of phosphatase activity indicates that the agent modulates GSK3 activity.

### BRIEF DESCRIPTION OF THE DRAWINGS

FIGS. 1A-1E show that BR induces dephosphorylation of BIN2 and that BSU1 inhibits BIN2 phosphorylation of BZR1. FIG. 1A shows that BR induces dephosphorylation of BIN2. Total proteins of TAP-BIN2 transgenic plants treated with 0.25 μM brassinolide (BL) or mock solution for 2 hrs were analyzed by two-dimensional gel electrophoresis followed by immunoblotting using the peroxidase anti-peroxidase (PAP) antibody that detects TAP-BIN2. FIG. 1B shows that BSU1 does not dephosphorylate phospho-BZR1 in vitro. MBP-BZR1 was incubated with GST-BIN2 to produce phosphorylated BZR1 (pBZR1), and then GST-B1N2 was removed by glutathione-agarose. pBZR1 was then incubated with GST, GST-BSU1 or GST-BSL1 for 12 hrs and analyzed by immunoblotting using anti-MBP antibody. FIG. 1C shows that Pre-incubation of BSU1 and BIN2 reduces BZR1 phosphorylation. GST-BIN2 was pre-incubated with GST-BSU1 or GST for 0, 0.5, 1, 1.5 and 2 hrs before MBP-BZR1 and ³²P-γATP were added. FIG. 1D shows that BSU1inhibits BIN2 kinase activity for BZR1. Partially phosphorylated ³²P-MBP-pBZR1, prepared by incubation with GST-BIN2 and ³²P-γATP followed by affinity purification, was further incubated with GST-BIN2, GST-BSU1, or both, in the presence of non-radioactive ATP, and analyzed by autoradiography. GST-BIN2 M115A is a kinase-inactive mutant BIN2. FIG. 1E shows that BSU1 inhibits BIN2 but not bin2-1. ³⁵S·BSU 1-YFP plants were treated with 0.25 μM BL or mock solution for 30 min prior to protein extraction and immunoprecipitation. GST-BIN2 or GST-bin2-1 was first incubated with BSU1-YFP immunoprecipitated from BR-treated (+BL) or untreated plants, followed by removal of BSU1-YFP Protein A beads, and then incubated with MBP-BZR1 and ³²P-γATP. Col-0, immunoprecipitation from non-transgenic plant as control. CBB indicates Coomassie brilliant blue-stained gels.

FIGS. 2A-2D show that BSU1 directly interacts with BIN2 in vitro and in vivo. FIG. 2A shows that BSU1 interacts with BIN2 and bin2-1 in vitro. GST, GST-BIN2 and GST-bin2-1 were separated by SDS-PAGE and blotted onto nitrocellulose membrane. The blot was probed sequentially with MBP-BSU1 and anti-MBP antibody (upper) and then stained with Ponceau S (lower). FIG. 2B shows co-immunoprecipitation of BSU1 or BSL1 and BIN2. The protein extracts of the tobacco leaves transiently transformed with the indicated constructs were immunoprecipitated with anti-GFP antibody, and the immunoblot was probed with anti-myc and anti-GFP antibody. FIG. 2C shows that BiFC assay shows in vivo interaction between BSU1 or BSL1 and BIN2. The indicated constructs were transformed into tobacco leaf epidermal cells. Bright spots in BIN2-nYFP+cYFP are chloroplast auto-fluorescence. FIG. 2D shows that BR-induced interaction of BSU1 and BIN2. Arabidopsis plants (F1) expressing BSU1-YFP or co-expressing BSU1-YFP and BIN2-myc were grown on the medium containing BR biosynthetic inhibitor, brassinazole (BRZ), for 10 days. The plants were treated with 10 μM MG-132 for 1 hr and then with 0.2 μM BL or mock solution for 15 min. Total protein extracts were immunoprecipitated with anti-myc antibodies, and the immunoblot was probed with anti-GFP and anti-myc antibodies.

FIGS. 3A-3G show that BSU1 regulates BIN2 but not bin2-1 in vivo. FIG. 3A shows subcellular localization of BZR1-YFP in the cells co-transformed with the indicated constructs. FIG. 3B shows immunoblots of BZR1-YFP proteins obtained from the tobacco leaves co-transformed with constructs indicated. The upper band is phosphorylated BZR1 and lower one unphosphorylated. FIG. 3C shows overexpression of BSU1-YFP reduces the accumulation of BIN2-myc protein in a transgenic Arabidopsis line. Heterozygous ³⁵S-BIN2-myc and ³⁵S::BIN2-myc/³⁵S-BSU1-YFP plants (F1) were treated with 0.25 .tM BL or mock solution for 30 min. Immunoblot was probed with anti-myc or anti-GFP antibodies, and a non-specific band serves as loading control. FIG. 3D shows BSU1 reduces the accumulation of BIN2 but not that of bin2-1 . BIN2- or bin2-1-myc levels were analyzed by anti-myc antibody in tobacco cells co-expressing myc-tagged BSU1 or BSU1-D51ON mutant protein. A nonspecific band serves as loading control. FIG. 3E shows overexpression of BSU1-YFP (1-BSU1) partially rescues the bril-116 mutant, but not the bin2-1 mutant. FIG. 3F shows hypocotyl phenotypes of seedlings (genotype shown) grown in the dark on MS medium for 5 days. Bottom two panels show confocal images of BSU1-YFP in the plants indicated. FIG. 3G shows quantitative RT PCR analysis of SAUR-AC1 RNA expression in wild type (bril-116 (+/−)), bril-116 (−/), and BSU1-YFP/bril-116 plants. Error bars indicate standard error.

FIG. 4 BSU1 dephosphorylates the pTyr200 residue of BIN2 but not that of bin2-1 mutant. (a) Tyr200 phosphorylation of BIN2 is required for its kinase activity. GST BIN2 or GST-BIN2 Y200F was incubated with MBP-BZR1 and ³²P-γATP. CBB indicates Coomassie brilliant blue-staining. (b-c) BSU1 dephosphorylates pTyr200 of BIN2 but not that of bin2-1 in vitro. (b) Gel blots of GST-BIN2, GST-BIN2 Y200A and GST-bin2-I mutant proteins incubated with MBP or MBP-BSU1 were probed with the anti-pTyr antibody and then with anti-GST antibody. (c) GST-BIN2 and GST-bin2-1 were incubated with BSU1-YFP immunoprecipitated from transgenic Arabidopsis. (Y) indicates relative signal level of pTyr200 normalized to total GST-B1N2 or GST-bin2-1 protein. (d-e) pTyr200 residue of endogenous BIN2. but not mutant bin2-1, is dephosphorylated by BR treatment. (d) The det2 mutant was treated with 10 μM MG132 for 1 hr prior to treatment with 0.2 μM BL for the indicated time. BIN2 protein was immunoprecipitated with a polyclonal anti-serum for BIN2. Gel blot was probed with anti-pTyr. anti-BIN2 serum, and anti-GSK3 a/P antibody. (e) Transgenic plants expressing BIN2-myc or bin2-1-myc was pretreated with 10 μM MG132 and then treated with 0.25 μM BL (+BL) or mock solution (−BL). BIN2-myc and bin2-1-myc were immunoprecipitated by anti-myc antibody and gel blots were probed with antibodies indicated. pTyr200 was detected with monoclonal anti-phospho-Tyr279/216 GSK3α/β (anti-pTyr) antibody. (f, g) Phosphorylation of Tyr200 is required for BIN2 inhibition of plant growth. (f) Overexpression of BIN2-YFP but not BIN2-Y200E-YFP causes severe dwarf phenotypes in TI generation. Upper left panel shows zoom-in view. Lower panel shows BIN2-YFP and BIN2-Y200F protein levels detected by anti-YFP antibodies. A nonspecific band serves as loading control. (g) Dwarf phenotypes were caused by overexpressing bin2-1-myc but not by bin2-1-Y200E-myc. Seventy -six of a total 281 ³⁵S.:bin2-/-myc transgenic T1 seedlings showed dwarfism while none of a total 412 ³⁵S.:bin2-/-Y200E-myc transgenic T1 plants showed dwarf phenotype. (h-j) Loss of function of four BSU1 family members causes extreme dwarfism and reduced BR-responsive gene expression in Arabidopsis. An artificial microRNA construct for suppressing BSL2 and BSL3 (BSL2,3-amiRNA) was introduced into bsul bs11 double knockout mutant. (h) Eight of 27 T1 transgenic plants showed dwarf phenotypes similar to those of strong BR-deficient mutants, with short petiole and round-shape leaves. Right panel shows zoom-in view of the quadruple mutant. (i) Hypocotyl phenotypes of 5-day old dark-grown seedlings of bsulbs11/BSL2,3-amiRNA compared with Col-O, bin2-1 (−/−) and bril-116. 0) Quantitative RT-PCR analysis of SAUR-AC1 RNA expression in bsulbs11/BSL2,3-amiRNA and Col-0 plants. Bars indicate standard error.

FIGS. 5A-5G show regulation of the BIN2 homolog, AtSK12 BSU1-mediated tyrosine dephosphorylation. FIG. 5A shows phylogenetic tree of the ten Arabidopsis GSK3/Shaggy-like kinases (AtSKs). FIG. 5B shows six AtSKs specifically interact with BZR1 in yeast two-hybrid assays. Activation domain (AD) fused AtSKs were transformed into the cells containing DNA binding domain (BD) fused BZR1. Yeast clones were grown on Synthetic Dropout (SD) or SD-Histidine medium. FIG. 5C shows both AtSK12 and BIN2 interact with BZR1 in BiFC assays. Transgenic Arabidopsis plants expressing nYFP-BIN2, nYFP-AtSK12 and nYFP-AtSK12-cd (C-terminal 29 amino acid deletion) were crossed to BZR1-cYFP plants, respectively. The seedlings of F1 generation were grown in white light for 7 days and YFP signals of epidermal cells were observed. FIG. 5D shows various phenotypes of transgenic plants (T1) overexpressing WT AtSK12 or AtSK12-E297K. FIG. 5E shows AtSK12 phosphorylates BZR1 in vitro. GST-AtSK12 was incubated with MBP-BZR1 and ³²P-γATP. CBB indicates Coomassie brilliant blue-stained gel. FIG. 5F shows BR induces degradation of AtSK12. Homozygous plants expressing AtSK12-myc were treated with 0.25 MM BL for 30 min. Proteins immunoprecipitated by anti-myc antibodies were blotted onto nitrocellulose membrane and probed by anti-myc antibody. FIG. 5G shows overexpression of BSU1-YFP reduces the accumulation of AtSK12-myc protein in a transgenic Arabidopsis plant. (h) BR induces pTyr dephosphorylation of AtSK12. Homozygous AtSK12-myc plants were pretreated with 10 μM MG132 and then treated with 0.25 μM BL (+BL) or mock solution (−BL). AtSK12-myc was immunoprecipitated by anti-myc antibody and gel blots were probed with anti-pTyr and anti-myc antibodies.

FIGS. 6A-6E show BSK1 directly interacts with BSU1. FIG. 6A shows BSK1 binds to BSU1 in vitro. The GST fusion proteins of the kinase domains of BR11 (GST BR11-K) and BAK1 (GST-BAK1-K) and full-length BSK1 (GST-BSK1) were separated by SDS-PAGE and blotted onto nitrocellulose membrane. The blot was probed sequentially with MBP-BSU1 and anti-MBP antibody (upper) and then stained with Ponceau S (lower). FIG. 6B shows BiFC assays show in vivo interaction between BSU1 or BSL1 and BSK1. Tobacco leaf epidermal cells were transformed with indicated constructs. At5g49760 is a receptor kinase unrelated to BR signaling used here as a negative control. Bright spots in nYFP+BSK 1-cYFP and At5g49760-nYFP+BSK1-cYFP are chloroplast auto-fluorescence. FIG. 6C shows co-immunoprecipitation of BSK1 and BSU1. Total protein extracts obtained from Arabidopsis plants (F1) expressing BSU1 -YFP or co-expressing BSU1-YFP and BSK1-myc were immunoprecipitated with anti-myc, and the immunoblot was probed with anti-GFP and anti-myc antibody. FIG. 6D shows BSK1 phosphorylation BRI1 enhances BSK1 binding to BSU1. GST-BSK1 or GST-BSK1 S230A was incubated with GST-BRI1-K or GST for 2 hrs. Overlay assay was performed as described in A. FIG. 6E shows the BR signal transduction pathway. Components in active states are in red color and inactive states in blue. In the absence of BR (−BR), BRI1 is kept in an inactive form with help of its inhibitor BKII, and consequently BAKI, BSK1 and BSU1 are inactive, while BIN2 is active and phosphorylates BZR1 and BZR2 (BZR1/2), leading to their degradation, loss of DNA binding activity, and exclusion from the nucleus by the 14-3-3 proteins. In the presence of BR (+BR), BR binding to the extracellular domain of BRI1 induces dissociation of BKI1 and association and inter-activation between BRI1 and BAKI. Activated BRI1 then phosphorylates BSK1, which in turn dissociates from the receptor complex and interacts with and presumably activate BSU1. BSU1 inactivates BIN2 by dephosphorylating its pTyr200, allowing accumulation of unphosphorylated BZR1/2, likely with help of a phosphatase that is yet to be identified. Unphosphorylated BZR1/2 accumulate in the nucleus and alter the expression of BR-target genes, leading to cellular and developmental responses. While individual representative protein is shown for each function, in Arabidopsis most of these components have about 2 to 5 homologous proteins (paralogs) that can contribute to the same or similar signaling function.

FIGS. 7A-7B show the model of the BR signal transduction pathway before (FIG. 7A) and after (FIG. 7B) this study. In the absence of BR, the GSK3-like kinase BIN2 phosphorylates two transcription factors, BZR1 and BZR2 (pBZR1/2), to inhibit BR-responsive gene expression. Upon activation by BR binding, BRI1 receptor kinase phosphorylates BSKs, and this leads to accumulation of dephosphorylated BZR1 and BZR2, most likely by inhibiting BIN2 or activating BSU1. FIG. 7A shows in previous models of BR signaling, BSU1 was proposed to mediate dephosphorylation of BZR1 and BZR2, and the mechanism for inhibiting BIN2 kinase remains unknown. FIG. 7B shows results of this study demonstrate that BSU1 does not directly dephosphorylate BZR1 or BZR2. Instead, it dephosphorylates BIN2 at tyrosine 200 to inactivate BIN2 kinase activity and inhibit BIN2 phosphorylation of BZR1 and BZR2. BR-activated BRI1 phosphorylates BSKs to promote its binding and activation of BSU1. Arrows show promotion actions and bar ends show inhibitory actions. Solid lines show direct regulation, and dotted lines indicate hypothetical regulation.

FIG. 8 shows overexpression of BSL1 suppresses the phenotype of the bril-5 mutant. The bril-5 overexpressing BSL1-YFP (BSL1-YFP/bril-5, left) and untransformed bril-5 (right) were grown in soil for six weeks.

FIGS. 9A-9B show BSU1 and BSL1 purified from E.coli are manganese-dependent phosphatases. FIG. 9A shows both GST-BSU1 and its homolog, GST-BSL1 dephosphorylate phospho-myelin basic protein. FIG. 9B shows GST-BSU1 requires manganese ion for its activity. All metal ions were added to the phosphatase reactions as 1 mM final concentration.

FIGS. 10A-10B show BSU1 and BSL1 inhibit B1N2 phosphorylation of BZR1 and BZR2. GST-BIN2 and GST-BSU1 or GST-BSL1 were co-incubated with MBP-BZR1 (FIG. 10A) or MBP-BZR2 (FIG. 10B) and 32P-γATP for 3 hrs at 30° C. CBB indicates Coomassie brilliant blue stained-gel. FIGS. 10C-10D show that BSU1 and BSL1 do not dephosphorylate phosphorylated BZR1 and BZR2 in vitro. 32P-pBZR1 and 32P-pBZR2 were prepared by incubation with GST-BIN2 and ³²P-γATP followed by removal of GST-BIN2 and ³²P-γATP by sequential purification using glutathione and amylose beads. Pre-labeled ³²P-pBZR1 (FIG. 10A) and ³²P-pBZR2 (FIG. 10B) were then incubated with GST, GST-BSU1 and GST-BSL1, respectively, for 16 hrs at 30° C. CBB indicates Coomassie brilliant blue stained-gel.

FIG. 11 shows BSU1 and BSU1 phosphatase domain inhibit BIN2 phosphorylation of BZR1. GST-BIN2 and GST-BSU1 or GST-BSU1-P (C-terminal phosphatase domain) or GST-BSU1 -KL (N- terminal Kelch domain) were pre-incubated for 1 hr, and then incubated with MBP-BZR1 and ³²P-γATP for 3 hrs at 30° C. CBB indicates Coomassie brilliant blue stained-gel.

FIGS. 12A-12B show BSU1-YFP inhibits BIN2 activity but does not dephosphorylate phosphorylated BZR1. BSU1-YFP protein was immunoprecipitated (IP) from 35S-BSU1-YFP transgenic Arabidopsis plants. FIG. 12A shows BSU1-YFP was incubated for 3 hrs with pre-phosphorylated 32P-MBP-BZR1 after removal of GST-BIN2. FIG. 12B shows BSU1-YFP immunoprecipitated from plants treated with 0.5 RM BL or mock solution was incubated with GST-BIN2, MBP-BZR1 and ³²P-γATP for 3 hrs. CBB indicates Coomassie brilliant blue stained-gel.

FIG. 13 shows BSU1 interacts with BIN2 and bin2-1. BIN2-myc and bin2-myc proteins expressed in transgenic Arabidopsis were immunoprecipitated (IP) by anti-myc antibody, and the beads were then incubated with extracts of BSU1-YFP overexpressing plants. Immunoblot was probed with anti-myc and anti-GFP antibody. Col-0, wild type plants expressing no BIN2-myc.

FIG. 14 shows in vivo interactions between BSU1 or BSL1 and BIN2 or bin2-1 in BiFC assays. Cells co-transformed with BIN2 or bin2-1 fused N-terminal half (nYFP) and BSU1 or BSL1 fused C-terminal half (cYFP) of yellow fluorescence protein (YFP) showed good fluorescence signal consistent with their subcellular localization patterns, whereas cells co-expressing BIN2 or bin2-1-nYFP and non-fusion cYFP showed only auto-fluorescence of chloroplast.

FIGS. 15A-15B show distinct subcellular localization patterns of BSU1 and BSL1 in transgenic Arabidopsis plants. Confocal images show BSU1-YFP (FIG. 15A) and BSL1-YFP (FIG. 15B) signal in hypocotyls of Arabidopsis seedlings grown in the dark for 5 days.

FIGS. 16A-16B show the substitution of BSU1 Asp510 to Asn abolishes its phosphatase activity. FIG. 16A shows phosphatase assay using phospho-myelin basic proteins as a substrate showed that BSU1-D5ION mutant has about 15% phosphatase activity of the wild type protein. GST and GST-Kelch domain of BSU1 were used as negative control. FIG. 16B shows BSU1-D51ON-YFP (left) shows same subcellular localization pattern as wild type BSU1-YFP (right) in Arabidopsis leaf epidermal cells.

FIGS. 17A-17B show BSU1-D51ON overexpression cannot decrease the BIN2-myc protein amount in Arabidopsis. FIG. 17A shows immunoblot of total proteins was probed with anti-myc and anti-GFP antibody. FIG. 17B shows BIN2-myc mRNA level in BSU-YFPxBIN2-myc is similar to Col-OxBIN2-myc. Semi-quantitative RT-PCR analysis was performed to compare BIN2-myc mRNA expression level. PP2A (At1g13320) was used as normalization control.

FIG. 18 shows BR treatment reduces the level of BIN2 but not bin2-1 proteins. Tobacco leaves transformed with 35S-BIN2-myc or 35S-bin2-1-myc constructs were treated with 1 μM BL for 1 hr. Immunoblot of total proteins was probed with anti-myc antibody.

FIGS. 19A-19C show the bin2-1 mutation suppresses the BSU1-overexpression phenotypes. (FIG. 19A) Homozygous bin2-1 (left) and bin2-1/bsul-D (right) plants. (FIGS. 19B-19C) Genotyping of plants shown in FIG. 3e. FIG. 19B shows the DNA fragments containing bin2-1 mutation site amplified by PCR were digested with Xho1 restriction enzyme. FIG. 19C shows BSU1-YFP DNA fragments were amplified with PCR using 35S promoter- and BSU1-specific primers.

FIG. 20 shows mass spectrometry analysis of BIN2 auto-phosphorylation site. GST-BIN2 protein purified from E.coli was subjected to in vitro kinase reaction. The protein was digested by trypsin and analyzed by LC-MS/MS using LTQ/FT mass spectrometry. The CID mass spectrum and sequence of the peptide containing phospho-tyrosine 200 residue of BIN2 are shown.

FIG. 21 shows amino acids alignment of the immunogen peptide of phospho-tyrosine 279/216 GSK3 a/p antibody and the same region of BIN2. The phospho-tyrosine residue is marked by asterisk.

FIG. 22 shows anti-phospho-Tyr279/216 GSK3α/β antibody specifically detects phospho-tyrosine 200 of BIN2. Immunoblot of the wild type, the kinase inactive M115A, and the Y200A mutant GST-BIN2 proteins were probed with the anti-phospho-Tyr279/216 GSK3α/β antibody. The blot was re-probed with anti-GST antibody.

FIG. 23 shows BR induces degradation of BIN2 but not bin2-1. Transgenic plants expressing BIN2-myc or bin2-1-myc were treated with 0.25 μM BL for 30 min. Proteins immunoprecipitated by anti-myc agarose were blotted onto nitrocellulose membrane and probed by anti-myc antibody.

FIG. 24 shows both AtSK12 and BIN2 interact with BZR1 in BiFC assay. Transgenic Arabidopsis plants expressing nYFP-BIN2, nYFP-AtSK12 and nYFP-AtSK12-cd (C-terminal 29 amino acid deletion) were crossed into BZR1-cYFP plants, respectively. The seedlings of F1 generation were grown in the dark for 4 days and YFP signals of hypocotyls were observed.

FIGS. 25A-25B show effect of brassinazole (BRZ) and brassinolide (BL) on localization and accumulation of AtSK12. FIG. 25A shows confocal images of hypocotyls cells of transgenic Arabidopsis plants expressing YFP-AtSK1 2 grown on MS medium, or MS containing 2 pM BRZ or 0.1 pM BL in the dark for 4 days. FIG. 25B shows BRZ induces the accumulation of AtSK1 2. AtSK1 2-myc plants were grown on MS or 2 pM BRZ medium for 5 days. Total protein extracts were blotted onto nitrocellulose membrane and probed by anti-myc antibody.

FIG. 26 shows mass spectrometry analysis of AtSK12 autophosphorylation site. GST-AtSK12 protein purified from E.coli was subjected to in vitro kinase reaction. The protein was digested by trypsin and analyzed by LC-MS/MS using LTQ/FT mass spectrometry. The CID mass spectrum and sequence of the peptide containing phospho-tyrosine 233 residue of AtSK12 are shown.

FIG. 27 shows comparison of tissue specific gene expression between BSU1 and BRI1, BSK1, BIN2, or BZR1. As indicated by the small graph in left bottom of each image, the higher level of expression for BSU1 is shown in red and higher expression of its counterpart is shown in blue. Yellow color indicates similar expression level. Figures were obtained from online Arabidopsis eFP browser (http:libbc.botanvutororite.caefpfc(ji_biniefDWeb.cgi) (Winter et al., 2007. PLoS One 2(8): 2718).

FIG. 28 shows BSU1 shows tyrosine phosphatase activity. MBP, MBP-Ketch (N-terminal domain of BSU1), or MBP-BSU1 was incubated with p-nitrophenyl phosphate as a substrate. The enzyme activity was determined by production of p-nitrophenol.

FIG. 29 shows that PP1 dephosphorylates BIN2. A GST-tagged BIN2 was isolated from cells and incubated with PP1 purified from E. coli cells expressing the phosphatase. The presence of PP1 increased dephosphorylation of BIN2 tyrosine200. The PP1 inhibitior, PP2 (protein phosphatase inhibitor 2), inhibited the enzymatic activity of the PP1 phosphatase on BIM. Similarly, the phosphatase inhibitor, manganese chloride also inhibited the enxymatic activity of PP1 on BIN2.

FIG. 30 shows that human protein phosphatase 1 gamma (PP1γ) dephosphorylates tyrosine 216 of human GSK3 beta in vitro. MBP or MBP-fused protein phosphatase 1 gamma (MBP-hsOPP1cc) was incubated with GST-fused human GSK3 beta protein. The proteins were resolved by SDS-PAGE and transferred to a membrane for immunoblotting. Tyrosine 216 phosphorylation status of GSK3 beta was detected using anti-phospho-tyrosine 216 antibody. The lower panel is a Ponceau stain of the membrane.

### DETAILED DESCRIPTION

In the 1990s. it was discovered in Arabidopsis that BRs are essential plant hormones through analysis of mutant plants unable to naturally synthesize BRs. These Arabidopsis mutants which show characteristic dwarfism, e.g., dwfl: Feldman et al. Science 243, 1351 1354 (1989); dim: Takahashi et al. Genes Dev. 9, 97 107 (1995); and cbb1: Kauschmann et al. Plant J. 9, 701 703 (1996) and their corresponding structural photomorphogenesis and dwarfism are known (e.g. cpd: Szekeres et al. Cell, 85, 171 182 (1997)) and de-etiolation (det2: Li et al., Science 272, 398 40) (1996); Fujioka et al. Plant Cell 9, 1951 1962 (1997)). The morphologic changes are directly related to their deficiency in BR biosynthesis. BRs are also essential in other plants, as demonstrated with studies on a dwarf mutant of Pisum sativum (Nomura et al. Plant Physiol. 113, 31 37, 1997). In all these mutant plants, use of brassinolide will negate the severe dwarfism.

The mechanism by which BR can propagate its effects starts with a cell receptor to interact with a BR. Unlike animal steroid hormones, which act through nuclear receptors, BRs bind to a receptor kinase (BRI)) at the cell surface to activate the BR response transcription factors named BZR1 and BZR2 (also known as BES1) through a signal transduction pathway. Receptors may be located on the surface of a cell, or within the cell itself. Cell-surface receptor kinases activate cellular signal transduction pathways upon perception of extracellular signals, thereby mediating cellular responses to the environment and to other cells. The Arabidopsis genome encodes over 400 receptor-like kinases (RLKs) (Shiu et al., Plant Cell 16, 1220 (May, 2004)). Some of these RLKs function in growth regulation and plant responses to hormonal and environmental signals. However. the molecular mechanism of RLK signaling to immediate downstream components remains poorly understood, as no RLK substrate that mediates signal transduction has been established in Arabidopsis (Johnson et al., Curr Opin Plant Biol 8, 648 (December, 2005)).

The use of Brassinosteroid-insensitive Arabidopsis mutants allowed for the identification of several components of Brassinosteroid signal transduction, including the leucine-rich-repeat (LRR) receptor-like kinases (RLK), brassinosteroid-insensitive 1 (BRI1) and BRI1-associated receptor-kinase (BAK1), the glycogen synthase kinase 3 (GSK3)-like kinase brassinosteroid-insensitive 2 (BIN2), the phosphatase bril suppressor 1 (BSU1), and two transcription factors brassinazole-resistant 1 (BZR1) and brassinazole resist/n12 (BZR2)/bri/-EMS-suppressor 1 (BES1). Meanwhile, it has been reported that genetic regulation of the brassinosteroid metabolism makes plants highly sensitive to brassinosteroids, and thus an effect of brassinosteroid administration is markedly enhanced (Neff et al. Proc. Natl. Acad. Sci., USA 96, 15316 23 (1999)).

The upstream BR-signaling components at the plasma membrane include BRI1 and BAK1 receptor kinases, a novel protein (BKI 1) that inhibits BRI1, and the plasma membrane associated BR-signaling kinases (BSKs). BR binding to the extracellular domain of BRI1 causes disassociation of BKI1 from BRI1 and induces association and trans-phosphorylation between BR11 and its co-receptor BAK1, leading to activation of BRI1 kinase and phosphorylation of its substrates BSKs. Genetic studies supported an essential role for BSKs in transducing the signal to the downstream components, but their direct target remains unknown.

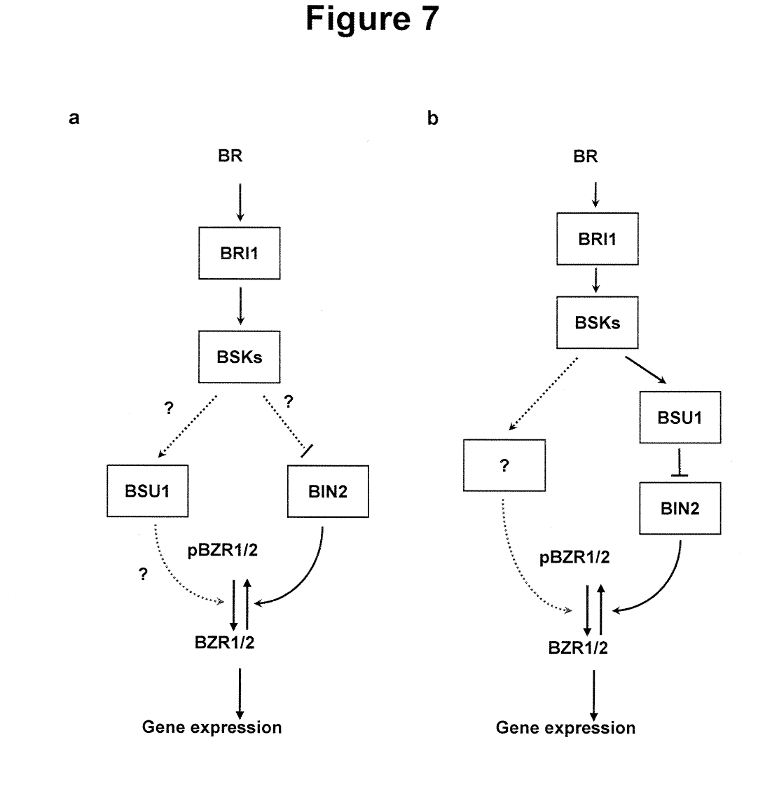

Downstream BR signaling involves the GSK3-like kinase BIN2, the Kelch-repeats-containing phosphatase BSU1, the 14-3-3 family of phosphopeptide-binding proteins, and BZR1 and BZR2, which directly bind DNA and regulate BR-responsive gene expression. As a negative regulator of BR signaling, BIN2 phosphorylates BZR1 and BZR2 at numerous sites to inhibit their activities through multiple mechanisms. These include accelerating proteasome-mediated degradation, promoting nuclear export and cytoplasmic retention by the 14-3-3 proteins, and inhibiting DNA binding and transcriptional activity. By contrast, the BSU1 phosphatase is a positive regulator of BR signaling. Overexpression of BSU1 increases the dephosphorylated BZR2/BES1 and activates BR responses. However, BSU1 does not interact with or effectively dephosphorylate BZR2/BES1 in vitro and the biochemical function of BSU1 remains unknown. It is believed that BR induces rapid dephosphorylation of BZR1 and BZR2 by inhibiting BIN2 and/or activating BSU1 . However, the mechanisms by which upstream BR signaling regulates BIN2 and BSU1 remain unclear (FIG. 7A). It has previously been understood in the art that brassinosteroids exert their signaling through BSK which in turn indirectly inhibit BIN2. However, the intermediate steps through activation of the BSK kinases and inhibition of the BIN2 signaling were unknown. Thus, a need was felt in the art to identify the mechanism by which brassinosteroid receptor activation leads to BIN2 inhibition.

Brassinosteroid. or BR, as used herein. refers to a plant growth regulator with a steroid backbone. It is known in the art that brassinosteroids have many functions, such as enhancement of plant growth and plant maturation, and induction of cold and heat resistance. Brassinolide is a type of brassinosteroid. Auxin is a plant growth regulator with an indole backbone that interacts with brassinosteroid signaling. It is known that some important roles of plant auxins include plant growth and differentiation, formation of flower buds and fruits, and responses to light and gravity.

Brassinosteroid (BR) regulates gene expression and plant development through a receptor kinase-mediated signal transduction pathway. Despite many components of the pathway identified, how the BR signal is transduced from the cell surface to the nucleus remains unclear. The present invention describes a complete BR signaling pathway by elucidating the key missing steps of the pathway. The present invention reveals that phosphorylation of BSK1 by the BR receptor kinase BRI1 promotes BSK1 binding to the BSU1 phosphatase, and BSU1 inactivates the GSK3-like kinase BIN2 by dephosphorylating a conserved phospho-tyrosine residue (pTyr200).

Mutations that affect phosphorylation/dephosphorylation of BIN2 pTyr200 (bin2-1, bin2-Y200F and quadruple loss-of-function of BSU1-related phosphatases) demonstrate an essential role for BSU1-mediated BIN2 dephosphorylation in BR-dependent plant growth. These results demonstrate direct sequential BR activation of BRI1, BSK1. and BSU1, and inactivation of BIN2, leading to accumulation of unphosphorylated BZR transcription factors in the nucleus. The present invention establishes a fully connected BR signaling pathway and provides an understanding of the mechanism of GSK3 regulation.

Steroid hormones are critical for development of all multicellular organisms. In plants, brassinosteroids (BRs) playa major role in promoting plant growth. Defects in BR synthesis or signaling cause multiple growth defects, including dwarfism, sterility. abnormal vascular development, and photomorphogenesis in the dark. Unlike animal steroid hormones, which act through nuclear receptors. BRs bind to a receptor kinase (BRI1) at the cell surface to activate the BR response transcription factors named BZR1 and BZR2 (also known as BES1) through a signal transduction pathway. Although many components have been identified and studied in detail, the understanding of the BR signaling pathway contained major gaps between the receptor kinases at the cell surface and downstream components in the cytoplasm and nucleus. (FIG. 7A).

The present invention closes the major gaps of the BR pathway by elucidating the biochemical function of the BSU1 phosphatase and the mechanism for regulating BIN2. The present invention shows that BR signaling inactivates BIN2 through BSU1-mediated dephosphorylation at a tyrosine residue that is conserved in all GSK3s and required for kinase activity. BSU1 directly interacts with BSK1 that has been phosphorylated by BRI1. The present invention provides key missing connections and establishes a complete signaling cascade from steroid binding at the cell surface to gene expression in the nucleus (FIG. 7B). The present invention also discloses a novel GSK3 regulation mechanism that appears to be ancient in evolution.

Phosphorylation of proteins is a fundamental mechanism for regulating diverse cellular processes. Protein phosphorylation occurs at tyrosine, serine and threonine residues. The protein phosphorylation and the regulation thereof are important in growth factor signal transduction, cell cycle progression and neoplastic transformation (Hunter et al., Ann. Rev. Biochem. 54:987-930 (1985), Ullrich et al., Cell 61:203-212 (1990), Nurse, Nature 344:503-508 (1990), Cantley et al, Cell 64:281-302 (1991)). The protein phosphatases are composed of at least two separate and distinct families (Hunter, T. (1989) supra) the protein serine/threonine phosphatases and the protein tyrosine phosphatases (PTPases).

The protein tyrosine phosphatases (PTPases) have been classified into two subgroups. The first subgroup is made up of the low molecular weight. intracellular enzymes that contain a single conserved catalytic phosphatase domain. All known intracellular type PTPases contain a single conserved catalytic phosphatase domain. Examples of the first group of PTPases include (1) placental PTPase 1B (Charbonneau et al.. Proc. Natl. Acad. Sci. USA 86:5252-5256 (1989); Chernoff et al., Proc. Natl. Acad. Sci. USA 87:2735-2789 (1989)), (2) T-cell PTPase (Cool et al., Proc. Natl. Acad. Sci. USA 86:5257-5261 (1989)), (3) rat brain PTPase (Guan et al.. Proc. Natl. Acad. Sci. USA 87:1501-1502 (1990)), (4) neuronal phosphatase (STEP) (Lombroso et al.. Proc. Natl. Acad. Sci. USA 88:7242-7246 (1991)), and (5) cytoplasmic phosphatases that contain a region of homolog) to cytoskeletal proteins (Gu et al., Proc. Natl. Acad. Sci. USA 88:5867-57871 (1991); Yang et al., Proc. Natl. Acad. Sci. USA 88:5949-5953 (1991)). Enzymes of this class are characterized by an active site motif of CX₅R. Within this motif the cysteine sulfur acts as a nucleophile which cleaves the P-O bond, releasing the phosphate. The arginine assists to interact with the phosphate and facilitate nucleophilic attack. The second subgroup of protein tyrosine phosphatases is made up of the high molecular weight, receptor-linked PTPases, termed R-PTPases. R-PTPases consist of an intracellular catalytic region, a single transmembrane segment, and a putative ligand-binding extracellular domain (Gebbink et al., supra). Dual-specificity phosphatases (dual-specificity protein tyrosine phosphatases) are phosphatases that dephosphorylate both phosphotyosine and phosphothreonine/serine residues (Walton et al., Ann. Rev. Biochem. 62:101-120, 1993).

The present invention provides a novel method for regulating the signal transduction pathways in plants and animals. The present invention identifies a novel method for regulating the kinase activity affected by growth factors, such as brassinosteroids and insulin. The present invention provides a method of dephosphorylating kinase proteins, such as BIN2, GSK3, and homologs thereof. The present invention provides for dephosphorylating proteins through the use of BSU1 and PP1 as a phosphatase protein.

The present inventions also provides methods for regulating GSK3 pathways in eukaryotic cell systems, such as in animals like mammals through the use of the BSU1 or PP1 phosphatases. The present invention provides for regulation of GSK3 and GSK3-related kinases through the use of PP1 and PP1 phosphatases. such as BSU1. PP1 phosphatases include PPP1 (such as hsPPPIce (SEQ ID NO: 31), hsPPP)cb (SEQ ID NO: 32), and hsPPP 1 ca (SEQ ID NO: 33)), BSU1 (SEQ ID NO: 27). BSL1 (SEQ ID NO: 28), BSL2 (SEQ ID NO: 29), and BSL3 (SEQ ID NO: 30).

An antibody refers to an immunoglobulin molecule or a fragment of an immunoglobulin molecule having the ability to specifically bind to a particular antigen. Antibodies are well known to those of ordinary skill in the science of immunology. As used herein, the term “antibody” refers to not only full-length antibody molecules but also fragments of antibody molecules retaining antigen binding ability. Such fragments are also well known in the art and are regularly employed both in vitro and in vivo. In particular, as used herein, the term “antibody” means not only full-length immunoglobulin molecules but also antigen binding active fragments such as the well-known active fragments F(ab′)2, Fab, Fv, and Fd.

As used herein, “subject” may include the recipient of the treatment to be practiced according to the invention. The subject may be a plant. The subject can be any animal. including a vertebrate, such as a mammal, for example a domestic livestock, laboratory subject or pet animal. The subject may be a human.

As used herein with respect to proteins and polypeptides, the term “recombinant” may include proteins and/or polypeptides and/or peptides that are produced or derived by genetic engineering, for example by translation in a cell of non-native nucleic acid or that are assembled by artificial means or mechanisms.

As used herein with respect to polypeptides and proteins, the term “isolated” may include a polypeptide or nucleic acid that, by the hand of man, exists apart from its native environment and is therefore not a product of nature. For example, an isolated polypeptide may exist in a purified form or may exist in anon-native environment such as, for example, a recombinant host cell.

The term “cDNA” refers to a DNA molecule which can be prepared by reverse transcription from a mature, spliced, mRNA molecule obtained from a cell, preferably a eukaryotic cell. cDNA lacks intron sequences that are usually present in the corresponding genomic DNA. The initial, primary RNA transcript is a precursor to mRNA which is processed through a series of steps before appearing as mature spliced mRNA. These steps include the removal of intron sequences by a process called splicing. cDNA derived from mRNA lacks. therefore, intron sequences.

As used herein, the term “analog” may include any polypeptide having an amino acid sequence substantially identical to a polypeptide. or peptide, of the invention, in which one or more residues have been conservatively substituted with a functionally similar residue. and further which displays substantially identical functional aspects of the polypeptides as described herein. Examples of conservative substitutions include substitution of one non-polar (hydrophobic) residue for another (e.g. isoleucine. valine, leucine or methionine) for another. substitution of one polar (hydrophilic) residue for another (e.g. between arginine and lysine, between glutamine and asparagine, between glycine and serine), substitution of one basic residue for another (e.g. lysine. arginine or histidine), or substitution of one acidic residue for another (e.g. aspartic acid or glutamic acid).

As used herein, a “homolog” may include any polypeptide having a tertiary structure substantially identical to a polypeptide of the invention which also displays the functional properties of the polypeptides as described herein. For example, a GSK3 homolog is a polypeptide possessing the same activities as GSK3α and/or GSK3β and/or BIN2.

As used herein, “pharmaceutically acceptable carrier” may include any material which, when combined with an active ingredient, allows the ingredient to retain biological activity and is non-reactive with the subject's immune system. Examples may include, but are not limited to, standard pharmaceutical carriers such as a phosphate buffered saline (PBS) solution, water, emulsions, and various types of wetting agents.

As used herein, “fusion” may refer to nucleic acids and polypeptides that comprise sequences that are not found naturally associated with each other in the order or context in which they are placed according to the present invention. A fusion nucleic acid or polypeptide does not necessarily comprise the natural sequence of the nucleic acid or polypeptide in its entirety. Fusion proteins have the two or more segments joined together through normal peptide bonds. Fusion nucleic acids have the two or more segments joined together through normal phosphodiester bonds.

A preparation of a polynucleotide encoding a kinase or fragment thereof and/or a phosphatase or fragment thereof may be a substantially pure polynucleotide that is free of other extraneous or unwanted nucleotides and in a form suitable for use within genetically engineered protein production systems. The term substantially pure polynucleotide is synonymous with the isolated polynucleotide and polynucleotide in isolated form. The polynucleotides may be of genomic, cDNA, RNA, semisynthetic, synthetic origin, or any combinations thereof. Thus, a substantially pure polynucleotide may contain at most about 10%, at most about 8%, at most about 6%, at most about 5%, at most about 4%, at most about 3%, at most about 2%, at most about 1%, or at most about 0.5% by weight of other polynucleotide material with which it is natively or recombinantly associated. A substantially pure polynucleotide may, however, include naturally occurring 5′ and 3′ untranslated regions, such as promoters and terminators. The substantially pure polynucleotide may be at least about 90% pure, at least about 92% pure, at least about 94% pure, at least about 95% pure, at least about 96% pure, at least about 97% pure, at least about 98% pure, at least about 99%, or at least about 99.5% pure by weight. The polynucleotides of the present invention may be in a substantially pure form. The polynucleotides disclosed herein may be in “essentially pure form”, i.e., that the polynucleotide preparation is essentially free of other polynucleotide material with which it is natively or recombinantly associated.

A subsequence refers to a nucleotide sequence having one or more nucleotides deleted from the 5′ and/or 3′ end of the full-length coding sequence or a homologous sequence thereof, wherein the subsequence encodes a polypeptide fragment having kinase activity. By way of example, a nucleotide sequence encoding the kinase domain of a BIN2 is a subsequence.

As used herein, the term “hybridizes under stringent conditions” is intended to describe conditions for hybridization and washing under which nucleotide sequences typically remain hybridized to each other. Such stringent conditions are known to those skilled in the art and can be found in Current Protocols in Molecular Biology (John Wiley & Sons. NY (1989)), 6.3.1-6.3.6. An example of stringent hybridization conditions is hybridization in 6× sodium chloride/sodium citrate (SSC) at about 45° C., followed by one or more washes in 0.2× SSC, 0.1% SDS at 50° C. Another example of stringent hybridization conditions is hybridization in 6× sodium chloride/sodium citrate (SSC) at about 45° C., followed by one or more washes in 0.2× SSC, 0.1% SDS at 55° C. A further example of stringent hybridization conditions is hybridization in 6× sodium chloride/sodium citrate (SSC) at about 45° C., followed by one or more washes in 0.2× SSC, 0.1% SDS at 60° C. Stringent hybridization conditions may also be hybridization in 6× sodium chloride/sodium citrate (SSC) at about 45° C., followed by one or more washes in 0.2× SSC, 0.1% SDS at 65° C. Moreover, stringency conditions (and the conditions that should be used if the practitioner is uncertain about what conditions should be applied to determine if a molecule is within a hybridization limitation of the invention) are 0.5M Sodium Phosphate, 7% SDS at 65° C.. followed by one or more washes at 0.2× SSC, 1% SDS at 65° C. An isolated nucleic acid molecule that hybridizes under stringent conditions to a kinase sequence of the invention may correspond to a naturally-occurring nucleic acid molecule.

Kinases and phosphatases play significant roles in the signaling pathways associated with cellular growth. For example, protein kinases are involved in the regulation of signal transmission from cellular receptors, e.g., growth-factor receptors, entry of cells into mitosis, and the regulation of cytoskeleton function, e.g., actin bundling. Also by way of example, phosphatases are involved in removing phosphate groups from proteins. The removal of a phosphate group may allow other proteins or molecules to bind. The removal of a phosphate group may terminate the kinase activity of a protein. The removal of a phosphate group may prevent other molecules or proteins from binding.

Assays for measuring kinase and/or phosphatase activity are well known in the art depending on the particular kinase and phosphatase. As used herein, “kinase protein activity”, “biological activity of a kinase protein”, or “functional activity of a kinase protein” refers to an activity exerted by a kinase protein, polypeptide, or nucleic acid molecule on a kinase-responsive cell as determined in vivo, or in vitro, according to standard assay techniques. A kinase activity can be a direct activity, such as autophosphorylation or an association with or an enzymatic activity on a second protein. As used herein, “phosphatase protein activity”, “biological activity of a phosphate protein”, or “functional activity of a phosphate protein” refers to an activity exerted by a phosphate protein, polypeptide, or nucleic acid molecule on a kinase-responsive cell as determined in vivo. or in vitro, according to standard assay techniques. A phosphate activity can be a direct activity, such as dephosphorylation of a serine, threonine or tyrosine phosphorylated residue.

The term “active fragment” or “functional fragment” as used herein refers to a polypeptide having one or more amino acids deleted from the amino and/or carboxyl terminus of a full-length polypeptide or a homologous sequence thereof, wherein the fragment retains kinase or phosphatase activity.

The present invention also provides for mutations in proteins that do not affect the activity of the protein. For example. conservative amino acid substitutions may be made at one or more predicted, nonessential amino acid residues such that the mutant retains its functional activity. A nonessential amino acid residue is a residue that can be altered from the wild-type sequence of a kinase protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues or for amino acid residues residing within a conserved protein domain, such as the serine/threonine protein kinase domain of the disclosed clones, where such residues are essential for protein activity.

Phosphatase Activity

The present invention relates to the identification of a novel class of phosphatase activity for the proteins of the BSU1 and PP1 phosphatase families. These novel activities remove phosphate residues from amino acids that have previously been phosphorylated. either by autophosphorylation, or by the activity of another protein, such as a kinase. The phosphatases may remove a phospho group from a serine, threonine or tyrosine amino acid.

The present invention provides for regulating cell signal transductions systems through introducing the BSU1 or PP1 or functional equivalents or homologs thereof phosphatase proteins into a cell or an in vitro solution comprising protein extract such as lysate. The phosphatase proteins may comprise the protein or functional equivalent or homologs of BSU1 or PP1. The phosphatase may be introduced or produced via a nucleic acid encoding the phosphatase or fragment thereof. The nucleic acid may comprise a vector.

The present invention provides for regulating signal transduction in a cell through the phosphatase activity of BSU1 or PP1. BSU1 may dephosphorylate the kinase BIN2, or functional equivalents thereof. The present invention further provides for regulating GSK3 in a cell, such as a eukaryotic cell. BSU1 may dephosphorylate GSK3. BSU1 may be introduced into a cell, such as through transfecting a nucleic acid encoding BSU1. BSU1 may be mutated and/or truncated, as discussed herein. PP1 may dephosphorylate the kinase BIN2, or functional equivalents thereof. The present invention further provides for regulating GSK3 in a cell, such as a eukaryotic cell. PP1 may dephosphorylate GSK3. PP1 may be introduced into a cell, such as through transfecting a nucleic acid encoding PP1. PP1 may be mutated and/or truncated, as discussed herein.

The activity of BSU1 or PP1 or functional equivalents or homologs thereof may affect signaling in an eukaryotic cell, such as a mammalian cell. The BSU1 or PP1 or functional equivalents or homologs thereof may regulate GSK3 kinase activity. As discussed herein, GSK3 may affect Wnt signaling, particularly via β-catenin. GSK3 also affects insulin signaling and neuron degeneration. GSK3 may be a target for the treatment of cancer, diabetes, and Alzheimer's disease. Accordingly, BSU1 or PP1 or functional equivalents or homologs thereof may affect Wnt signaling. BSU1 or PP1 or functional equivalents or homologs thereof may affect 13-catenin signaling. As discussed herein, GSK3 may be inhibited by Akt phosphorylation. Accordingly, BSU1 or PP1 or functional equivalents or homologs thereof may affect Akt signaling.

The present invention provides for determining and/or modulating phosphatase activity in a cell. The cell may be in an animal or part thereof. The cell may be in a plant or a part thereof, such as a root, stem, leaf, seed, flower. fruit, anther, nectary, ovary, petal, tapetum, xylem, or phloem. By way of example, plants include embryophytes, bryophytes, spermatophyes, nematophytes, tracheophytes, soybean, rice, tomato, alfalfa, potato, pea, grasses, herbs, trees, algae. mosses. fungi, vines, ferns, bushes, barley, wheat, hops, maize, lettuce, orange, peach, citrus, lemon. lime, coconut. palm, pine, oak, cedar, mango, pineapple, rhubarb, strawberry, blackberry, blackcurrant, blueberry. raspberry, kiwi, grape, rutabega, parsnip, sweet potato, turnip, mushroom (Fungus), pepper, cilantro, onion, leek, fennel, clove, avocado. or cucumber. It also includes biofuels crops such as Miscanthus or switchgrass, poplar, Sorghum, and Brachypodium.

Suitable host cells for expressing the phosphatases of the present invention in higher eukaryotes include: 293 (human embryonic kidney) (ATCC CRL-1573); 293F (Invitrogen, Carlsbad Calif.); 293T and derivative 293T/17(293tsA1609neo and derivative ATCC CRL-11268) (human embryonic kidney transformed by SV40 T antigen); COS-7 (monkey kidney CV1 line transformed by SV40)(ATCC CRL1651); BHK (baby hamster kidney cells) (ATCC CRL10); CHO (Chinese hamster ovary cells); mouse Sertoli cells; CVI (monkey kidney cells) (ATCC CCL70); VER076 (African green monkey kidney cells) (ATCC CRL1587); HeLa (human cervical carcinoma cells) (ATCC CCL2); MDCK (canine kidney cells) (ATCC CCL34); BRL3A (buffalo rat liver cells) (ATCC CRL1442); W138 (human lung cells) (ATCC CCL75); HepG2 (human liver cells) (HB8065); and MMT 060652 (mouse mammary tumor) (ATCC CCL51).

The invention also includes host cells transfected with a vector or an expression vector encoding the phosphatases of the invention, including prokaryotic cells, such as E. coli or other bacteria, or eukaryotic cells, such as yeast cells or animal cells. The living cell cultures may comprise prokaryotic cells or eukaryotic cells. Examples of sources for prokaryotic cells include but are not limited to bacteria or archaea. Examples of sources for eukaryotic cells include but are not limited to: yeast, fungi, protists, mammals, arthropods, humans, animals, molluscs, annelids, nematodes, crustaceans, platyhelminthes, monotremes, fish, marsupials, reptiles, amphibians, birds, rodents, insects, and plants.

The present invention provides nucleic acids encoding the phosphatases described herein, such as BSU1. The present invention also provides nucleic acids that encode polypeptides with conservative amino acid substitutions. The nucleic acids of the present invention may encode polypeptides that dephosphorylate BIN2 or GSK3 or variants thereof. The isolated nucleic acids may have at least about 60%, 70%, 80% 85%, 90%, 95%, or 99% sequence identity with BSU1. The isolated nucleic acids may encode a polypeptide having an amino acid sequence having at least about 80%, 85%, 90%, 95%, or 99% sequence identity to amino acid sequences associated with BSU1. The isolated nucleic acid may hybridize to the above identified nucleic acid sequences under stringent conditions and encode a poly peptide that dephosphorylates BIN2 or GSK3 or variants thereof.

The nucleic acids encoding the BSU1 or PP1. or functional equivalents or homologs thereof phosphatase proteins may be genetically fused to expression control sequences for expression. Suitable expression control sequences include promoters that are applicable in the target host organism. Such promoters are well known to the person skilled in the art for diverse hosts from prokaryotic and eukaryotic organisms and are described in the literature. For example, such promoters may be isolated from naturally occurring genes or may be synthetic or chimeric promoters. Likewise, the promoter may already be present in the target genome and may be linked to the nucleic acid molecule by a suitable technique known in the art, such as for example homologous recombination.

The present invention also provides expression cassettes for inserting the nucleic acid encoding a BSU1 or PP1 phosphatase into target nucleic acid molecules such as vectors or genomic DNA. For this purpose. the expression cassette is provided with nucleotide sequences at its 5′ and 3′-flanks facilitating its removal from and insertion into specific sequence positions like, for instance, restriction enzyme recognition sites or target sequences for homologous recombination as, e.g. catalyzed by recombinases.

The present invention also relates to vectors. particularly plasmids, cosmids, viruses and bacteriophages used conventionally in genetic engineering, that comprise a nucleic acid molecule or an expression cassette encoding BSU1, or PP1, or functional equivalents or homologs thereof.

In one embodiment of the invention, the vectors of the invention are suitable for the transformation of fungal cells, plant cells, cells of microorganisms (i.e. bacteria, protists, yeasts, algae etc.) or animal cells, in particular mammalian cells. Preferably, such vectors are suitable for the transformation of human cells. Methods which are well known to those skilled in the art can be used to construct recombinant vectors; see, for example, the techniques described in Sambrook and Russell, Molecular Cloning: A Laboratory Manual, CSH Press, 2001, and Ausubel, Current Protocols in Molecular Biology, Green Publishing Associates and Wiley Interscience, N.Y., 1989. Alternatively, the vectors may be liposomes into which the nucleic acid molecules or expression cassettes of the invention can be reconstituted for delivery to target cells. Likewise, the term “vector” refers to complexes containing such nucleic acid molecules or expression cassettes which furthermore comprise compounds that are known to facilitate gene transfer into cells such as polycations, cationic peptides and the like. The vector of the present invention contains nucleic acids encoding BSU1, or PP1, or functional equivalents, or homologs thereof.

In addition to the nucleic acid molecule or expression cassette of the invention, the vectors may contain further genes such as marker genes which allow for the selection of said vector in a suitable host cell and under suitable conditions. Generally, the vector also contains one or more origins of replication. The vectors may also comprise terminator sequences to limit the length of transcription beyond the nucleic acid encoding the biosensor fusion proteins. The nucleic acid molecules contained in the vectors may be operably linked to expression control sequences allowing expression, i.e. ensuring transcription and synthesis of a translatable RNA, in prokaryotic or eukaryotic cells.

For genetic engineering, e.g. in prokaryotic cells, the nucleic acid molecules of the invention or parts of these molecules can be introduced into plasmids which permit mutagenesis or sequence modification by recombination of DNA sequences. Standard methods (see Sambrook and Russell, Molecular Cloning: A Laboratory Manual, CSR Press, 2001) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions. deletions or substitutions are possible, in vitro mutagenesis, “primer repair”, restriction or ligation can be used. In general, sequence analysis, restriction analysis and other methods of biochemistry and molecular biology are carried out as analysis methods.

The present invention also provides for directed expression of nucleic acids encoding BSU1 phosphatase or homolog or functional equivalents thereof. It is known in the art that expression of a gene can be regulated through the presence of a particular promoter upstream (5′) of the coding nucleotide sequence. Tissue specific promoters for directing expression in a particular tissue in an animal are known in the art. For example, databases collect and share these promoters (Chen et al., Nucleic Acids Res. 34: D)04-D107, 2006). In plants, promoters that direct expression in the roots, seeds, or fruits are known.

The present invention further provides isolated polypeptides comprising a phosphatase BSU1 or PP1 or functional equivalents or homolgs thereof fused to additional polypeptides. The additional polypeptides may be fragments of a larger polypeptide. In one embodiment, there are one, two, three, four, or more additional polypeptides fused to the phosphatase. In some embodiments, the additional polypeptides are fused toward the amino terminus of the phosphatase. In other embodiments. the additional polypeptides are fused toward the carboxyl terminus of the phosphatase. In further embodiments, the additional polypeptides flank the phosphatase. In some embodiments, the nucleic acid molecules encode a fusion protein comprising nucleic acids fused to the nucleic acid encoding the phosphatase. The fused nucleic acid may encode polypeptides that may aid in purification and/or immunogenicity and/or stability without shifting the codon reading frame of the phosphatase. In some embodiments. the fused nucleic acid will encode for a poly peptide to aid purification of the phosphatase. In some embodiments the fused nucleic acid w ill encode for an epitope and/or an affinity tag. In other embodiments, the fused nucleic acid will encode for a polypeptide that correlates to a site directed for, or prone to, cleavage. In other embodiments. the fused nucleic acid will encode for polypeptides that are sites of enzymatic cleavage. In further embodiments. the enzymatic cleakage will aid in isolating the phosphatase.

In other embodiments, the multiple nucleic acids will be fused to the nucleic acid encoding the phosphatases. The fused nucleic acids may encode for polypeptides that aid purification and/or enzymatic cleavage and/or stability. In further embodiments. the fused nucleic acids will not elongate the expressed polypeptide significantly.

In some embodiments the additional polypeptides may comprise an epitope. In other embodiments, the additional polypeptides may comprise an affinity tag. By way of example, fusion of a polypeptide comprising an epitope and/or an affinity tag to a phosphatase may aid in purification and/or identification of the polypeptide. By way of example, the polypeptide segment may be a His-tag, a myc-tag, an S-peptide tag, a MBP tag (maltose binding protein), a GST tag (glutathione S-transferase), a FLAG tag, a thioredoxin tag, a GFP tag (green fluorescent protein), a BCCP (biotin carboxyl carrier protein), a calmodulin tag, a Strep tag, an HSV-epitope tag. a V5-epitope tag. and a CB P tag. The use of such epitopes and affinity tags is known to those skilled in the art.

In further embodiments. the additional polypeptides may provide a fusion protein comprising sites for cleavage of the polypeptide. The cleavage sites are useful for later cleaving the phosphatase from the fused polypeptides, such as with targeting polypeptides. As an example, a polypeptide may be cleaved by hydrolysis of the peptide bond. In some embodiments. the cleavage is performed by an enzyme. In some embodiments cleavage occurs in the cell. In other embodiments, cleavage occurs through artificial manipulation and/or artificial introduction of a cleaving enzyme. By way of example, cleavage enzymes may include pepsin, trypsin, chymotrypsin, and/or Factor Xa.

Fusion polypeptides may further possess additional structural modifications not shared with the same organically synthesized peptide, such as adenylation, carboxylation, glycosylation, hydroxylation, methylation. phosphorylation or myristylation. These added structural modifications may be further selected or preferred by the appropriate choice of recombinant expression system. On the other hand. fusion polypeptides may have their sequence extended by the principles and practice of organic synthesis.

Generally, the fusion proteins of the present invention containing BSU1 or PP1 or functional equivalents or homolgs thereof may be produced according to techniques: which are described in the prior art. For example, these techniques involve recombinant techniques which can be carried out as described in Sambrook and Russell. Molecular Cloning: A Laboratory Manual, CSH Press, 2001 or in Volumes 1 and 2 of Ausubel, Current Protocols in Molecular Biology, Current Protocols, 1994. Accordingly, the individual portions of the fusion protein may be provided in the form of nucleic acid molecules encoding them which are combined and, subsequently, expressed in a host organism or in vitro. Alternatively, the provision of the fusion protein or parts thereof may involve chemical synthesis or the isolation of such portions from naturally occurring sources, whereby the elements which may in part be produced by recombinant techniques may be fused on the protein level according to suitable methods, e.g. by chemical cross-linking for instance as disclosed in WO 94/04686. Furthermore, if deemed appropriate, the fusion protein may be modified post-translationally in order to improve its properties for the respective goal, e.g., to enhance solubility, to increase pH insensitivity, to be better tolerated in a host organism, to make it adherent to a certain substrate in vivo or in vitro, the latter potentially being useful for immobilizing the fusion protein to a solid phase etc. The person skilled in the art is well aware of such modifications and their usefulness. Illustrating examples include the modification of single amino acid side chains (e.g. by glycosylation, myristolation, phosphorylation, carbethoxylation or amidation). coupling with polymers such as polyethylene glycol, carbohydrates, etc. or with protein moieties, such as antibodies or parts thereof, or other enzymes etc.

The present invention further provides for directing the BSU1 or PP1 or functional equivalents or homolgs thereof to particular organs, cell types, or subcellular locations. The nucleic acid encoding the phosphatase may be fused to a nucleic acid encoding a targeting sequence. Targeting expression of proteins to a subcellular compartment such as the chloroplast. vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine during protein synthesis and processing where the encoded protein is ultimately compartmentalized.

The presence of a signal sequence may direct a polypeptide to either an intracellular organelle or subcellular compartment or for secretion to the apoplast. Many signal sequences are known in the art. See, for example, Becker et al., Plant Mol. Biol. 20:49 (1992); Close, P. S., Master's Thesis, Iowa State University (1993); Knox, C., et al., Plant Mol. Biol. 9:3-17 (1987); Lerner et al., Plant Physiol. 91:124-)29 (1989); Frontes et al., Plant Cell 3:483-496 (1991); Matsuoka et al., Proc. Natl. Acad. Sci. 88:834 (1991); Gould et al., J. Cell. Biol. 108:1657 (1989); Creissen et al., Plant J. 2:129 (1991); Kalderon, et al., Cell 39:499-509 (1984); Steifel, et al., Plant Cell 2:785-793 (1990).

The term “targeting signal sequence” refers to amino acid sequences, the presence of which in an expressed protein targets it to a specific subcellular localization. For example, corresponding targeting signals may lead to the secretion of the expressed phosphatase, e.g. from a bacterial host in order to simplify its purification. Preferably, targeting of the phosphatase may be used to affect the phosphatase activity, and/or the thereby affected GSK3/BIN2 activity, in a specific subcellular or extracellular compartment. Appropriate targeting signal sequences useful for different groups of organisms are known to the person skilled in the art and may be retrieved from the literature or sequence data bases.

The BSU1 or PP1 or functional equivalents or homolgs thereof of the present invention may be expressed in any location in the cell, including the cytoplasm, cell surface or subcellular organelles such as the nucleus, vesicles. ER, v acuole. etc. Methods and vector components for targeting the expression of proteins to different cellular compartments are well known in the art. Transport of protein to a subcellular compartment such as the chloroplast, vacuole, peroxisome, glyoxysome, cell wall or mitochondrion or for secretion into the apoplast, may be accomplished by means of operably linking a nucleotide sequence encoding a signal sequence to the 5′ and/or 3′ region of a gene encoding the phosphatase. Targeting sequences at the 5′ and/or 3′ end of the structural gene may determine during protein synthesis and processing where the encoded protein is ultimately, compartmentalized.

Targeting to the plastids of a plant cell may be achieved. For example. the following targeting signal peptides can for instance be used: amino acid residues 1 to 124 of Arabidopsis thaliana plastidial RNA polymerase (AtRpoT 3) (Plant Journal 17:557-561, 1999): the targeting signal peptide of the plastidic Ferredoxin:NADP+ oxidoreductase (FNR) of spinach (Jansen et al., Current Genetics 13: 517-522. 1988) in particular, the amino acid sequence encoded by the nucleotides -171 to 165 of the cDNA sequence disclosed therein; the transit peptide of the waxy protein of maize including or without the first 34 amino acid residues of the mature waxy protein (Klosgen et al., Mol. Gen. Genet. 217: 155-161, 1989); the signal peptides of the ribulose bisphosphate carboxylase small subunit (Wolter et al., PNAS 85: 846-850, 1988; Nawrath et al., PNAS 91: 12760-12764, 1994), of the NADP malat dehydrogenase (Gallardo et al., Planta 197: 324-332, 1995), of the glutathione reductase (Creissen et al., Plant J. 8: 167-175, 1995) or of the RI protein (Lorberth et al., Nature Biotechnology 16: 473-477, 1998).

Targeting to the mitochondria of plant cells may be accomplished by using the following targeting signal peptides: amino acid residues 1 to 131 of Arabidopsis thaliana mitochondrial RNA polymerase (AtRpoT 1) (Plant Journal 17: 557-561, 1999) or the transit peptide described by Braun (EMBO J. 11: 3219-3227, 1992).

Targeting to the vacuole in plant cells may be achieved by using the following targeting signal peptides: The N-terminal sequence (146 amino acids) of the patatin protein (Sonnewald et al., Plant J. 1: 95-106, 1991) or the signal sequences described by Matsuoka and Neuhaus (Journal of Exp. Botany 50: 165-174, 1999); Chrispeels and Raikhel (Cell 68: 613-6)6, 1992); Matsuoka and Nakamura (PNAS 88: 834-838, 1991); Bednarek and Raikhel (Plant Cell 3: 1195-1206, 1991) or Nakamura and Matsuoka (Plant Phys. 101: 1-5, 1993).

Targeting to he ER in plant cells may be achieved by using, e.g.. the ER targeting peptide HKTMLPLPLIPSELLSESSAEF (SEQ ID NO: 1) in conjunction with the C-terminal extension HDEL (Haselhoff, PNAS 94: 2122-2127, 1997). Targeting to the nucleus of plant cells may be achieved by using, e.g.. the nuclear localization signal (NLS) of the tobacco C2 polypeptide QPSLKRN/IKIQPSSQP (SEQ ID NO: 2).

Targeting to the extracellular space may be achieved by using e.g. one of the following transit peptides: the signal sequence of the proteinase inhibitor II-gene (Keil et al., Nucleic Acid Res. 14: 5641-5650, 1986; von Schaewen et al., EMBO J. 9: 30-33, 1990). of the lekansucrase gene from Envinia amylovora (Geier and Geider, Phys. Mol. Plant Pathol. 42: 387-404, 1993), of a fragment of the patatin gene B33 from Solarium tuberosuni, which encodes the first 33 amino acids (Rosahl et al., Mol Gen. Genet. 203: 214-220, 1986) or of the one described by Oshima et al. (Nucleic Acids Res. 18: 181, 1990).

Furthermore, targeting to the membrane may be achieved by using the N-terminal signal anchor of the rabbit sucrase-isomaltase (Hegner et al., J. Biol. Chem. 276: 16928-16933, 1992).

Targeting to the membrane in mammalian cells can be accomplished by using the N-terminal myristate attachment sequence MGSSKSK (SEQ ID NO: 3) or C-terminal prenylation sequence CaaX (SEQ ID NO: 4), where “a” is an aliphatic amino acid (i.e. Val, Leu or Ile) and “X” is any amino acid (Garabet, Methods Enzymol. 332: 77-87, 2001).

Additional targeting to the plasma membrane of plant cells may be achieved by fusion to a phosphatase, preferentially to the sucrose transporter SUT1 (Riesmeier, EMBO J. 11: 4705-4713, 1992). Targeting to different intracellular membranes may be achieved by fusion to membrane proteins present in the specific compartments such as vacuolar water channels (γTIP) (Karlsson, Plant J. 21: 83-90, 2000). MCF proteins in mitochondria (Kuan, Crit. Rev. Biochem. Mol. Biol. 28: 209-233, 1993), triosephosphate translocator in inner envelopes of plastids (Flugge, EMBO J. 8: 39-46, 1989) and photosystems in thylacoids.

Targeting to the golgi apparatus can be accomplished using the C-terminal recognition sequence K(X)KXX (SEQ ID NO: 5) where “X” is any amino acid (Garabet, Methods Enzymol. 332: 77-87. 2001

Targeting to the peroxisomes can be done using the peroxisomal targeting sequence PTS I or PTS II (Garabet, Methods Enzymol. 332: 77-87, 2001).

Targeting to the nucleus in mammalian cells can be achieved using the SV-40 large T-antigen nuclear localisation sequence PKKKRKV (SEQ ID NO: 6) (Garabet, Methods Enzymol. 332: 77-87, 2001).

Targeting to the mitochondria in mammalian cells can be accomplished using the N-terminal targeting sequence MSVLTPLLLRGLTGSARRLPVPRAKISL (SEQ ID NO: 7) (Garabet. Methods Enzymol. 332: 77-87. 2001).

In some embodiments, expression of the BSU1 or PP1 or functional equivalents or homolgs thereof phosphatase, or substrates thereof, may be targeted to particular tissue(s) or cell type(s). For example, a particular promoter may be used to drive transcription of a nucleic acid encoding the BSU1 or PP1 or functional equivalents or homolgs thereof phosphatase, or substrates thereof. A promoter is an array of nucleic acid control sequences that direct transcription of a nucleic acid. A promoter includes necessary nucleic acid sequences near the start site of transcription, such as, in the case of a polymerase H type promoter, a TATA element. A promoter also optionally includes distal enhancer or repressor elements, which can be located as much as several thousand base pairs from the start site of transcription. A constitutive promoter is a promoter that is active under most environmental and developmental conditions. An inducible promoter is a promoter that is active under environmental or developmental regulation. Any inducible promoter can be used, see, e.g., Ward et al., Plant Mol. Biol. 22:361-366, 1993. Exemplary inducible promoters include, but are not limited to, that from the ACEI system (responsive to copper) (Meft et al., Proc. Natl. Acad. Sci. USA 90:4567-4571, 1993; In2 gene from maize (responsive to benzenesulfonamide herbicide safeners) (Hershey et al., Mol. Gen. Genetics 227:229-237, 1991, and Gatz et al., Mol. Gen. Genetics 243:32-38, 1994) or Tet repressor from Tn10 (Gatz et al., Mol. Gen. Genetics 227:229-237, 1991). The inducible promoter may respond to an agent foreign to the host cell, see , e.g., Schena et al., PNAS 88: 10421-10425. 1991.

The promoter may be a constitutive promoter. A constitutive promoter is operably linked to a gene for expression or is operably linked to a nucleotide sequence encoding a signal sequence which is operably linked to a gene for expression. Many different constitutive promoters can be utilized in the instant invention. For example, in a plant cell, constitutive promoters include, but are not limited to, the promoters from plant viruses such as the 35S promoter from CaMV (Odell et al.. Nature 313: 810-812, 1985) and the promoters from such genes as rice actin (McElroy et al., Plant Cell 2: 163-171, 1990); ubiquitin (Christensen et al.. Plant Mol. Biol. 12:619-632, 1989, and Christensen et al., Plant Mol. Biol. 18: 675-689, 1992); pEMU (Last et al., Theor. Appl. Genet. 81:581-588, 1991); MAS (Velten et al., EMBO J. 3:2723-2730. 1984) and maize H3 histone (Lepetit et al., Mol. Gen. Genetics 231: 276-285, 1992 and Atanassova et al., Plant Journal 2(3): 291-300, 1992). Prokaryotic promoter elements include those which carry optimal −35 and −10 (Pribnow box) sequences for transcription by RNA polymerase in Escherichia coli. Some prokaryotic promoter elements may contain overlapping binding sites for regulatory repressors (e.g. the Lac, and TAC promoters, which contain overlapping binding sites for lac repressor thereby conferring inducibility by the substrate homolog IPTG). Examples of prokaryotic genes from which suitable promoter sequences may be obtained include E. coli lac, ara, and trp. Prokaryotic viral promoter elements of the present invention include lambda phage promote s (e.g. P_{RM} and P_{R}), T7 phage promoter elements, and SP6 promoter elements. Eukaryotic promoter vector elements of the invention include both yeast (e.g. GAL1, GAL10, CYC1) and mammalian (e.g. promoters of globin genes and interferon genes). Further eukaryotic promoter vector elements include viral gene promoters such as those of the SV40 promoter, the CMV promoter, herpes simplex thymidine kinase promoter, as well as any of various retroviral LTR promoter elements (e.g. the MMTV LTR). Other eukaryote examples include the hMTIIa promoters (e.g. U.S. Pat. No. 5,457,034), the HSV-1 4/5 promoter (e.g. U.S. Pat. No. 5,501,979), and the early intermediate HCMV promoter (WO 92/17581).

The promoter may be a tissue-specific or tissue-preferred promoters. A tissue specific promoter assists to produce the phosphatase exclusively, or preferentially, in a specific tissue. Any tissue-specific or tissue-preferred promoter can be utilized. In plant cells, for example but not by way of limitation, tissue-specific or tissue-preferred promoters include, a root-preferred promoter such as that from the phaseolin gene (Mural et al., Science 23: 476-482, 1983, and Sengupta-Gopalan et al., PNAS 82: 3320-3324, 1985); a leaf-specific and light-induced promoter such as that from cab or rubisco (Simpson et al., EMBO J. 4(11): 2723-2729. 1985. and Timko et al., Nature 318: 579-582, 1985); an anther-specific promoter such as that from LAT52 (Twell et al., Mol. Gen. Genetics 217: 240-245, 1989); a pollen-specific promoter such as that from Zm13 (Guerrero et al., Mol. Gen. Genetics 244: 161-168, 1993) or a microspore-preferred promoter such as that from apg (Twell et al., Sex. Plant Reprod. 6: 217-224, 1993).

Furthermore, the present invention relates to expression cassettes comprising the above-described nucleic acid molecule of the invention and operably linked thereto control sequences allowing expression in prokaryotic or eukaryotic cells.

In a further embodiment, the invention relates to a method for producing cells capable of expressing the phosphatases of the invention comprising genetically engineering cells with an above-described nucleic acid molecule, expression cassette or vector of the invention.

Another embodiment of the invention relates to host cells, in particular prokaryotic or eukaryotic cells, genetically engineered with an above-described nucleic acid molecule, expression cassette or vector of the invention, and to cells descended from such transformed cells and containing a nucleic acid molecule, expression cassette or vector of the invention and to cells obtainable by the above-mentioned method for producing the same.

The host cells may be are bacterial, fungal, insect, plant or animal host cells. In one embodiment, the host cell is genetically engineered in such a way that it contains the introduced nucleic acid molecule stably integrated into the genome. In another embodiment, the nucleic acid molecule can be expressed so as to lead to the production of the phosphatase of the present invention.

An overview of different expression systems is for instance contained in Methods in Enzymology 153: 385-516, 1987, in Bitter et al. (Methods in Enzymology 153: 516-544, 1987) and in Sawers et al. (Applied Microbiology and Biotechnology 46: 1-9, 1996), Billman-Jacobe (Current Opinion in Biotechnology 7: 500-4, 1996), Hockney (Trends in Biotechnology 12: 456-463, 1994), and Griffiths et al., (Methods in Molecular Biology 75: 427-440, 1997). An overview of yeast expression systems is for instance given by Hensing et al. (Antoine von Leuwenhoek 67: 261-279, 1995), Bussineau (Developments in Biological Standardization 83: 13-19, 1994), Gellissen et al. (Antoine van Leuwenhoek 62: 79-93, 1992), Fleer (Current Opinion in Biotechnology 3: 486-496, 1992), Vedvick (Current Opinion in Biotechnology 2: 742-745, 1991) and Buckholz (Bio/Technology 9: 1067-1072. 1991).

Expression vectors have been widely described in the literature. As a rule, they contain not only a selection marker gene and a replication origin ensuring replication in the host selected. but also a bacterial or viral promoter and, in most cases, a termination signal for transcription. Between the promoter and the termination signal, there is in general at least one restriction site or a polylinker which enables the insertion of a coding nucleotide sequence. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli. S. cerevisae) are sufficiently described in the literature. Promoters permitting a particularly high expression of a downstream sequence are for instance the T7 promoter (Studier et al., Methods in Enzymology 185: 60-89, 1990), lacUV5, trp, trp-lacUV5 (DeBoe et al., in Rodriguez and Chamberlin (Eds), Promoters, Structure and Function; Praeger, N.Y., 1982, p. 462-481; DeBoer et al., PNAS 80: 21-25, 1983), Ip1, rac (Boros et al., Gene 42: 97-100, 1986). Inducible promoters may be used for the synthesis of proteins. These promoters often lead to higher protein yields than do constitutive promoters. In order to obtain an optimum amount of protein, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (isopropyl-.beta.-D-thiogalactopyranoside) (DeBoer et al., PNAS 80: 21-25, 1983). Termination signals for transcription such as the SV40-poly-A site or the tk-poly-A site useful for applications in mammalian cells are also described in the literature. Suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL)) or pC1 (Promega).

The transformation of the host cell with a nucleic acid molecule or vector according to the invention can be carried out by standard methods, as for instance described in Sambrook and Russell. Molecular Cloning: A Laboratory Manual, CSH Press, 2001; Methods in Yeast Genetics, A Laboratory Course Manual, Cold Spring Harbor Laboratory Press, 1990). For example. calcium chloride transfection is commonly utilized for prokaryotic cells, whereas, e.g., calcium phosphate or DEAE-Dextran mediated transfection or electroporation may be used for other cellular hosts. The host cell is cultured in nutrient media meeting the requirements of the particular host cell used, in particular in respect of the pH value, temperature, salt concentration, aeration, antibiotics, vitamins, trace elements etc.

The phosphatases according to the present invention can be recovered and purified from recombinant cell cultures by methods including ammonium sulfate or ethanol precipitation, acid extraction, anion or cation exchange chromatography, phosphocellulose chromatography. hydrophobic interaction chromatography. affinity chromatography, hydroxylapatite chromography and lectin chromatography. A ligand or substrate, such as B1N2 or a GSK3, such as GSK3α and GSK3β, for the phosphatase of the present invention may by used for affinity purification or a fusion protein of the phosphatase may be purified by applying an affinity chromatography with a substrate or ligand to which the fused portion binds, such as an affinity tag. Protein refolding steps can be used, as necessary, in completing the configuration of the protein. Finally, high performance liquid chromatography (HPLC) can be employed for final purification steps.

Accordingly, a further embodiment of the invention relates to a method for producing the phosphatases of the invention comprising culturing the above-described host cells under conditions allowing the expression of said phosphatases and recovering said phosphatases from the culture. Depending on whether the expressed protein is localized in the host cells or is secreted from the cell, the protein can be recovered from the cultured cells and/or from the supernatant of the medium.

Modifications to BSU1 and PP1 1

The present invention provides for modifying the BSU1 or PP1 protein. As discussed herein, functional equivalents comprise truncations or modifications to the amino acid sequence of wild type BSU1 or PP1, wherein the resulting polypeptide retains the ability to dephosphorylate a substrate. such as BIN2 or GSK3 or a phosphorylated fragment thereof For example, a truncation of BSU1 or PP1 may comprise the catalytic domain.

The present invention provides a truncated BSU1 or PP1 polypeptide and nucleic acids encoding such a truncated polypeptide. A truncated molecule may be any molecule that comprises less than a full-length version of the molecule. Truncated molecules provided by the present invention may include truncated biological polymers, and in one embodiment of the invention such truncated molecules may be truncated nucleic acid molecules or truncated polypeptides. Truncated nucleic acid molecules have less than the full-length nucleotide sequence of a known or described nucleic acid molecule. Such a known or described nucleic acid molecule may be a naturally occurring. a synthetic, or a recombinant nucleic acid molecule. so long as one skilled in the art would regard it as a full-length molecule. Thus, for example, truncated nucleic acid molecules that correspond to a gene sequence contain less than the full length gene where the gene comprises coding and non-coding sequences, promoters, enhancers and other regulatory sequences, flanking sequences and the like, and other functional and non-functional sequences that are recognized as part of the gene. In another example, truncated nucleic acid molecules that correspond to a mRNA sequence contain less than the full length mRNA transcript, which may include various translated and non-translated regions as well as other functional and non-functional sequences.

Mutations to the BSU1 or PP1 phosphatase may alter the phosphatase activity of the protein. The present invention also provides for mutations to the amino acid sequence of BSU1 or PP1, wherein the mutations affect the ability to dephosphorylate a substrate, such as B1N2 or GSK3, or phosphorylated fragments thereof. The mutations may be directed to nucleic acids encoding the BSU1 or PP1 phosphatases. The mutations may be directed to ensuring that the BSU1 or PP1 phosphatase or functional equivalents thereof are constitutively active. A constitutively active may be of use for providing increased growth or for ensuring that GSK3 or BIN2 phosphorylation is reduced. The mutations may also conversely be directed at providing a BSU1 or PP1 phosphatase or a functional equivalent thereof that cannot dephosphorylate GSK3 or B1N2. An inactive mutant may be of use for increasing GSK3 or BIN2 activity, or for reducing growth.

A mutation to BSU1 or PP1 or a functional equivalent thereof may be located in the catalytic domain of the phosphatase. The mutation may be at the active cysteine in the catalytic domain. The mutation may be at a conserved aspartate residue in the catalytic domain. The aspartate may be at position 510 of wild type BSU1. The present invention provides for mutations in which the aspartate residue in the catalytic domain of the phosphatase is replaced with an amino acid which does not cause significant alteration of the Kₘ of the enzyme (that is, does not cause a statistically significant increase or decrease of the Kₘ) but which results in a reduction in K_{cat}, such as to a rate of less than 1 per minute. Replacement of the wild type aspartate residue may result in a reduction of K_{cat} such that the K_{cat} of the substrate trapping mutant is less than 1 per minute, which is a reduction in K_{cat} compared with the wild type phosphatase. As understood by persons skilled in the art, the Michaelis constant Kₘ is a term that indicates a measure of the substrate concentration required for effective catalysis to occur and is the substrate concentration at which the reaction is occurring at one-half its maximal rate (½Vₘₐₓ). The K_{cat} of an enzyme provides a direct measure of the catalytic production of product under optimum conditions (particularly, saturated enzyme). The reciprocal of K_{cat} is often referred to as the time required by an enzyme to “turn over” one substrate molecule, and K_{cat} is sometimes called the turnover number. Vmax and Kcat are directly proportional; therefore, if, for example, K_{cat} of a substrate trapping mutant is reduced by 10⁴ compared to the K_{cat} of the wildtype enzyme, Vₘₐₓ is also decreased by a factor of 10⁴. These substrate trapping mutant phosphatases retain the ability to form a complex with, or bind to, their tyrosine phosphorylated substrates, but are catalytically attenuated (i.e., a substrate trapping mutant phosphatase retains a similar Km to that of the corresponding wildtype phosphatase, but has a Vmax which is reduced by a factor of at least 10²-10⁵ relative to the wildtype enzyme, depending on the activity of the wildtype enzyme relative to a K_{cat} of less than 1 min⁻¹). This attenuation includes catalytic activity that is either reduced or abolished relative to the wildtype phosphatase. For example, the aspartate residue can be changed or mutated to an alanine, valine, leucine, isoleucine, proline, phenylalanine, tryptophan, methionine, glycine, serine, threonine, cysteine, tyrosine, asparagine, glutamine, lysine, arginine or histidine.

Methods for Determining Phosphatase Activity

The present invention provides methods for identifying proteins that interact with BSU1 or PP1 or functional equivalents or homolgs thereof. The interacting proteins or chemical compounds may be substrates for BSU1 or PP1 or functional equivalents or homolgs thereof or may bind to BSU1 or PP1 or functional equivalents or homolgs thereof to affect the ability of the phosphatase to bind a substrate or to dephosphorylate a substrate. These methods may comprise providing a phosphatase to a cell or extract of the cell. The phosphatase may be encoded by a nucleic acid. The phosphatase may be a wild type or a mutant. such as a dominant negative mutant or a constitutively active mutant. The methods may further comprise introducing a substrate. The methods may also include a control such as a positive or a negative control, wherein a comparison of phosphatase activity or phosphatase binding/interaction can be made. For example, comparison with the demonstrated BSU1-BIN2 (or GSK3) or PP1-GSK3 (or BIN2) contained herein may function as a control.

Substrates may be identified through substrate trapping. Substrate trapping mutant phosphatases contain mutations in which the catalytic domain invariant aspartate and at least one tyrosine residue are replaced, wherein the tyrosine is replaced with an amino acid that is not capable of being phosphorylated, The amino acid that is not capable of being phosphorylated may include alanine, cysteine, aspartic acid, glutamine, glutamic acid. phenylalanine, glycine, histidine, isoleucine. lysine, leucine, methionine, asparagine, proline, arginine, valine or tryptophan. The desirability of the tyrosine replacement derives from the observation that under certain conditions in vivo, a phosphatase enzyme may itself undergo tyrosine phosphorylation in a manner that can alter interactions between the phosphatase and other molecules, including phosphatase substrates.

Substrates of BSU1 or PP1, may include full length tyrosine phosphorylated proteins and polypeptides as well as fragments (e.g., portions), derivatives or analogs thereof that can be phosphorylated at a tyrosine residue and that may, in certain embodiments, also be able to undergo phosphorylation at a serine or a threonine residue. For example, the substrate may be a tyrosine phosphorylated GSK3 or BIN2. Such fragments, derivatives and analogs include any naturally occurring or artificially engineered BSU1 or homolog thereof substrate polypeptide that retains at least the biological function of interacting with a BSU1 or homolog thereof as provided herein, for example by forming a complex with the BSU1 or homolog thereof. A fragment, derivative or analog of a BSU1 or homolog thereof substrate polypeptide, including substrates that are fusion proteins, may be: one in which one or more of the amino acid residues are substituted with a conserved or non-conserved amino acid residue, and such substituted amino acid residue may or may not be one encoded by the genetic code; one in which one or more of the amino acid residues includes a substituent group; one in which the substrate polypeptide is fused with another compound, such as a compound to increase the half-life of the polypeptide (e.g., polyethylene glycol) or a detectable moiety such as a reporter molecule; or, one in which additional amino acids are fused to the substrate polypeptide, including amino acids that are employed for purification of the substrate polypeptide or a proprotein sequence. Such fragments, derivatives and analogs are deemed to be within the scope of those skilled in the art.

BSU1 or PP1 or functional equivalents or homolgs thereof variants may be tested for enzymatic activity using any suitable assay for phosphatase activity, such as assays for PP1 or PP2. Such assays may be performed in vitro or within a cell-based assay. The assay may be performed with a pre-phosphorylated substrate. For example, ³²P-radiolabeled substrate may be used for the kinase reaction, resulting in radiolabeled, activated phosphatase substrate. A BSU1 or homolog thereof polypeptide may then be tested for the ability to dephosphorylate the substrate by contacting the BSU1 or homolog thereof polypeptide with the substrate under suitable conditions (e.g., Tris, pH 7.5,) mM EDTA, 1 mM dithiothreitol, 1 mg/mL bovine serum albumin for 10 minutes at 30° C.). Dephosphorylation of the substrate may be detected using any of a variety of assays, such as a coupled kinase assay (evaluating phosphorylation of the substrate using any assay generally known in the art) or direct)y, based on (1) the loss of radioactive phosphate groups (e.g., by gel electrophoresis, followed by autoradiography); (2) the shift in electrophoretic mobility following dephosphorylation; (3) the loss of reactivity with an antibody specific for phosphotyrosine, phosphoserine, or phosphothreonine or an antibody specific to the phosphorylated form of the substrate, for example, a phospho-GSK3α (Y279) antibody or phospho-GSK3f3 (Y216) antibody; or (4) a phosphoamino acid analysis of the substrate, such as with tandem mass spectrometry and liquid chromatography.

GSK3

The present invention further provides methods for identifying proteins that regulate kinases related to BIN2, such as GSK3(glycogen synthase 3 kinase). GSK3 (also Shaggy (Zeste White 3) in Drosophila) is a homolog for BIN2. 9P| may dephosphorylate GSK3 or functional equivalents thereof. BSU1 may dephosphorylate GSK3 or functional equivalents thereof. GSK3 is a proline-directed serine/threonine kinase originally identified as an activity that phosphorylates glycogen synthase as described in Woodgett, Trends Biochem Sci. 16:177-181 (199)). The role of GSK3 in glucose metabolism has since been elaborated. GSK3 consists of two isoforms, α and β, and is constitutively active in resting cells, inhibiting glycogen synthase by direct phosphorylation. Upon stimulation of certain pathways, such as via insulin activation, GSK3 is inactivated, thereby allowing the activation of glycogen synthase and possibly other insulin-dependent events. GSK3 is inactivated by other growth factors or hormones that. like insulin, signal through receptor tyrosine kinases. Examples of such signaling molecules include IGF-1 and EGF as described in Saito et al., Biochem. J. 303:27-31 (1994), Welsh et al., Biochem. J. 294:625-629 (1993), and Cross et al., Biochem. J. 303:21-26 (1994). GSK3 has been shown to phosphorylate β-catenin as described in Peifer et al., Develop. Biol. 166:543-56 (1994). Other activities of GSK3 in a biological context include GSK3's ability to phosphorylate tau protein in vitro as described in Mandelkow and Mandelkow, Trends in Biochem. Sci. 18:480-83 (1993), Mulot et al., FEBS Lett 349: 359-64 (1994), and Lovestone et al., Curr. Biol. 4:1077-86 (1995), and in tissue culture cells as described in Latimer et al., FEBS Lett 365:42-6 (1995). GSK3 may be involved in conditions such as Alzheimer's, bipolar, Huntington's, schizophrenia, diabetes, neurodegenerative disorders (chronic and acute), hair loss, and sperm immotility. In Alzheimer's, over activity of GSK3 may cause tau (τ) hyper-phosphorylation, increased β-amyloid production and local plaque-associated microglial-mediated inflammatory responses. GSK3s may work in the Wnt signaling pathway to phosphorylate β-catenin. Phosphorylation leads to ubiquitination and degradation by cellular proteases, thereby preventing it from entering the nucleus and activating transcription factors. For example, in fruit flies, when the protein Disheveled is activated by Wnt signaling, GSK3 is inactivated, thereby allowing β-catenin to accumulate and effect transcription of Wnt target genes. GSK3 may also phosphorylate Ci in the Hedgehog (Hh) signaling pathway, targeting it for proteolysis to an inactive form.

GSK3 has many other substrates. However, GSK3 is unusual among the kinases in that it usually requires a “priming kinase” to first phosphorylate a substrate, and then, only when the priming kinase has done its job can GSK3 additionally phosphorylate the substrate. The consequence of GSK3 phosphorylation is usually inhibition of the substrate. For example, when GSK3 phosphorylates another of its substrates, the NFAT and BZR1/2 families of transcription factors, these transcription factors cannot translocate to the nucleus and are therefore inhibited. In addition to its important role in the Wnt signaling pathway, which is required for establishing tissue patterning during development, GSK3 is also critical for the protein synthesis that is induced in settings such as skeletal muscle hypertrophy. Its roles as an NFAT kinase also places it as a key regulator of both differentiation and cellular proliferation.

GSK3 can be inhibited by Akt phosphorylation, which can be part of insulin signal transduction. Accordingly. Akt is an activator of many of the signaling pathways blocked by GSK3. For example, in the setting of induced Akt signaling, it can be shown that NFAT is dephosphorylated. Furthermore, cytokine-dependent GSK3 phosphorylation in hemopoietic cells may regulate growth, and the PKC family of kinases may affect GSK3 phosphorylation.

As discussed above, GSK3, like BIN2, is cons itutively active. Accordingly, the present invention provides for identifying further eukaryotic homologs to BSU1 or PP1. The methods include sequence alignment and/or competition/comparison assays with BSU1 and/or PP1.

Methods of Treatment

The present invention further provides methods for treating diseases and/or conditions related to BIN2 or GSK3 activity comprising contacting a cell of a plant or animal with BSU1 or PP1 or functional equivalents or homolgs thereof or an aaent that modulates the activity of BSU1 or PP1, wherein increasing the phosphatase activity in the cell by either increasing BSU1 or PP1 or functional equivalents or homolgs thereof phosphatase expression and/or enzymatic activity increases dephosphorylation of GSK3 or BIN2. As used herein, the term “treatment” includes the application or administration of a therapeutic agent, such as BSU1 or PP1 or functional equivalents or homologs thereof, to a subject or to an isolated tissue or cell line from a subject, who is afflicted with amyloidosis, a symptom of amyloidosis or a predisposition toward amyloidosis, with the goal of curing, healing, alleviating, relieving, altering, remedying, ameliorating, improving or affecting the disease, the symptoms of disease or the predisposition toward disease.

In plants, for example, overactive BIN2 may result in changes to growth and sterility in the plant. The BSU1 or PP1 or functional equivalents or homolgs thereof or an agent that modulates the activity of BSU1 or PP1 may further aid a plant in recovering from a pathogen attack or preventing a pathogen attack. A pathogen may include fungi, bacertia, oocmycetes, virus, nematodes, protozoa, phytoplasmas and spiroplasmas, and parastici plant. A fungus may include, but is not limited to, ascomycetes, such as Fusarium, Thielaviopsis, Verticillium, Magnaporthe grisae, and basidiomycetes, such as Rhizoctonia, Phakospora, and Puccinia. Oomycetes may include, but is not limited to, Phytophthora and Pythium. Bacteria may include, but are not limited to, Burkholderia, Proteobacteria, such as Xanthomonas and Pseudomonas. Nematodes may include, but are not limited to, rrot knot nematodes, Globerodera, and cyst nematodes. The BSU1 or PP1 or functional equivalents or homolgs thereof or an agent that modulates the activity of BSU1 or PP1 may aid a plant to prevail in testing environmental conditions, such as impacted soil, frost, drought, flooding, nutrient deficiency, salt deposition, wind, fire, lightning, pollution (air and soil), herbicides, as well as interference by human's such as cultivation or vanda)ism.

In animals. overactive GSK3 may result in neurdegenerative disorders, such as Alzheimer's bipolar disorders, and schizophrenia; CNS disorders, such as multiple sclerosis; ischemic brain injury and/or stroke, traumatic brain injury; diabetes; alopecia; and. fertility. The BSU1or PP1 or functional equivalents or homolgs thereof or an agent that modulates the activity of BSU1 or PP1 may be used for the diagnosis and/or treatment of diseases, disorders. damage or injury of the brain and/or nervous system. Nervous system disorders that can be treated with the compositions of the invention (e.g., BSU1 or PP1 or functional equivalents or homolgs thereof or an agent that modulates the activity of BSU1 or PP1 of the invention), limited to nervous systems include, but are not limited injuries, and diseases or disorders which result in either a disconnection of axons, a diminution or degeneration of neurons, ordemyelination. Nervous system lesions which may be treated in a patient (including human and non-human mammalian patients) according to the methods of the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction orischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from nervous system tissue; (4) infectious lesions in which a portion of the nervous system is destroyed or injured as a result of infection, for example, by an abscess or associated with infection by human immunodeficiency virus, herpes zoster, or herpes simplex virus or with Lyme disease, tuberculosis, or syphilis; (5) degenerative lesions, in which a portion of the nervous system is destroyed or injured as a result of a degenerative process including but not limited to. degeneration associated with Parkinson's disease, Alzheimer's disease, Huntington's chorea, or amyotrophic lateral sclerosis (ALS): (6) lesions associated with nutritional diseases or disorders, in which a portion of the nervous system is destroyed or injured by a nutritional disorder or disorder of metabolism including, but not limited to vitamin B 12 deficiency, folic acid deficiency, Wernicke disease. tobacco-alcohol amblyopic, Marchiafava-Blanami disease (primary degeneration of the corpus callosum). and alcoholic cerebral degeneration; (7) neurological lesions associated with systemic diseases including, but not limited to diabetes (diabetic neuropathy, Bell's palsy), systemic lupuserythematosus, carcinoma, or sarcoidoisis; (8) lesions caused by toxic substances including alcohol, lead, or particular. neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy. and central pontine myelinolysis.

In one embodiment, the BSU1 or PP1 or functional equivalents or homolgs thereof or an agent that modulates the activity of BSU1 or PP1 of the invention are used to protect neural cells from the damaging effects of hypoxia. In a further preferred embodiment. the BSU1 or PP1 or functional equivalents or homolgs thereof or an agent that modulates the activity of BSU1 or PP1 of the invention are used to protect neural cells from the damaging effects of cerebral hypoxia.

In specific embodiments, motor neuron disorders that may be treated according to the invention include, but are not limited to, disorders such as infarction, infection, exposure to toxin, trauma, surgical damage, degenerative disease or malignancy that may affect motor neurons as well as other components of the nervous system, as well as disorders that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motor sensory Neuropathy (Charcot-Marie-Tooth Disease).

Further. BSU1 or PP1 or functional equivalents or homolgs thereof or an agent that modulates the activity of BSU1 or PP1 of the invention may play a role in neuronal survival: synapse formation; conductance; neural differentiation, etc. Thus, compositions of the invention (including BSU1 or PP1 or functional equivalents or homolgs thereof or an agent that modulates the activity of BSU1 or PP1) may be used to diagnose and/or treat or prevent diseases or disorders associated with these roles, including, but not limited to, learning and/or cognition disorders. The compositions of the invention may also be useful in the treatment or prevention of neurodegenerative disease states and/or behavioral disorders. Such neurodegenerative disease states and/or behavioral disorders include, but are not limited to, Alzheimer's Disease, Parkinson's Disease, Huntington's Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception.

Examples of neurologic diseases which can be treated or detected with BSU1 or PP1 or functional equivalents or homolgs thereof or an agent that modulates the activity of BSU1 or PP1 of the invention include, brain diseases, such as metabolic brain diseases which includes phenylketonuria such as maternal phenylketonuria, pyruvate carboxylase deficiency, pyruyate dehydrogenase complex deficiency, Wernicke's Encephalopathy. and brain edema,.

Additional neurologic diseases which can be treated or detected with BSU1 or PP1 or functional equivalents or homolgs thereof or an agent that modulates the activity of BSU1 or PP1 of the invention include dementia such as AIDS Dementia Complex, presenile dementia such as Alzheimer's Disease and Creutzfeldt-Jakob Syndrome, senile dementia such as Alzheimer's Disease and progressive supranuclear palsy, vascular dementia such as multi-infarct dementia, encephalitis (bacterial and viral), meningitis (bacterial and viral), and neoplasms of the central nervous system.

As used herein, “therapeutically effective amount” refers to that amount of the agent or compound which, when administered to a subject in need thereof, is sufficient to effect treatment. The amount of agent or compound which constitutes a “therapeutically effective amount” will vary depending on the severity of the condition or disease, and the age and body weight of the subject to be treated, but can be determined routinely by one of ordinary skill in the art having regard to his/her own knowledge and to this disclosure.

Pharmaceutical Compositions

Another aspect of the invention is directed toward the use of BSU1 or PP1 or functional equivalents or homologs thereof as part of a pharmaceutical composition. The present invention also comprises administering to a plant or an animal or a cell of a plant or a cell of an animal an agent that modulates BSU1 activity on BIN2 and administering to an animal or a cell thereof an agent that modulates PP1 activity on GSK3. The nucleic acids of the present invention may also be used as part of a pharmaceutical composition. The compositions used in the methods of the invention generally comprise, by way of example and not limitation, an effective amount of a nucleic acid or polypeptide of the invention or antibody of the invention. The nucleic acids and polypetides of the invention may further comprise pharmaceutically acceptable carriers, excipients, or stabilizers known in the art (see generally Remington, (2005) The Science and Practice of Pharmacy, Lippincott, Williams and Wilkins).

The nucleic acids and polypeptides of the present invention may be in the form of lyophilized formulations or aqueous solutions. Acceptable carriers, excipients, or stabilizers may be nontoxic to recipients at the dosages and concentrations that are administered. Carriers, excipients or stabilizers may further comprise buffers. Examples of buffers include. but are not limited to, carbohydrates (such as monsaccharide and disaccharide), sugars (such as sucrose, mannitol, and sorbitol), phosphate, citrate, antioxidants (such as ascorbic acid and methionine), preservatives (such as phenol, butanol, benzanol; alkyl parabens, catechol, octadecyldimethylbenzyl ammonium chloride, hexamethoniuni chloride, resorcinol, cyclohexanol, 3-pentanol, benzalkonium chloride, benzethonium chloride, and m-cresol), low molecular weight polypeptides, proteins (such as serum albumin or immunoglobulins), hydrophilic polymers amino acids, chelating agents (such as EDTA), salt-forming counter-ions, metal complexes (such as Zn-protein complexes), and non-ionic surfactants (such as TWEEN™ and polyethylene glycol).

The nucleic acids and polypeptides of the present invention may be administered to a patient in need thereof using standard administration protocols. For instance, the BSU1 and PP1 phosphatase proteins of the present invention can be provided alone, or in combination. or in sequential combination with other agents that modulate a particular pathological process. As used herein, two agents are said to be administered in combination when the two agents are administered simultaneously or are administered independently in a fashion such that the agents will act at the same or near the same time.

The agents of the present invention can be administered via parenteral, subcutaneous, intravenous, intramuscular, intraperitoneal, transdermal and buccal routes. For example, an agent may be administered locally to a site of injury via microinfusion. Alternatively. or concurrently, administration may be noninvasive by either the oral, inhalation, nasal, or pulmonary route. The dosage administered will be dependent upon the age, health. and weight of the recipient. kind of concurrent treatment. if any, frequency of treatment, and the nature of the effect desired.

The present invention further provides compositions containing one or more nucleic acids and polypeptides of the present invention. While individual needs vary, determination of optimal ranges of effective amounts of each component is within the skill of the art. Typical dosages comprise about 1 pg/kg to about 100 mg/kg body weight. The preferred dosages for systemic administration comprise about 100 ng/kg to about 100 mg/kg body weight or about 100-200 mg of protein/dose. The preferred dosages for direct administration to a site via microinfusion comprise about I ng/kg to about 1 mg/kg body weight. When administered via direct injection or microinfusion, nucleic acids and polypeptides of the present invention may be engineered to exhibit reduced or no binding of iron to prevent, in part, localized iron toxicity.

In addition to the pharmacologically nucleic acids and polypeptides of the present invention, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient. Suitable formulations for oral administration include hard or soft gelatin capsules, pills. tablets, including coated tablets, elixirs, suspensions, syrups or inhalations and controlled release forms thereof.

In practicing the methods of this invention, the agents of this invention may be used alone or in combination, or in combination with other therapeutic or diagnostic agents. In certain preferred embodiments, the compounds of this invention may be co-administered along with other compounds typically prescribed for these conditions according to generally accepted medical practice. The compounds of this invention can be utilized in vivo, ordinarily in mammals, such as humans, sheep, horses, cattle. pigs, dogs, cats, rats and mice, or in vitro.

The pharmaceutical composition of the present invention can further comprise additional agents that serve to enhance and/or complement the desired effect. By way of example, to enhance the immunogenicity of BSU1 or PP1 or functional equivalents or homolgs thereof of the invention or BIN2 or GSK3 or functional equivalents or homologs thereof being administered as a subunit vaccine, the pharmaceutical composition may further comprise an adjuvant.

Methods for Identifying Modulators of Phosphatase Activity

In one aspect of the present invention, BSU1 or PP1 or functional equivalents or homologs thereof may be used to identify agents that modulate the phosphatase activity of BSU1or PP1 or functional equivalents or homologs thereof. Such agents may inhibit or enhance signal transduction via a kinase cascade, leading to altered gene transcription. For example, inhibited DSU) or PP1 or functional equivalents or homologs thereof will allow GSK3 and/or B1N2 signaling to proceed in an increased manner, thereby increasing NFAT or BZR1/2 phosphorylation and inhibiting gene transcription. An agent that modulates phosphatase activity of BSU1 or PP1 or functional equivalents or homologs thereof may alter expression and/or stability of the phosphatase, phosphatase protein activity and/or the ability of the phosphatase to dephosphorylate a substrate. Agents that may be screened within such assays include, but are not limited to, antibodies and antigen-binding fragments thereof. competing substrates or peptides that represent, for example. a catalytic site or a dual phosphorylation motif, antisense polynucleotides and ribozymes that interfere with transcription and/or translation of BSU1or a homolog thereof and other natural and synthetic molecules, for example small molecule inhibitors, that bind to and inactivate BSU1 or PP1 or functional equivalents or homologs thereof.

Candidate agents for use in a method of screening for a modulator of phosphatase activity of BSU1 or PP1 or functional equivalents or homologs thereof according to the present invention may be provided as “libraries” or collections of compounds, compositions or molecules. Such molecules typically include compounds known in the art as “small molecules” and having molecular weights less than 10⁵ Daltons, less than 10⁴ Daltons, or less than 10³ Daltons. For example, members of a library of test compounds can be administered to a plurality of samples, each containing at least one BSU1 or homolog thereof phosphatase polypeptide as described herein, and then assayed for their ability to enhance or inhibit BSU1 or homolog thereof phosphatase dephosphorylation of, or binding to, a substrate. Compounds so identified as capable of influencing BSU1 or PP1 or functional equivalents or homologs thereof phosphatase function (e.g., phosphotyrosine and/or phosphoserine/threonine dephosphorylation) are valuable for therapeutic and/or diagnostic purposes, since they permit treatment and/or detection of diseases associated with BSU1 or PP1 or functional equivalents or homologs thereof phosphatase activity, as well as the treatment and/or detection of diseases associated with GSK3 and/or BIN2 activity. Such compounds are also valuable in research directed to molecular signaling mechanisms that involve BSU1 or PP1 or functional equivalents or homologs thereof, and to refinements in the discovery and development of future BSU1 or PP1 or functional equivalents or homologs thereof compounds exhibiting greater specificity.

The present invention also provides for identifying compounds that modulate the phosphatase activity of BSU1 or PP1 or functional equivalents or homologs thereof from a combinatorial library. The candidate agents further may be provided as members of a combinatorial library, which may include synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/U391/08694, PCT/US91/04666, which are hereby incorporated by reference in their entireties) or other compositions that may include small molecules as provided herein (see e.g., PCT/US94/08542, EP 0774464, U.S. Pat. No. 5,798,035, U.S. Pat. No. 5,789,172. U.S. Pat. No. 5,751,629, which are hereby incorporated by reference in their entireties). Those having ordinary skill in the art will appreciate that a diverse assortment of such libraries may be prepared according to established procedures, and tested using BSU1 or homolog thereof according to the present disclosure.

The present invention also provides for identifying modulating agents. Modulating agents may be identified by combining a candidate agent with a BSU1 or PP1 or functional equivalents or homologs thereof phosphatase polypeptide or a polynucleotide encoding such a polypeptide, in vitro or in vivo, and evaluating the effect of the candidate agent on the phosphatase activity, such as through the use of a phosphatase assay. An increase or decrease in phosphatase activity can be measured in the presence and absence of a candidate agent. For example, a candidate agent may be included in a mixture of active phosphatase polypeptide and substrate (e.g., BIN2 or GSK3), with or without pre-incubation with one or more components of the mixture. The effect of the agent on phosphatase activity may then be evaluated by quantitating the loss of phosphate from the substrate. and comparing the loss with that achieved without the addition of a candidate agent. Alternatively, a coupled kinase assay may be used, in which phosphatase activity is indirectly measured based on downstream kinase activity, such as GSK3 or BIN2 kinase activity.

Alternatively, a polynucleotide comprising a BSU1 or PP1 promoter operably linked to a BSU1 or PP1 coding region or reporter gene may be used to evaluate the effect of a test compound on BSU1 or PP1 transcription. Such assays may be performed in cells that express BSU1 or PP1 endogenously or in cells transfected with an expression vector comprising a BSU1 or PP1 promoter linked to a reporter gene. The effect of a test compound may then be evaluated by assaying the effect on transcription of BSU1 or PP1 or the reporter using, for example, a Northern blot analysis, renilla/luciferase or other suitable reporter activity assay.

Phosphatase activity may also be measured in whole cells transfected with a reporter gene whose expression is dependent upon the activation or inactivation of an appropriate substrate. For example, cells expressing the phosphatases of the present invention may be transfected with a substrate-dependent promoter linked to a reporter gene. For example, as disclosed herein, BIN2 and GSK3 proteins phosphorylate BZR1/2 and NFAT transcription factors, which may therefore be incorporated into a reporter system. In such a system, expression of the reporter gene (which may be readily detected using methods well known to those of ordinary skill in the art) depends upon the activity of the substrate of the phosphatase. Dephosphorylation of substrate may be detected based on changes in reporter activity. Candidate modulating agents may be added to such a system, as described above, to evaluate their effect on phosphatase activity.

The present invention further provides methods for identifying a molecule that interacts with, or binds to, BSU1 or PP1 or functional equivalents or homologs thereof. Such a molecule generally associates with BSU1 or PP1 or functional equivalents or homologs thereof with an affinity constant (Kₐ) of at least about 10⁴, at least about 10⁵, at least about 10⁶, at least about 10⁷ or at least about 10⁸. Affinity constants may be determined using well known techniques. Methods for identifying interacting molecules may be used, for example, as initial screens for modulating agents, or to identify factors that are involved in the in vivo phosphatase activity. Techniques for substrate trapping, as described above, are also contemplated according to certain embodiments provided herein. In addition to standard binding assays, there are many other techniques that are well known for identifying interacting molecules, including yeast two-hybrid screens, phage display and affinity techniques. Such techniques may be performed using routine protocols, which are well known to those having ordinary skill in the art. Within these and other techniques, candidate interacting proteins, such as phosphatase substrates, may be phosphorylated prior to performing an assay.

The present invention also provides plant and animal models in which a plant or an animal either does not express a functional BSU1 or PP1 or homologs thereof, or expresses a mutated phosphatase. Methods to produce transgenic plants and animals are well known in the art. Plant and animal models generated in this manner may be used to study activities of phosphatase polypeptides and modulating agents in vivo.

Methods for Dephosphotylating a Substrate

In one aspect of the present invention, a BSU1 or PP1 or functional equivalents or homologs thereof may be used for dephosphorylating a substrate, such as GSK3 or BEN2. In one embodiment, a substrate may be dephosphorylated in vitro by incubating a phosphatase polypeptide with a substrate in a suitable buffer (e.g., Tris. pH 7.5, 1 mM EDTA, I mM dithiothreitol, I mg/mL bovine serum albumin) for 10 minutes at 30° C. Any compound that can be dephosphorylated by the phosphatases described herein may. be used as a substrate. Dephosphorylated substrate may then be purified, for example. by affinity techniques and/or gel electrophoresis. The extent of substrate dephosphorylation may generally be monitored by adding radiolabelled phosphate labeled substrate to a test aliquot, and evaluating the level of substrate dephosphorylation as described herein.

Methods for Modulating Cellular Responses

The present invention also provides methods for modulating cellular response through BSU1 or PP1 or homologs thereof. Cellular responses may be modulated through changes in the phosphatase activity such as through mutation to the phosphatase amino acid sequence, or through contacting the phosphatase, directly or indirectly, with a modulating agent. Modulating agents may be used to modulate, modify or otherwise alter (e.g., increase or decrease) cellular responses such as cell proliferation, differentiation and survival, in a variety of contexts, both in vivo and in vitro. In general, to modulate (e.g., increase or decrease in a statistically significant manner) such a response, a cell is contacted with an agent that modulates BSU1 or PP1 or homologs thereof activity, under conditions and for a time sufficient to permit modulation of phosphatase activity. Agents that modulate a cellular response may function in any of a variety of ways. For example, an agent may modulate gene expression. A variety of hybridization and amplification techniques are available for evaluating patterns of gene expression. Further, an agent may effect apoptosis or necrosis of the cell, and/or may modulate the functioning of the cell cycle within the cell.

Treated cells may display standard characteristics of cells having altered proliferation, differentiation or survival properties. In addition, treated cells may display alterations in other detectable properties, such as contact inhibition of cell growth, anchorage independent growth or altered intercellular adhesion. Such properties may be readily detected using techniques well known to those skilled in the art.

Methods of Identifying Substances that Modulate BSU1/BIN2 and GSK3

The present invention further provides methods to screen for substances that modulate the activity of BSU1 or PP1 or homologs thereof. Substances that modulate the activity of BSU1 can be used as agents to modulate the growth in plants

The method of screening for substances comprises contacting a host cell comprising BSU1 and/or B1N2, homologs thereof, or functional fragments thereof, measuring the protein kinase and/or phosphatase activity of one or both of the BSU1 and BIN2,GSK3 proteins, and comparing the activity of one or both of the BSU1 and BIN2/GSK3 proteins in the host cell prior to contacting or in a control host cell that has not been contacted with the substance. A change in relative activity of one or both of the BSU1 and BIN2/GSK3 proteins indicates that the substance is effective in modulating those activities.

The present invention also provides methods for screening substances comprising contacting isolated BSU1 and/or BIN2/GSK3, homologs thereof, or functional fragments thereof and determining the protein kinase and/or phosphatase activity. The BSU1 and/or BIN2/GSK3, homologs thereof, or functional fragments thereof maybe isolated from cells. The cells may have been pre-treated, such as with an agent known to stimulate activity, for example brassinosteroids. The cells may have been transfected with a nucleic acid encoding the BSU1 and/or BIN2/GSK3, homologs thereof, or functional fragments thereof

The substances identified through the methods identified above, can be tested for their effects on the downstream genes regulated by this endogenous signaling pathway. For example. the substances may be tested for their ability to affect growth in plants through their effect on the signaling pathway. Further, the substances may be tested in mammalian systems for their ability to affect GSK3 activity. BSU1or PP1 or functional fragments thereof may be utilized with GSK3 to identify substances that affect GSK3.

The substance(s) identified above can be synthesized by any chemical or biological method. The substance(s) identified above can be prepared in a formulation containing one or more known physiologically acceptable diluents and/or carriers. The substance can also be used or administered to a plant or mammalian subject in need of treatment.

### EXAMPLES

Methods and Materials.

The bril-5 mutant is in WS ecotype background. and all other Arabidopsis thaliana plants are in Columbia ecotype background. The det2, BIN2-myc, bin2-1-myc, AtSK12-myc and BSU1-YFP plants for Western blotting or in vitro kinase and phosphatase assays were sterilized with bleach and grown in agar plate containing half strength (x 0.5) Murashige-Skoog (MS) medium under continuous light for 10 days. Tobacco (Nicotiana benthatniana) plants were grown in greenhouse under 16 h light/8 h dark cycles. All fusion proteins were expressed by the 35S promoter, unless indicated otherwise, in transient assays or in stable plant transformation experiments.

Phenotypic Analysis of Hypocotyls.

Sterilized Arabidopsis seeds were planted on x0.5 MS agar plate. Cold-treated agar plates were kept under white light for 6 hr s and vertically grown in the dark for 5 days. The seedlings were photocopied by digital camera.

In Vitro Kinase and Phosphatase Assays.

MBP-BZR1 and GST-BIN2 proteins were expressed and purified from E. coli and maltose or glutathione was removed from the proteins by ultrafiltration using Centricon 30 (Amicon Ultra, Millipore, Billerica, Mass.). To prepare fully phosphorylated BZR1 proteins, MBP-BZR1 protein was incubated with GST BIN2 as 1 to 1 ratio in the kinase buffer containing 100 μM ATP at 30° C. overnight. The protein mixture was incubated with glutathione Sepharose beads to remove GSTBIN2, then with amylose beads to purify MBP-pBZR1. Partially phosphorylated 32Plabeled pBZR1 and pBZR2 were prepared by the same method but MBP-BZR1 or MBP-BZR2 was incubated with GST BIN2 at a 15 to 1 ratio for 3 hrs in the presence of 20 pCi 32P-γATP. For dephosphorylation, GST-BSU1 was incubated with fully phosphorylated MBP-pBZR1 and 32P-MBP-pBZR1 or 32 P-MBP-pBZR2 for 12 or 16 Ins,

In vitro BIN2 inhibition assays were performed by 3 hrs co-incubation of MBP-BZR1, GST BIN2, GST BSU1 and ³²P-γATP or pre-incubation of GST-BIN2 with GST-BSU1 for various time followed by adding MBP-BZR1 and 32P-γATP. The examine activities of partial BSU1, N-terminal Kelch (1-363th amino acid) and C-terminal phosphatase (364-793th amino acid) region were used. GST, GST-BSU1, GSTBSU1-Ketch and GST-BSU1-phosphatase were pre-incubated with GST-BIN2 for 1 hr, and further incubated with MBP-BZR1 and ³²P-γATP for 3 hrs.

To test activities of BSU1-YFP, anti-GFP antibody-Protein A beads were used to immunoprecipitate BSU1-YFP from extracts of BSU1-YFP transgenic plants, and non-transgenic wild type plants were used as control. The beads were incubated with GST-BIN2 or GST bin2-1 for 1 hr, and then the beads were removed. The BSU1-treated GST-BIN2 or GST-bin2-1 was further incubated with MBP-BZR1 and ³²P-γATP for 3 hrs.

In vitro phosphatase assay using phospho-myelin basic protein was performed according to manufacturer's protocol (New England Biolab. Beverly, Mass.). To examine tyrosine phosphatase activity of BSU 1, 20 mM p-nitrophenyl phosphate was incubated with MBP-BSD) in 50 uL of reaction buffer (50 mM Tris. pH 7.2, 20 mM NaCl, 5 mM DTT, 10 RIM MgCl2). The reaction was quenched by the addition of 100 uL of 0.5 M NaOH after incubation at 30° C. for 1 hr. p-Nitrophenol production was determined by measuring A405 (extinction coefficient, e=1.78×104 M-tCm J).

Immunoprecipitation and Co-Immunoprecipitation.

Plant materials were ground with liquid nitrogen and resuspended in IP buffer (50 mM Tris, pH 7.5, 150 mM NaCl, 5% Glycerol, 1% Triton X-100, 1 mM PMSF and 1× protease inhibitor cocktail (Sigma)). Filtered protein extracts were centrifuged at 20,000 g for 10 min and resulting supernatant was incubated with anti-GFP antibody bound Protein A beads or anti-myc agarose beads for 1 hr. Beads were washed 5 times with washing buffer (50 MM Tris, pH 7.5, 150 mM NaCl, 0.2% Triton X-100, 1 mM PMSF and 1× Protease inhibitor cocktail). The beads were resuspended with a small volume of kinase buffer (20 mM Tris, pH 7.5, 1 mM MgCl2, 100 mM NaCl and) mM DTT) and used for in vitro phosphatase assays, or immunoprecipitated proteins were eluted with buffer containing 2% SDS and analyzed by SDS-PAGE and immunoblotting.

Dephosphorylation of phospho-tyrosine 200 residue of BIN2.

GST-BIN2 or GST-bin2-1 was incubated with MBP-BSU1 or BSU1-YFP beads for 3 Ins and subjected to immunoblotting. pTyr200 residue of BIN2 was detected by anti-phospho-GSK3a/f3 (Tyr279/216) monoclonal antibody, 5G-2F (Millipore, Temecula, Calif.) and re-probed with HRP conjugated anti-GST antibody (Santa Cruz Biotechnology, Santa Cruz, Calif.). The det2 plants were treated with 0.2 μM BL after 1 hr incubation with 10 μM MG 132. AntiBIN2 serum was developed in rabbits using GST-BIN2 as an immunogen. Monoclonal anti-GSK3α/β antibody was purchased from Invitrogen (Carlsbad, Calif.).

Site-Directed Mutagenesis.

Point mutations were generated by site-directed mutagenesis PCR according to manufacturer's protocol (Stratagene, La Jolla, Calif.). The primers used for different mutagenesis were: BIN2-Y200F-For, GAAGCCAACATTTCTTTCATCT GCTCACGATT (SEQ ID NO: 8); BIN2-Y200F-Rev, AAGCCAACATTTCTrIVATCTGCTCACGATT C (SEQ ID NO: 9); BIN2-Y200A-For, GAAGCCAACATTTCTGCCATCTGCTCACGATTC (SEQ ID NO: 10); BIN2-Y200A-Rev, GAATCGTGAGCAGATGGCAGAAATGTTGGCTTC (SEQ ID NO: 11); BIN2 MI 15A-For, CTTTTCTTGAACTTGGTTGCGGAGTATGTCCCTGAGA (SEQ ID NO: 12); BIN2 M115A-Rev, TCTCAGGGACATACTCCGCAACCAAGTTCAAGAAAAG (SEQ ID NO: 13); AtSK12 E297K-For, GAACA CCAACAAGGGAAAAAATCAAATGCATGAACCC (SEQ ID NO: 14); AtSK12 E297K-Rev, GGGTTC ATGCATTTGATTTTTTCCCTTGTTGGTGTTC (SEQ ID NO: 15), BSU1 D51ON-For, CAATCAAAGT CTTCGGCAATATCCATGGACAATAC (SEQ ID NO: 16); BSU1 D51ON-Rev, GTATTGTCCATGGAT ATTGCCGAAGACTTTGATTG (SEQ ID NO: 17).

Overexpression and Knock-Out/-Down of BSU1-Related Phosphatases.

Full-length cDNAs of BSU1 and BSL1 without stop codon were amplified by PCR using gene specific primers (BSU1-For, caccATGGCTCCTGATCAATCTTATCAATAT (SEQ ID NO: 18); BSU1-Rev, TICACTTGACTCCCCTCGAGCTGGAGTAG (SEQ ID NO: 19); BSL 1-For, caccATGGGCTCGA AGCCTTGGCTACATCCA (SEQ ID NO: 20); BSL1-Rev, GATGTATGCAAGC GAGCTTCTGTCAAA ATC (SEQ ID NO: 21)) from reverse transcription of Arabidopsis mRNA and eDNA clone (RIKEN, RAFL09-11-J01), respectively. The cDNAs were cloned into pENTR/SD/D-TOPO vectors (Invitrogen) and subcloned into gateway compatible pEarleyGate 101 or pGWB17 or pGWB20 or BiFC vectors by using LR reaction kit (Invitrogen). To test phenotypic suppression of bril-116 and bin2-1 by BSU1, 35S::BSU1-YFP single plant was crossed into bril-116 and bin2-1. The phenotype of F3 double homozygous plants was analyzed. To generate the quadruple loss-of-function mutant of bsul, bsl1/BSL2,3-amiRNA, the double mutant of bsul-1 (SALK 030721) and bsl1-1 (SALK 051383)43 was transformed with an artificial microRNA construct targeting both BSL2 and BSL3 genes (BSL2,3-amiRIVA), which was designed by the Web MicroRNA Designer 2, using the oligo (TATTCATCAAAAAGGCGCGTG (SEQ ID NO: 22)) and plasmid pRS300. The DNA fragment of amiRNA was cloned into pEarleyGate 100 (pEG 100) by using the Gateway cloning kit (Invitrogen), yielding BSL2,3-amiRNA/pEG100. The binary vector constructs were introduced into Agrobacterium strain GV3101 by electroporation and transformed into Arabidopsis by using the floral dipping method.

Quantitative RT PCR.

Quantitative real-time PCR analysis of SAUR-AC1 mRNA was performed as described by Gampala et al. using gene specific primers (SAUR-AC I -for, AAGAGGATTCATGGCGGTCTATG (SEQ ID NO: 23); SAUR-AC1-rev, GTATTGTTAAGCCGCCCA TTGG (SEQ ID NO: 24)). UBC (UBC-for, CAAATCCAAAACCCTAGAAACCGAA (SEQ ID NO: 25); UBC-rev, ATCTCCCGTAGGACCTGCACTG (SEQ ID NO: 26)) was used to normalize the loading.

Yeast Two-Hybrid Assays of AtSKs.

The cDNA clones of AtSKs were obtained from ABRC (http://www.biosci.ohio-state.edu/pcmb/Facilities/abre/abrchome.htm). All AtSKs cDNAs were subcloned into gateway compatible pGADT7 vector (Clonteeh). Nine AtSKs-pGADT7 constructs and empty pGADT7 vector were transformed into the cells containing BZR1-pGBKT7. Yeast clones were grown on Synthetic Dropout (SD) or SD-Histidine containing 2.510 mM 3-amino-1, 2, 4-triazole.

In Vitro Kinase Assay of AtSK12.

GST-AtSK12 (I μg) was incubated with MBP-BZR1 (2 μg), 100 μM ATP and 32P-γATP (10 piCi) in the kinase assay buffer for 2 hrs. The reaction was terminated by addition of 2× SDS loading buffer and separated by 7.5% SDS-PAGE. Gel was stained with Coomassie brilliant blue followed by drying. The radioactivity was analyzed by Phospho-image screen using Typhoon 8600 Scanner (GE Healthcare).

Determination of in Vitro Phosphorylation Sites of BIN2 and AtSK12.

GST BIN2 or GST-AtSK12 protein (25 μg) purified from E.coli was incubated with 100 μM ATP in the kinase buffer for 16 hrs at 30° C. Autophosphorylated GST-BIN2 or GSTAtSK12 was subjected to in-solution alkylation/tryptic digestion followed by LC-MS/MS analysis according to Gampala et al.

Overlay Western blot.

To test interaction of BSU1 with BIN2 or bin2-1 in vitro, a gel blot separating GST, GST-BIN2, GSTbin2-1 was incubated with 20 μg MBP-BSU1 in 5% non-fat dry milk/PBS buffer and washed four times. The blot was then probed with a polyclonal anti-MBP antibody. In the case of BSU1 overlay to BSK1, GST-BRI1-K, GST-BAK1-K and GST-BSK1 were separated by SDS-PAGE. To prepare phosphorylated BSK1, GST-BSK1 was incubated with GST-BRI1-K and 100 μM ATP in the kinase buffer for 2 hrs before SDS-PAGE. The blot was sequentially probed with MBP-BSU1 and a monoclonal anti-MBP antibody (New England Biolab, Beverly, Mass.).

Immunoblotting of 2-DE.

Total proteins were extracted from BL-treated or untreated 35S::TAP-BIN2 plants for two-dimensional gel electrophoresis (2-DE) as described previously. The amount of BL-treated and untreated TAP-BIN2 proteins was normalized with Western blot. Equal amount of TAP-BIN2 proteins was separated by 2-DE using an immobilized pH gradient gel strip (7 cm, pH 3-10 non-linear) and 7.5% SDS-PAGE gel. The blots were probed with anti-PAP antibody (Sigma, St. Louis, Mo.).

Transient Transformation and Confocal Microscopy.

Transformation by Agrobacterium infiltration, observations of subcellular localization and BiFC signal in tobacco or Arabidopsis were performed as described previously 5. Fluorescence of YFP was visualized by using a spinning-disk confocal microscope (Leica Microsystems, Heerbruag, Germany).

Results

Inhibition of BIN2 Activity by BSU1 Phosphatase

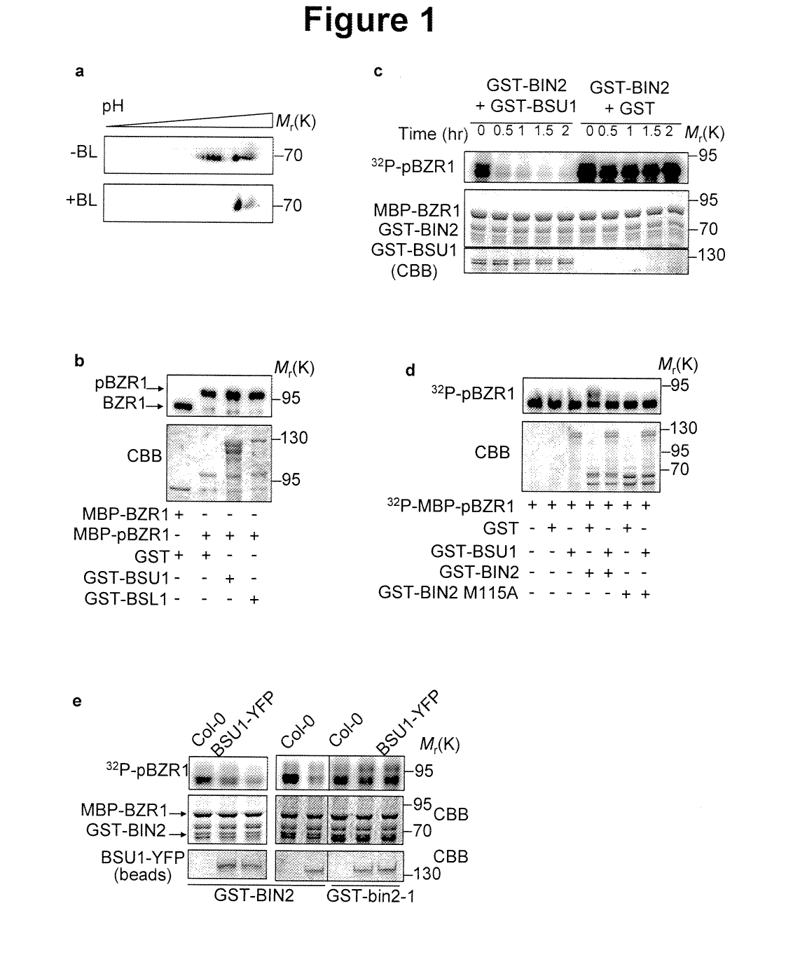

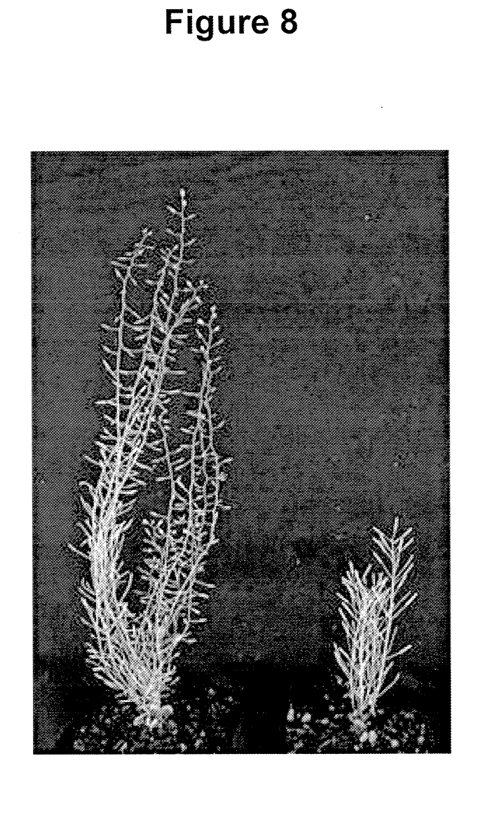

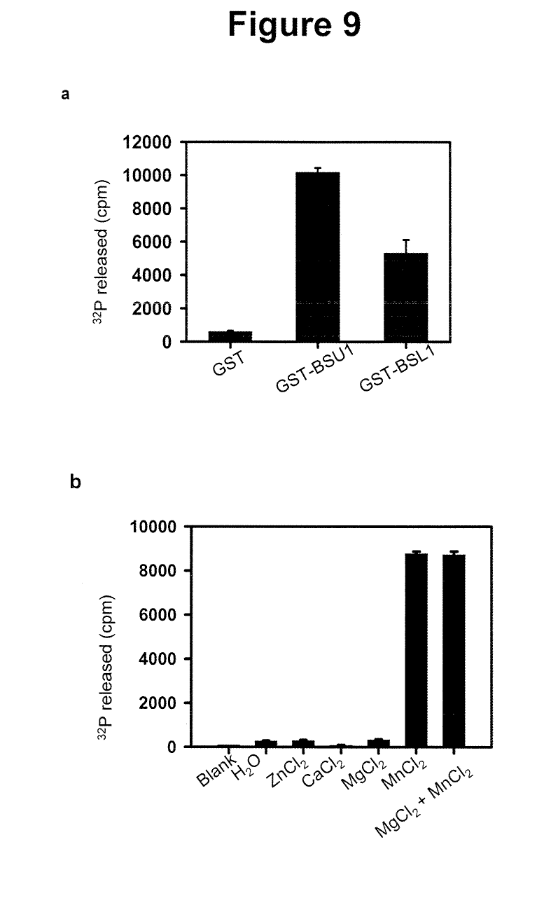

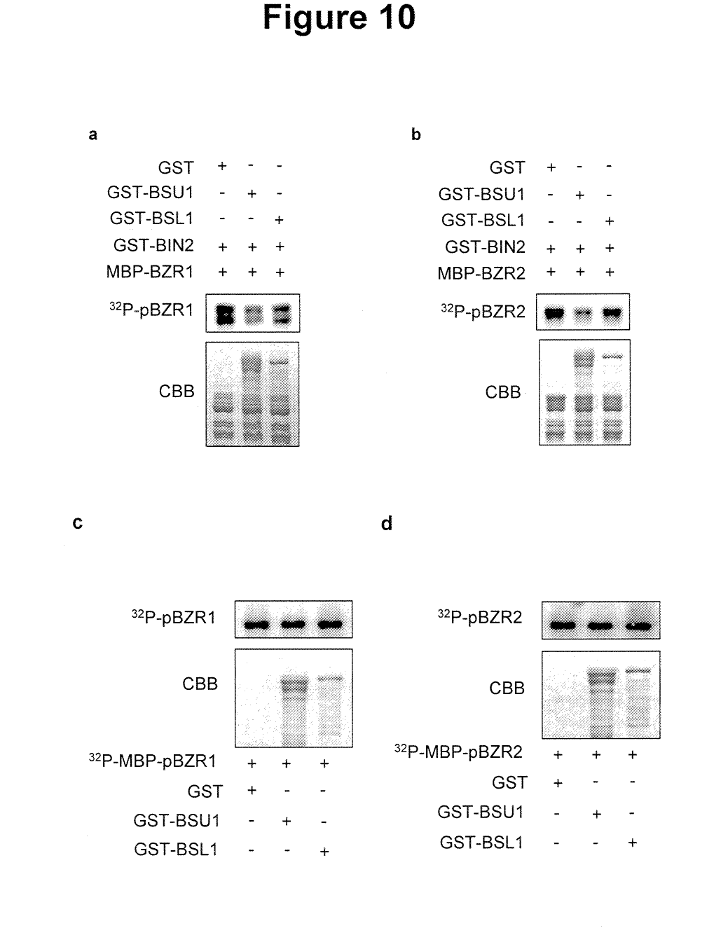

To understand how BR signaling regulates BIN2, BR-induced phosphorylation changes of BIN2 using immunoblotting of 2-dimensional gel electrophoresis was analyzed. The results showed that treatment of transgenic plants with brassinolide (BL, the most active BR) caused disappearance of the acidic forms and an increase of the basic forms of an epitope-tagged BIN2 protein (FIG. 1A), suggesting that BR induces dephosphorylation of BIN2. This result led to testing the possible role of BSU1 phosphatase in BR regulation of BIN2. Using phosphorylated myelin basic protein as substrate, both BSU1 and its closest homolog BSL1, which also promotes BR signaling in vivo (FIG. 8), showed manganese-dependent phosphatase activities (FIG. 9). BSU1 only partially reduced the phosphorylation of BZR1 when co-incubated with BIN2 and BZR1 (FIG. 10), and failed to dephosphorylate BZR1 and BZR2 when added after BIN2 and ATP were removed from the kinase reaction (FIG. 1B; FIG. 10C-D). On the other hand, BSU1 most effectively reduced the BZR1 phosphorylation when pre-incubated with BIN2 before adding BZR1 (FIG. 1C), suggesting that either BSU1 inhibits the ability of BIN2 to phosphorylate BZR1 or BIN2 is required for BSU1 to dephosphorylate BZR1. To distinguish these two possibilities, BZR1 protein was first partially phosphorylated BIN2 using radioactive ³²P-γATP followed by removal of BIN2 and ³²P-γATP, and then incubated with BIN2, BSU1 or both in the presence of non-radioactive ATP. Further phosphorylation by BIN2 using non-radioactive ATP caused a mobility shift of the pre-labeled BZR1. Addition of BSU1 did not reduce the radioactivity of ³²P-labeled BZR1, indicating no dephosphorylation of BZR1 occurred, but abolished the up shift of BZR1 band caused by BIN2 (FIG. 1D).

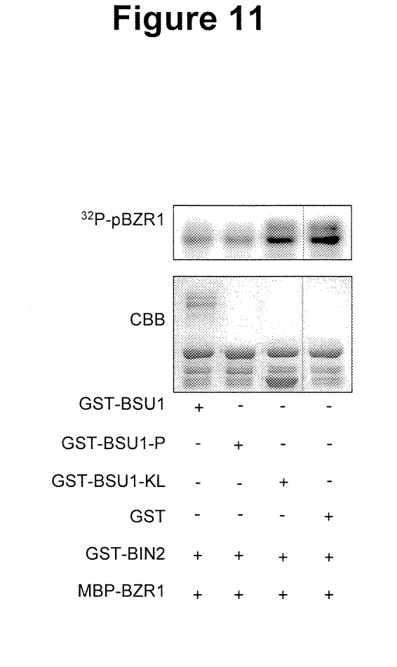

These results indicate that BSU1 inhibits BIN2 kinase activity but does not dephosphorylate pre-phosphorylated BZR1 in vitro. The phosphatase domain of BSU1 reduced BIN2 phosphorylation of BZR1 whereas the Kelch repeat domain showed no effect (FIG. 11)

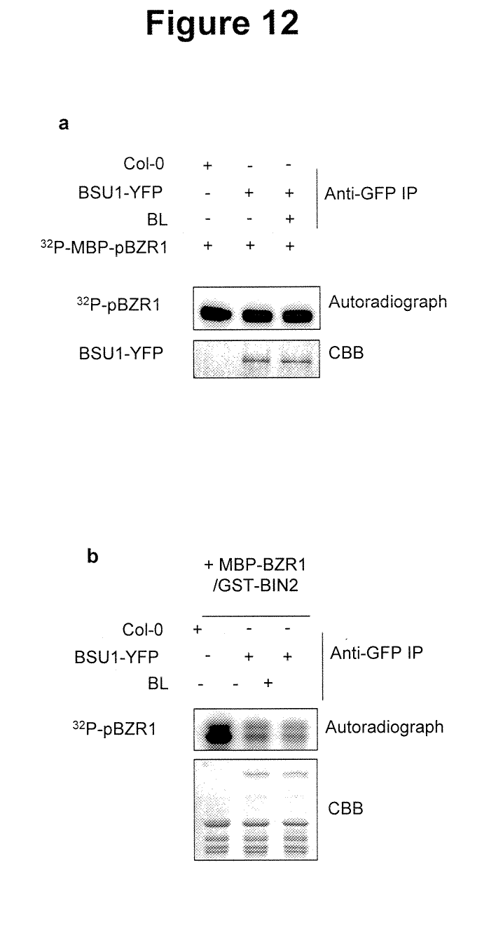

It was next examined whether BR and the bin2-1 mutation affect BSU1 inhibition of BIN2. A BSU1-YFP (yellow fluorescence protein) fusion protein was immunoprecipitated from transgenic Arabidopsis. Similar to recombinant GST-BSU1, BSU1-YFP from plants did not dephosphorylate the pre-phosphorylated BZR1 (Supplementary Information, FIG. 12A). but reduced BZR1 phosphorylation when co-incubated with BIN2 and BZR1 (FIG. 12B) or pre-incubated with BIN2 before adding to BZR1 (FIG. 1E). Moreover, BSU1-YFP from plants treated with BL showed more effective inhibition of BIN2 phosphorylation of BZR1 than that from untreated plants (FIG. 12B; FIG. 1E), suggesting that BR increases the BIN2-inhibiting activity of BSU1. The gain-of-function bin2-1 mutation causes BR-insensitive phenotypes by abolishing the inhibition of BIN2 kinase by upstream BR signaling. In contrast to wild type BIN2 kinase, the bin2-1 mutant kinase was not inhibited by BSU1-YFP (FIG. 1E), suggesting that the bin2-1 mutation causes BR-insensitive phenotypes by blocking BSU1 inhibition of BIN2.

Direct Regulation of BIN2 by BRU1 in Vivo

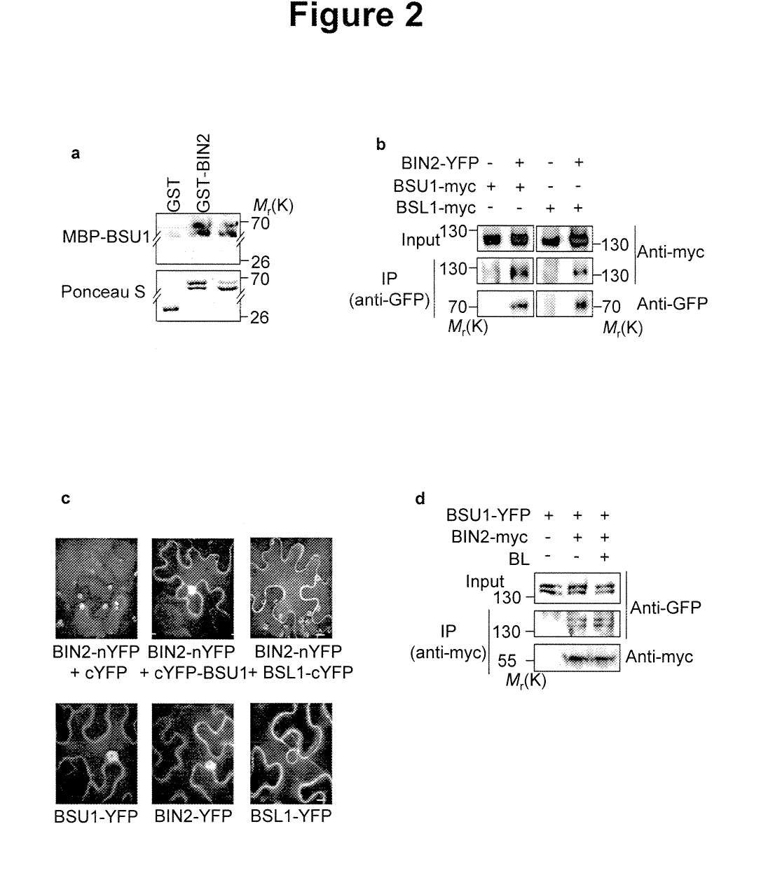

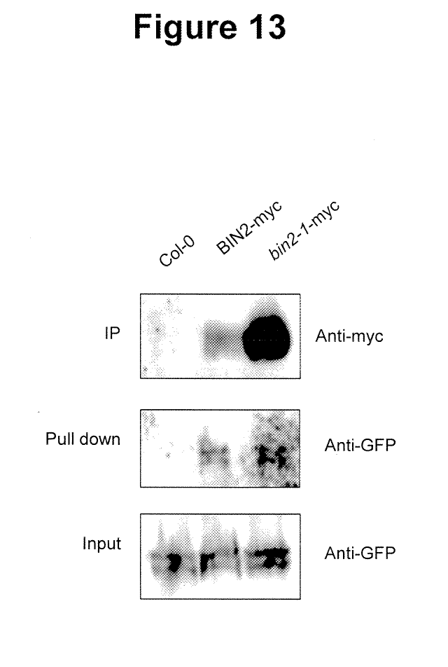

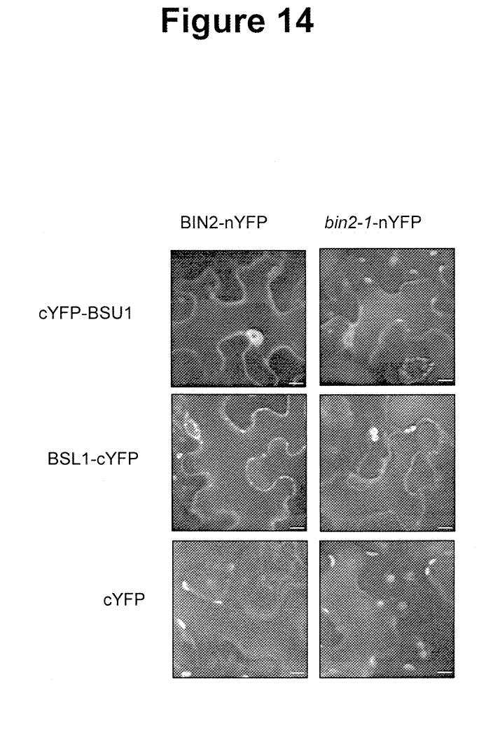

The inhibition of BIN2 by BSU1 in vitro suggests that BSU1 directly interacts with BIN2. We tested the interaction between BIN2 and BSU1 proteins in vitro and in vivo, First, GST-BIN2 was detected on a gel blot by MBP-BSU1 and anti-MBP antibody (FIG. 2A), demonstrating direct interaction between BSU1 and BIN2 in vitro. Second, the BIN2-myc protein immunoprecipitated from transgenic Arabidopsis plants pulled down BSU1-YFP from protein extracts of BSU1-YFP plants (FIG. 13), and BSU1-myc protein was co-immunoprecipitated with BIN2-YFP by anti-GFP antibodies from tobacco cells expressing both BIN2-YFP and BSU1-myc proteins (FIG. 2B). Furthermore, in vivo interaction was demonstrated by Bi-molecular Fluorescence Complementation (BiFC) assays24. Tobacco cells co-transformed with BIN2 fused to the N-terminal half (nYFP) and BSU1 fused to C-terminal half (cYFP) of YFP showed a strong fluorescence signal, whereas cells co-expressing BIN2-nYFP and non-fusion cYFP showed no fluorescence signal (FIG. 2C). Similarly, BSL1 also interacts with BIN2 in co-immunoprecipitation and BiFC assays (FIG. 2B, 2C). Importantly, co-immunoprecipitation assays showed that BR treatment increased the interaction between BSU1 and BIN2 in Arabidopsis, indicating that upstream BR signaling induces BSU1 binding to BIN2 to inhibit BIN2 activity (FIG. 2D). The BIN2-1 mutant protein also interacted with BSU1 and BSL1 in these assays (FIG. 2A; FIG. 13; FIG. 14). These results indicate that BIN2 directly interacts with BSU1 and BSL1, and the bin2-1 mutation blocks BSU1 regulation of BIN2 without abolishing their physical interaction.

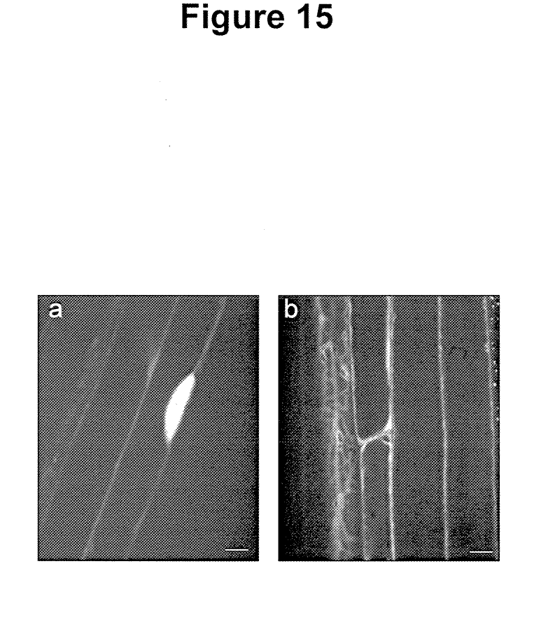

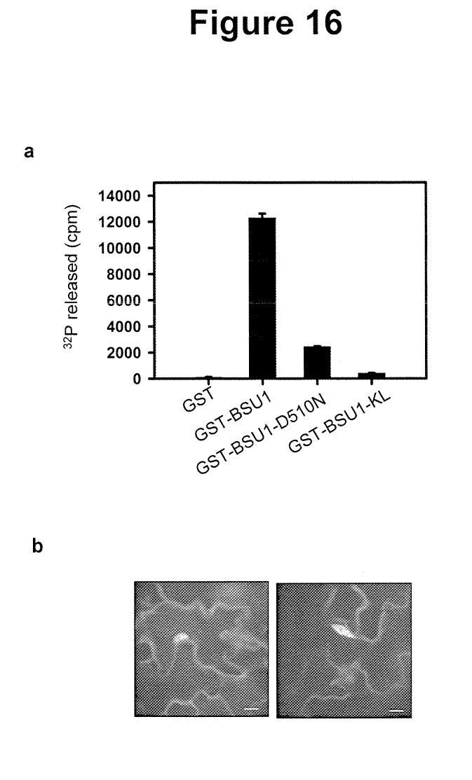

A BSU1-GFP protein was previously observed only in the nucleus. In this study, the BSU1-YFP protein expressed in Arabidopsis and tobacco leaves was detected predominantly in the nucleus but weakly in the cytoplasm (FIG. 2C; FIG. 15A). Interestingly, BSL1-YFP was excluded from the nucleus and localized exclusively in the cytoplasm and plasma membrane (FIG. 2C; FIG. 16B). In fact, BSL1 and its two other homologs have all been identified as plasma membrane proteins by recent proteomics studies, suggesting that members of the BSU family can mediate upstream BR signaling at the plasma membrane as well as act in the cytoplasm and nucleus.

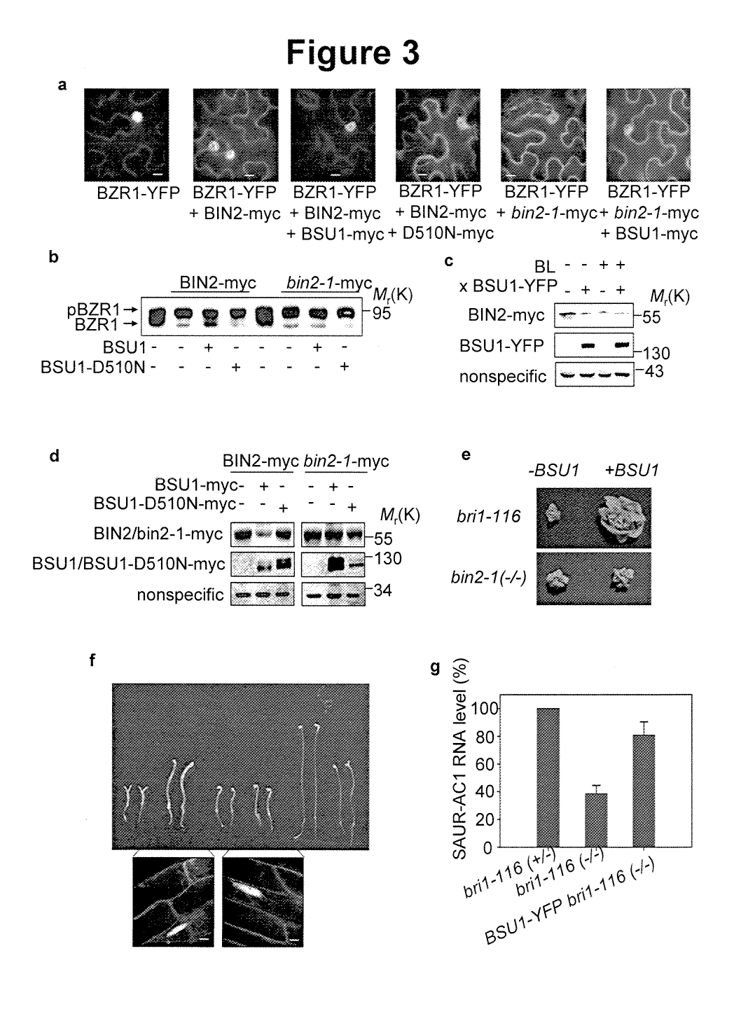

It was then further examined whether BSU1 inhibits BIN2 activity in vivo. It had previously been reported that BIN2 phosphorylation of BZR1 promotes BZR1 cytoplasmic retention by the 14-3-3 proteins while unphosphorylated BZR1 accumulates in the nucleus. It was therefore examined as to the effects of BSU1 and BIN2 on the subcellular localization and phosphorylation status of BZR1-YFP in tobacco leaves. Co-expression of BIN2 with BZR1-YFP increased phosphorylation and cytoplasmic retention of BZR1-YFP. Such an effect of BIN2 was canceled by co-expression of BSU1 (FIG. 3A, 3B), consistent with BSU1 inhibiting BIN2 phosphorylation of BZR1 (FIG. 1). The BSU1 inhibition of BIN2 depends on its phosphatase activity, because a mutant BSU1 (BSU1-D510N) with reduced phosphatase activity but normal localization (FIG. 16) failed to affect the subcellular localization and phosphorylation of BZR1-YFP in plant cells (FIG. 3A, 3B). The mutant BIN2-1 had a similar effect as wild type BIN2 on the cytoplasmic localization and phosphorylation of BZR1-YFP, however, the effect of mutant BIN2-I was not affected by co-expressing BSU1 (FIG. 3A, 3B), consistent with bin2-1 mutation abolishing BSU1 regulation of BIN2 (FIG. 1).

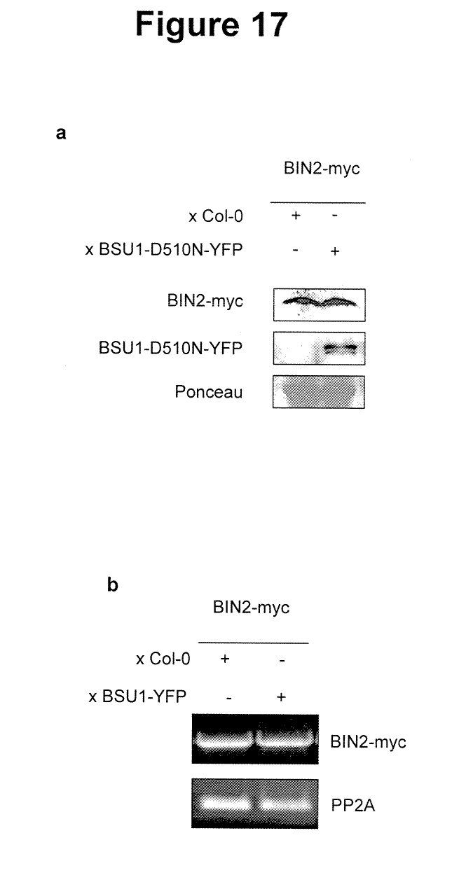

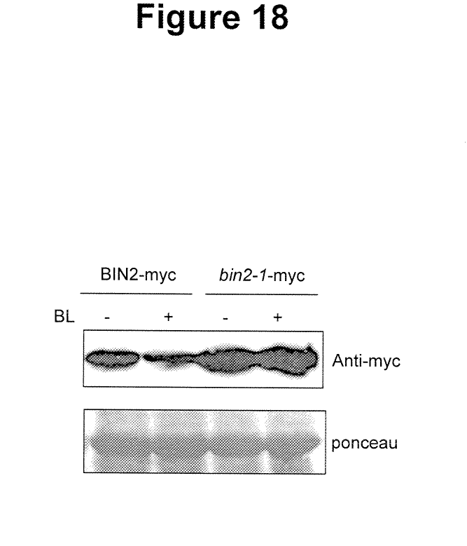

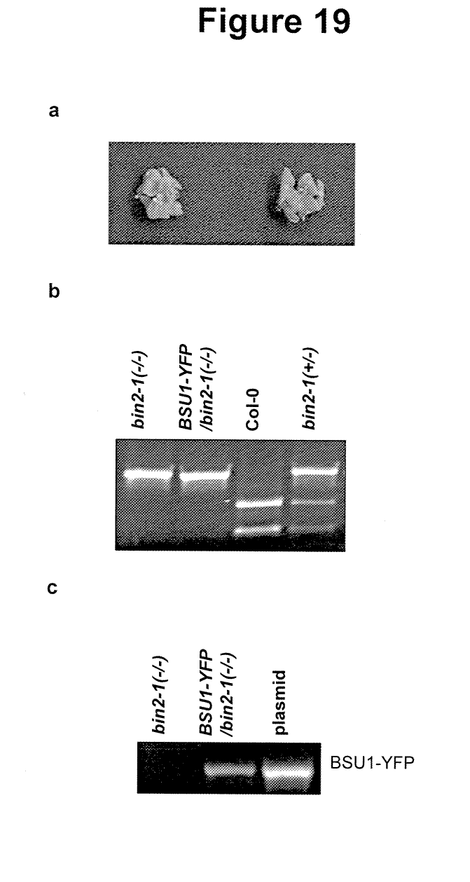

It was reported recently that BR treatment induces proteasome-mediated degradation of BIN2. To determine whether BSU1 acts upstream of BIN2 and promotes BIN2 degradation in plant cells, we crossed BSU1-YFP into BIN2-myc transgenic Arabidopsis lines. The BIN2-myc protein level was decreased by overexpression of BSU1-YFP but not by overexpression of the mutant BSU1-D51ON (FIG. 3C; FIG. 17A), while the mRNA level of BIN2-myc was unaffected (Supplementary Information, FIG. 17B). Similar to BSU1 overexpression, BR treatment also reduced the BIN2-myc protein level (FIG. 3C). BR treatment and overexpression of BSU1 reduced the accumulation of BIN2 but not bin2-1 in tobacco cells (FIG. 3d; FIG. 18). Consistent with a BSU1 function upstream of BIN2 and downstream of BRI1, overexpression of BSU1 partly suppressed the dwarf phenotype of the bril-116 null mutant but not that of homozygous bin2-1 mutant (FIG. 3E; FIG. 19). In addition, overexpression of BSU1 clearly rescued the hypocotyl elongation of bril-116 but not of the homozygous bin2-1 grown in the dark (FIG. 3F). Consistent with these developmental phenotypes, expression of the BES1-target gene, SAUR-AC119, is greatly increased in BSU1-YFP/bril-116 plants (FIG. 3G). These results demonstrate that BSU1 acts upstream of BIN2 in the BR signal transduction pathway.

Tyrosine Dephosphorylation Inhibits GSK3s

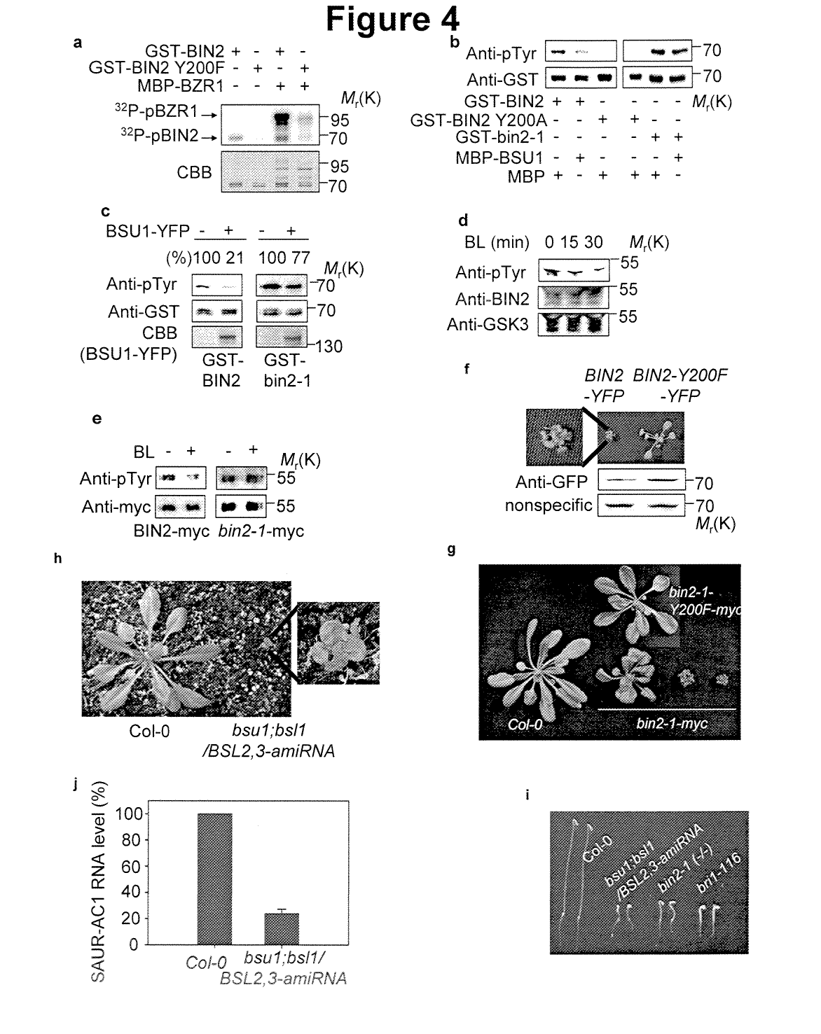

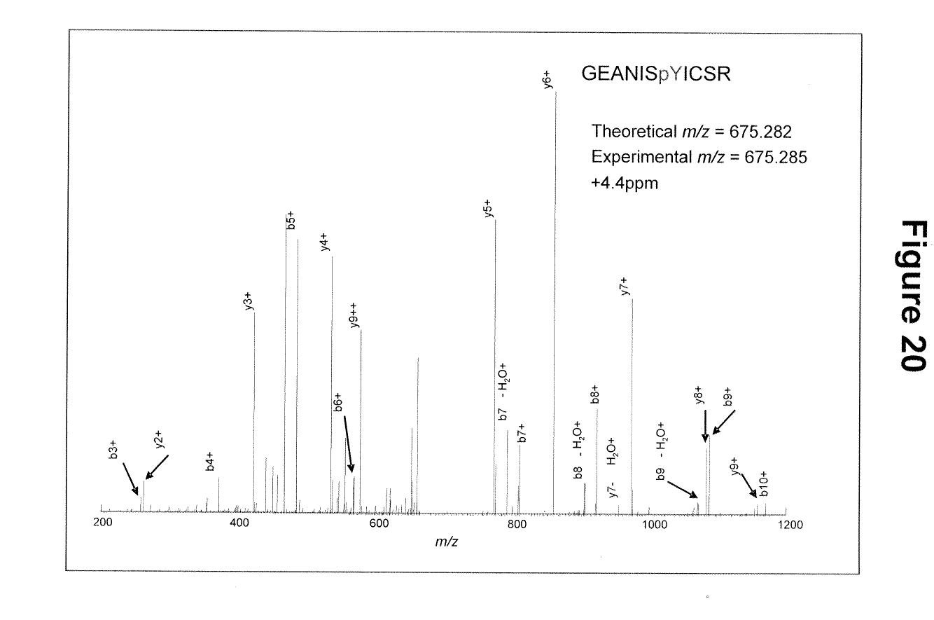

The direct interaction between BSU1 and BIN2 and the requirement of phosphatase activity of BSU1suggest that BSU1 inhibits BIN2 by dephosphorylating BIN2 during BR signaling. To understand how BSU1 inhibits BIN2 activity, first analyzed was the autophosphorylation sites of BIN2 in vitro using mass spectrometry. Phospho-tyrosine 200 (pTyr200) of BIN2 was identified as a major phosphorylation site (FIG. 20). The same residue was recently detected as an in vivo phosphorylated site of BIN2 by a phosphoproteome analysis of Arabidopsis. This Tyr residue lies within the activation loop of the catalytic domain and is highly conserved in all GSK3s of worms, flies, fungi, vertebrates, and plants. Its phosphorylation is, essential for the full GSK3 kinase activities in mammals and Dictyostelium. Likewise, phosphorylation of Tyr200 residue is required for full BIN2 activity, as mutation of Tyr200 to Phe (Y200F) in BIN2 greatly reduced its substrate phosphorylation (FIG. 4A).

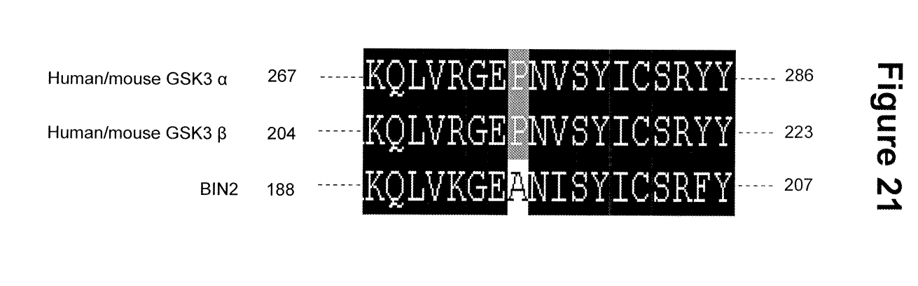

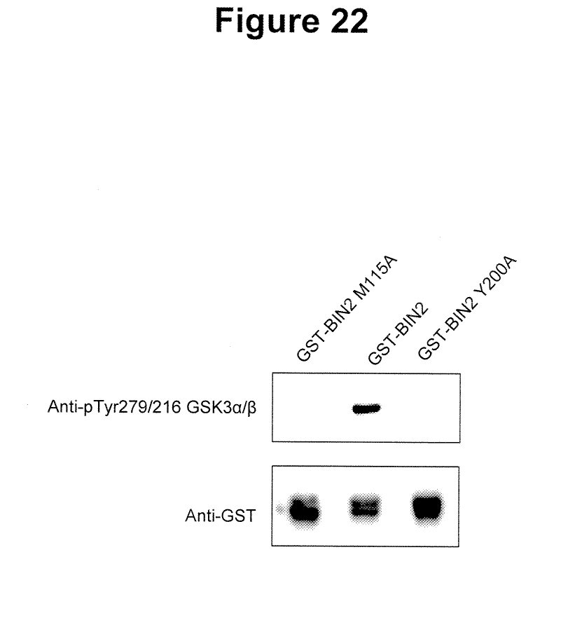

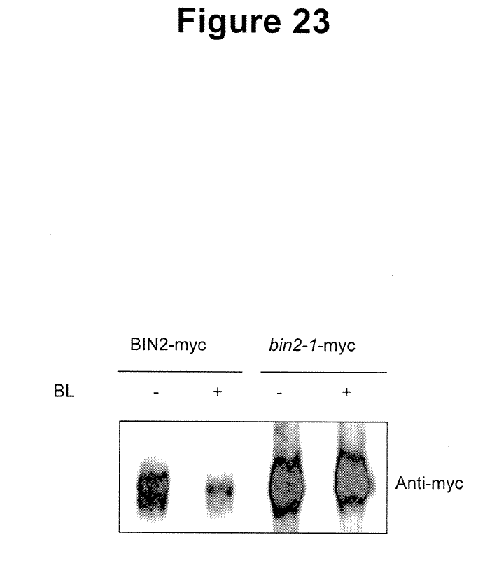

The amino acid sequence flanking the Tyr200 of BIN2 is highly conserved in mammalian GSK3s (FIG. 21), and a monoclonal antibody for phospho-Tyr216 of human GSK3p specifically detected wild type GST-BIN2 but not the GST-BIN2 containing Y200A mutation or the kinase-inactivating M115A mutation (FIG. 4B; FIG. 22), indicating that this antibody can specifically detect the phospho-Tyr200 residue of BIN2. The results also suggest that the BIN2 kinase activity is required for Tyr200 phosphorylation, similar to mammalian GSK3. Based on the signal level detected by this antibody, incubation with BSU1 from E. coli (FIG. 4B) or BSU1-YFP from plants (FIG. 4C) greatly reduced Tyr200 phosphorylation of BIN2, but had little effect on that of bin2-1. We further investigated whether BR regulates the dephosphorylation of pTyr200 of BIN2 in plants. In the presence of the proteasome inhibitor MG132, which prevents BR-induced BIN2 depletion23 (FIG. 23), BL treatment reduced the phosphorylation of Tyr200 of the wild type BIN2 (FIG. 4D) or BIN2-myc. but not that of the mutant bin2-1-myc (FIG. 4e). These results demonstrate that BR signaling inhibits BIN2 through BSU1-mediated dephosphorylation of pTyr200, and the bin2-1 mutation causes BR insensitivity by blocking this dephosphorylation. The effects of bin2-1 mutation on BSU1 regulation in vitro and in vivo strongly support a role of BSU1-mediated tyrosine dephosphorylation as the primary mechanism of BIN2 regulation essential for BR signal transduction.

To further confirm the role of Tyr200 phosphorylation for BIN2 regulation in vivo. we tested the effects of a Y200F mutation on growth and development in transgenic plants. While overexpression of wild type BIN2 or mutant bin2-1 causes BR-insensitive dwarf phenotypes in transgenic Arabidopsis plants, overexpression of BIN2 or bin2-1 containing the Y200F mutation did not (FIG. 4F, 4G), indicating that Tyr200 phosphorylation is essential for BIN2 to inhibit BR-dependent plant growth and that dephosphorylation of pTyr200 is sufficient to inactivate BIN2. In contrast to Y200F mutation but similar to the bin2-1 mutation, quadruple loss-of-function of BSU1and its three homologs by T-DNA insertion and artificial microRNA caused severe dwarf phenotypes (FIG. 4H, I). Furthermore, the expression level of the BEST-target gene SAUR-AC1 is greatly reduced in the quadruple mutant (FIG. 4J). Taken together, these results demonstrate that dephosphorylation of BIN2 by the BSU1-related phosphatases is an essential step of BR signal transduction required for BR regulation of plant growth.

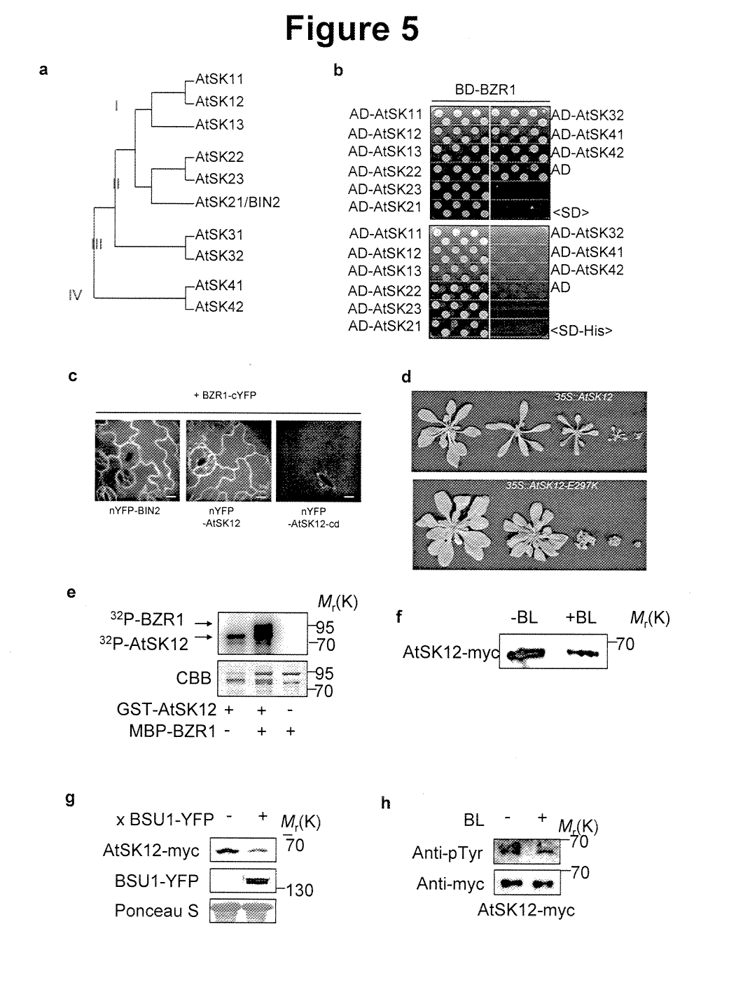

The Arabidopsis genome encodes 10 GSK3/Shaggy-like kinases (AtSKs), which are classified into four subgroups (FIG. 5A). Recently, it was reported that a triple knockout mutant plant for group II including BIN2 show increased cell elongation but still accumulates phosphorylated BES I and responds to BL, indicating that other GSK3-like kinases also act in BR signaling. To determine how many AtSKs are involved in BR signaling, first performed was an interaction study between BZR1 and nine AtSKs representing four subgroups. Interestingly, all six AtSKs belonging to subgroup I and II showed interaction with BZR1 in yeast two-hybrid assay (FIG. 5B). The function of AtSK12 as a representative of subgroup I AtSKs in BR signaling was further examined.

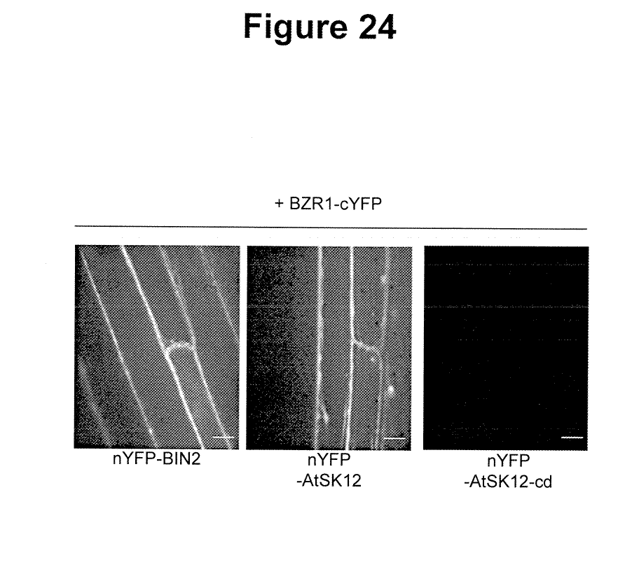

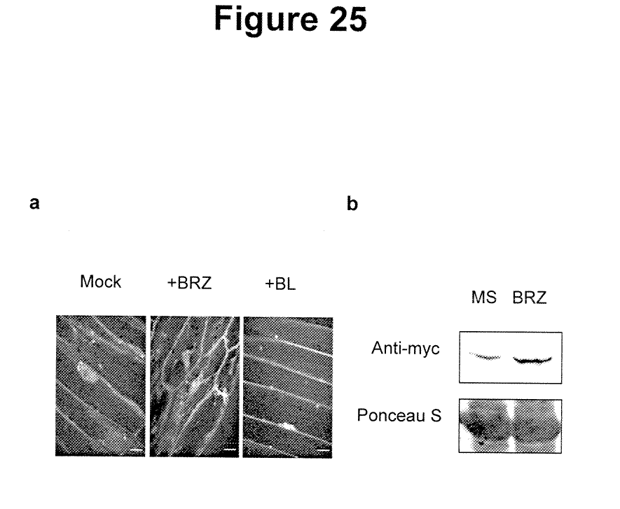

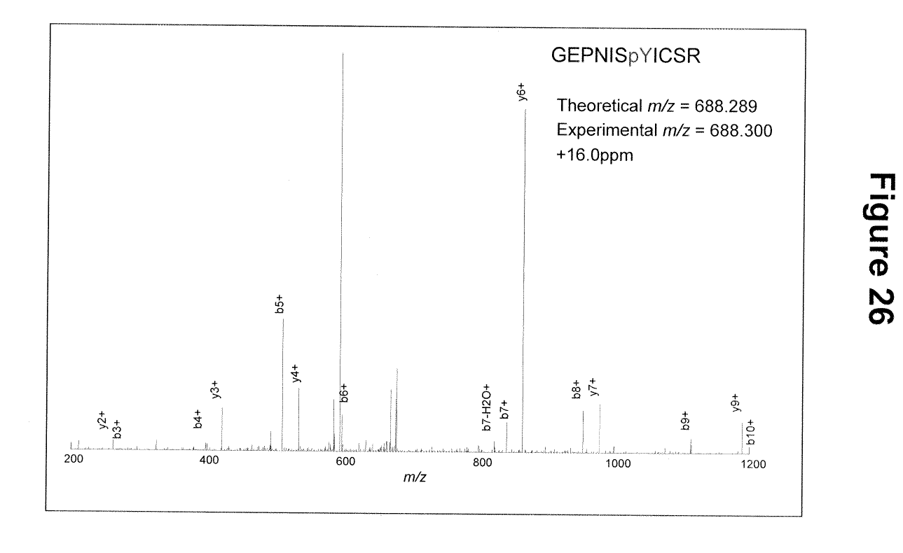

Consistent with interaction in yeast, BiFC assay showed that AtSK12 interacts with BZR1 as does BIN2 in Arabidopsis, and deletion of the C-terminal 29 amino acids of AtSK12 abolished the interaction with BZR1 (FIG. 5C; FIG. 24). Transgenic plants overexpressing AtSK12 displayed similar dwarf phenotypes as those overexpressing BIN2 (FIG. 5D). Moreover, overexpression of AtSK12-E297K corresponding to the bin2-1 gain-of-function mutation caused more severe phenotype than overexpression of wild type AtSK12 (FIG. 5D). In vitro kinase assay using GST-AtSK12 and MBP-BZR1 showed that AtSK12 strongly phosphorylates BZR1 in vitro (FIG. 5E), suggesting that BZR1 is a substrate of AtSK12. Similar to BIN2 (FIG. 2C), AtSK12 protein is localized in both cytoplasm and nucleus independent of BR (FIG. 25A), stabilized by the BR biosynthetic inhibitor brassinazole (BRZ) (FIG. 25B), and destabilized by BL (FIG. 5F) and by overexpression of BSU1-YFP (FIG. 5G), indicating that AtSK12 is also regulated by BR and BSU1. Mass spectrometry analysis indicated that Tyr233 of AtSK12 (corresponding to Tyr200 of BIN2) was also phosphorylated (FIG. 26). In the presence of MG132, BL treatment greatly reduced phosphorylation of AtSK12 Tyr233, indicating that regulation of AtSK12 by BR signaling involves Tyr233 dephosphorylation (FIG. 5H). These results suggest that BSU1-mediated tyrosine dephosphorylation is a common mechanism shared by at least two of six GSK3-like kinases that are likely involved in BR signaling.

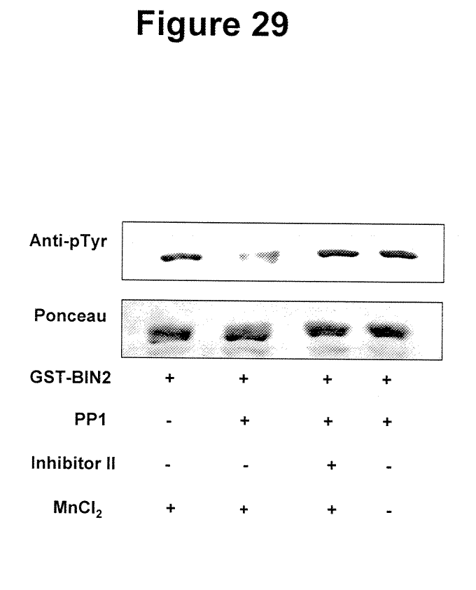

It was next examined whether the mammalian homolog to BSU1, PP1, would dephosphorylate BIN2. A GST-tagged BIN2 was isolated from cells and incubated with PP1 purified from E. coli cells expressing the phosphatase. The presence of PP1 increased dephosphorylation of BIN2 tyrosine200 (FIG. 29). The PP1 inhibitior, PP2 (protein phosphatase inhibitor 2), inhibited the enzymatic activity of the PP1 phosphatase on BIN2 (FIG. 29). Similarly, the phosphatase inhibitor, manganese chloride also inhibited the enxymatic activity of PP1 on BIN2 (FIG. 29).

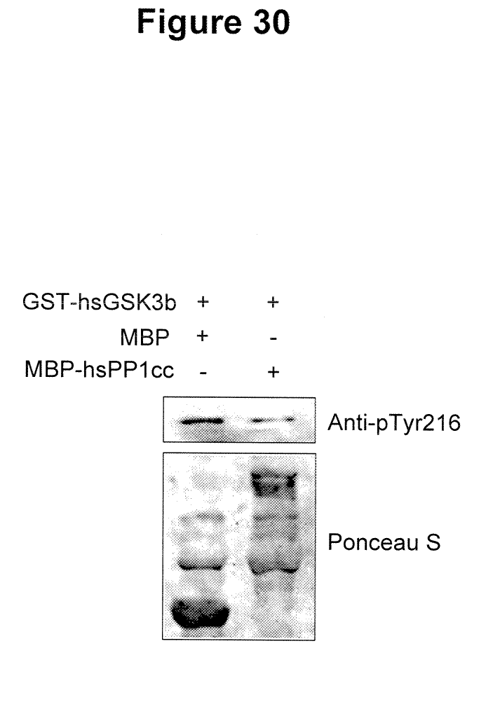

To determine whether PP1 regulates GSK3 kinase activity in mammals, it was further examined whether human protein phosphatase I gamma (PP1γ) dephosphorylates tyrosine 216 of human GSK3 beta in vitro. A GST-tagged human GSK3-beta (GST-hsGSK3-beta) was isolated from E. coli and incubated with human PP1-gamma purified from E. coli cells expressing the phosphatase. The presence of PP1 increased dephosphorylation of GSK3-beta tyrosine216 (FIG. 30).

BR11-phosphorylation Promotes BSK1 Binding to BSU1

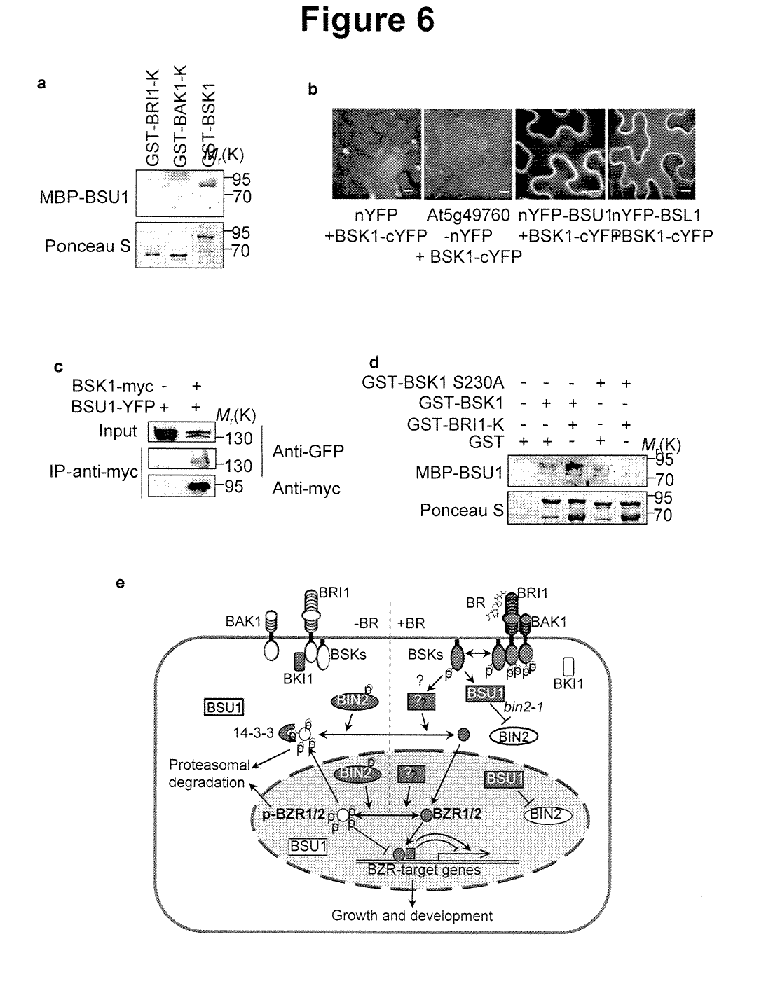

The function of BSU1 upstream of BIN2 suggests that it might be directly regulated by upstream components on the plasma membrane. Direct interaction of BSU1 with BRII, BAK1 and BSK1 was tested in an in vitro overlay assay. As shown in FIG. 6A, the MBP-BSU1 protein interacted with BSK1 but not with BRI1 or BAK1, which is consistent with BSK1 being downstream of BRI1 in the signaling pathway. BiFC assays showed that BSK1 interacts with both BSU1 and BSL1 in vivo (FIG. 6B). The in vivo interaction was further confirmed by co-immunoprecipitation assays using transgenic Arabidopsis plants expressing both BSK1-myc and BSU1-YFP proteins (FIG. 6C). It has been previously shown that BRI1 phosphorylates BSK1 at Ser230. It was thus tested whether BRI1 phosphorylation of Ser230 affects BSK1 binding to BSU1. Indeed, phosphorylation of BSK1 BRI1 increased the binding while mutation of S230A abolished the binding of BSK1 to BSU1 (FIG. 6d), indicating that BRI1 phosphorylation of BSK1 at Ser230 increases its interaction with BSU1. These results demonstrate that BRI1 phosphorylation of BSK1 Ser230 promotes BSK1 binding to BSU1. Such interaction with BSK1 is likely to mediate BR activation of BSU1 in vivo, although an effect of BSK1 on BSU1 activity in vitro was not detected (data not shown). Together these results bridge the last major gaps and elucidate a complete BR signal transduction cascade from cell-surface receptor kinases to nuclear transcription factor (FIG. 6E).

Discussion

Signal transduction through cell surface receptor kinases is a fundamental mechanism for cellular regulation in living organisms. BRI1 is a member of the large family of leucine-rich-repeat receptor-like kinases (LRR-RLK), with over 220 members in Arabidopsis and 400 in rice. Only a handful of these RLKs have been studied and a complete RLK-signaling pathway that involves multiple steps of sequential mediated signaling pathway has not been elucidated in plants. This work illustrates a complete signal transduction pathway that links BR-BRI I binding at the cell surface with activation of BZR transcription factors in the nucleus (FIG. 7B; FIG. 6D). In the absence of BR, BZR1 and BZR2 are inhibited by BIN2-catalyzed phosphorylation and consequent binding by the 14-3-3 proteins 4. BR binding to the extracellular domain of BRI1 activates BRI1 kinase through ligand-induced association and trans-phosphorylation with its co-receptor kinase BAK1. BRI1 then phosphorylates the BSK1 kinase at Ser230, and this phosphorylation promotes BSK1 interaction with BSU1. BSK1 is likely to mediate BR activation of BSU1 in vivo, although BSK1 did not affect BSU1 activity in vitro (data not shown). Upon activation by BR signaling, BSU1 dephosphorylates BIN2 at the pTyr200 residue to inhibit its kinase activity, allowing accumulation of unphosphorylated BZR1 and BZR2 in the nucleus, where they bind to promoters and regulate BR responsive gene expression and plant growth (FIG. 6D: 7B). This study has therefore elucidated a complete BR phosphorylation/dephosphorylation cascacde that transduce the signal from BRI1/BAK1 receptor kinase complex to BSK1. BSU1, BIN2. and BZR1/BZR2. This fully connected BR signaling pathway provides a paradigm for understanding both RLK-mediated signal transduction and steroid signaling through cell surface receptors.

Interestingly, each component of the BR signaling pathway is encoded by a small gene family with three to six members that appear to have similar biochemical functions. BRI1 is the only component of the BR signaling pathway that was identified by recessive mutations, indicating its essential role in BR regulation of plant growth. However, two BRI1 homologs, BRL1 and BRL3 can genetically complement the bril mutant when expressed from the BRI1 promoter and they bind BR with similar affinity as BRI111. It is believed that BRL 1 and BRL3 mediate BR signaling in a tissue specific manner. All the other components of the BR signaling pathway were identified either by gain-of-function mutations or by proteomic/biochemical approaches. Genetic analyses of loss-of-function alleles of these components indicated genetic redundancy among the members of each gene family. Single knockout of BIN2. BZR1, BES1, BSU1, and BSK1 caused no obvious phenotype or very subtle growth phenotypes. Triple knockout of BIN2 and its two close homologs (Group II GSKs) showed enhanced cell elongation, but still contained significant amount of phosphorylated BEST, suggesting additional members of the GSK3 family are involved in BES1 phosphorylation. Consistent with these previous studies, it was found that six members of the Group I and II GSK3s can interact with BZR1 in yeast. Overexpression and biochemical studies of a group I member, AtSK12, provide strong evidence that Group I GSK3s are also involved in BR signaling (FIG. 5). Loss-of-function mutations of additional family members will likely be required to elucidate the functional relationship among members of GSK3s in BR regulation of plant growth. Similarly, knockdown expression of two BSU1 homologs (BSL2, and BSL3) by RNAi caused a weak dwarf phenotype. In contrast, knockdown expression of BSL2 and BSL3 in the bsu1/bsl1 double mutant background caused severe dwarf phenotypes, indicating that members of the BSU1 family play redundant or overlapping roles in BR signal transduction. As such, it appears that each step of BR signal transduction can be carried out by one of several members of the gene family, although only the founding member of each family is presented in the conceptual model of BR signal transduction (FIG. 6E).

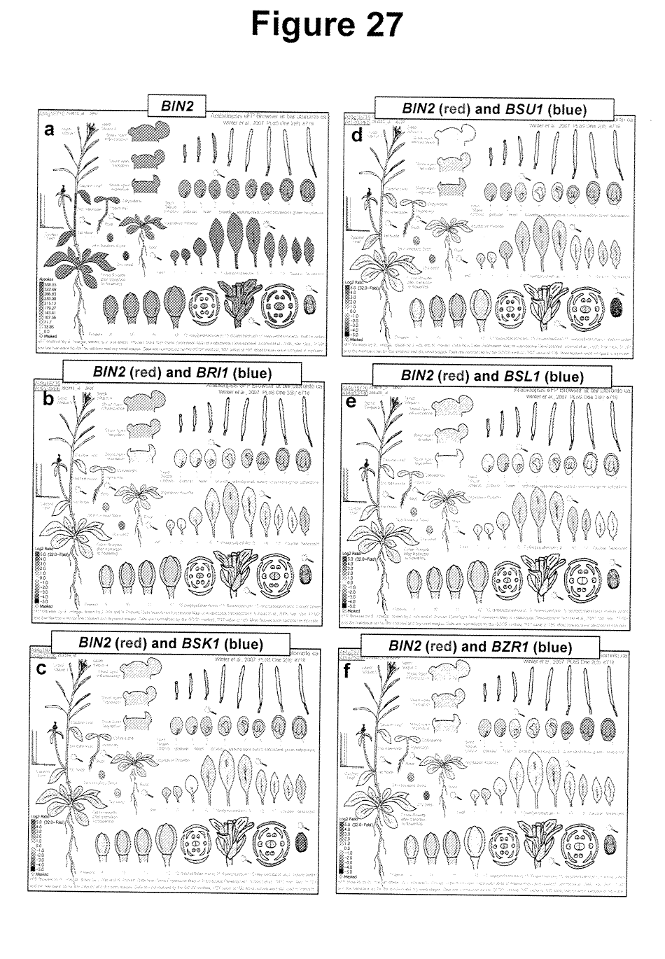

The presence of multiple genes encoding same signaling function can potentially be beneficial in several ways. First, different family members might provide activity in different subcellular compartments, as suggested by the complementary localization patterns of BSU1 in the nucleus and BSL1 in the cytoplasm and at the plasma membrane. Because BIN2 is localized in both nucleus and cytoplasm, it is likely that BSU1 and BSL1 together provide regulation of BIN2 at the plasma membrane, in the cytoplasm and nucleus. Although these data indicate that BSU1 and BSL1 both regulate BIN2 in a similar manner, the possibility that there are qualitative or quantitative differences in the signaling activity or specificity of different family members cannot be excluded. Second, different promoters of family members can provide tissue specificity and flexibility for transcriptional regulation of BR signaling components by developmental programs and environmental cues. The presence of gene families also raises an important question about the heterogeneity of the BR signaling pathway in different tissues and cell types. Different gene family members can be expressed in different cells to assemble BR signaling pathways of different composition. Although the evidence available no far supports the notion that these family members play similar biochemical function and thus there is a general model of BR signal transduction (FIG. 6E), it is possible that the heterogeneity in pathway composition provides diversity of functional specificity. Future genetic analysis of mutants defective in various combinations of family members can provide some clues about the functional specificity or redundancy. However, such genetic analysis can also be complicated by competition and replacement between family members; a protein might gain new function when a competing homolog is knocked out. The gene expression patterns in wild type plants, on the other hand, provide a good estimate of which family members are likely to function together in natural condition. Based on available microarray data, BSU1 shows a very similar expression pattern to BRI1, BSK1, BIN2, and BZR1, except its higher expression level in pollen (FIG. 27). Such similar ubiquitous expression patterns are consistent with the genetic evidence for their functions as major players in the BR regulation of plant growth and development.

This study reveals BSU1-mediated pTyr200 dephosphorylation as the primary mechanism for regulating plant GSK3s in the BR signaling pathway. The importance of this mechanism for BR signal transduction and plant growth regulation is supported by the strong opposite effects on plant growth of the mutations that impair dephosphorylation (bin2-1 and quadruple bsu1bs11/BSL2,3-amiRNA mutations) and phosphorylation (bin2-Y200F) of Tyr200. This tyrosine residue is absolutely conserved in all GSK3s identified so far. In Dictyostelium, dephosphorylation of the conserved tyrosine (Tyr214) of GSK3 is a key mechanism for cell surface receptor-mediated cAMP regulation of cell differentiation, but the phosphatase for this regulation has not been identified. Interestingly, the mechanism of BIN2 inactivation by BR is distinct from those of GSK3 inactivation by the Writ signaling pathway in mammals, despite the similarity between BIN2 and mammalian GSK3I3 in their structure and mode of action on substrates. The catalytic domain of BIN2 shares 70% sequence identity to that of human GSK3β, which plays key roles in a range of cellular and disease processes. Furthermore, BIN2 regulation of BZR1/BZR2 resembles GSK3β regulation of β-catenin in the Writ signaling pathway, in which the phosphorylation by GSK3β of β-catenin leads to its degradation in the absence of Wnt and Wnt signaling leads to nuclear accumulation of dephosphorylated β-catenin. By, contrast to the BR pathway, Wnt signaling inhibits GSK3β by disrupting a protein complex containing GSK3β, axin, and β-catenin. On the other hand, phosphorylation of Tyr216 of GSK3β (Tyr279 in GSK3α), corresponding to Tyr200 of BIN2. is required for kinase activity, and change of Tyr216 phosphorylation level has been observed during neuron cell death in Alzheimer's disease and upon perturbation of the Writ signaling pathway. However, a key function of tyrosine dephosphorylation has not been demonstrated in these processes, and it remains unclear whether tyrosine dephosphorylation has been replaced by other mechanisms or still used in specific pathways that are not fully understood in mammals.

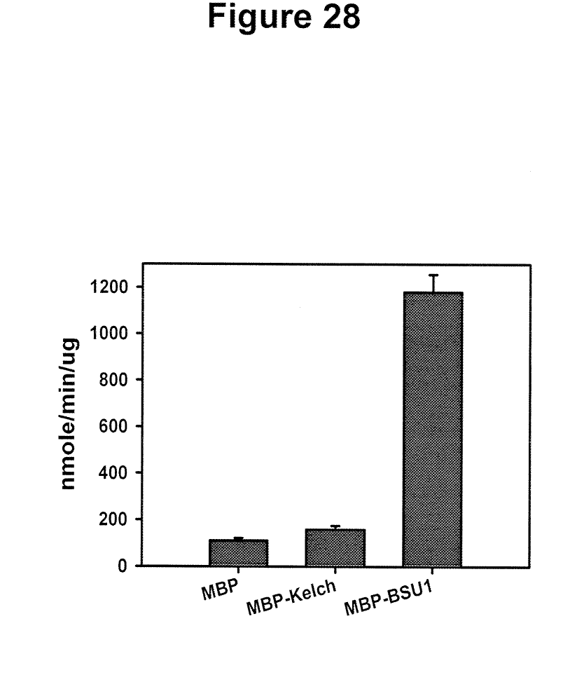

BSU1 represents the first phosphatase that mediates dephosphorylation of this conserved tyrosine residue of GSK3s. BSU1 contains an N-terminal Kelch-repeat domain and a C-terminal phosphatase domain. Although BSU1 phosphatase domain was classified into Ser/Thr phosphatase. these results indicate that BSU1 is a dual specificity protein phosphatase that dephosphorylates both phospho-Ser/Thr (FIG. 9) and phospho-Tyr (FIG. 28) residues. In vitro phosphatase assays using BSU1 expressed in either E. coli or plants indicate that BSU1 directly dephosphorylates Tyr200 of BIN2, though there remains the possibility that BSU1 also dephosphorylates Ser/Thr residues on GSK3s. The phosphatase domain of BSU1 shares about 45% sequence identity with mammalian protein phosphatase-1 (PP1). Interestingly, PP1 expressed in E. coli exhibits both Tyr and Ser/Thr phosphatase activity, although native PP1 expressed in mammalian cells is inactive on phospho-Tyr due to inhibition by inhibitor-2, which is a substrate of GSK3. It will be interesting to see if BSU1-related phosphatases mediate tyrosine dephosphorylation of GSK3s in mammals and other species. These studies of the BR signaling pathway not only provide insight into plant growth regulation by steroid hormones. but also shed new light on the mechanisms of GSK3 regulation.

Human Protein Phosphatase 1 Gamma (PP1γ) Dephosphorylates BIN2 and Tyrosine 216 of Human GSK3 Beta in Vitro

A GST-tagged BIN2 was isolated from cells and incubated with PP1 purified from E. coli cells expressing the phosphatase. The presence of PP1 increased dephosphorylation of BIN2 tyrosine200 (FIG. 29). The PP1 inhibitior, PP2 (protein phosphatase inhibitor 2), inhibited the enzymatic activity of the PP1 phosphatase on BIN2 (FIG. 29). Similarly, the phosphatase inhibitor, manganese chloride also inhibited the enxymatic activity of PP1 on BIN2 (FIG. 29).

It was next examined whether PP1 would dephosphorylate GSK. 2 μg of MBP or MBP-hsPPP1cc was incubated with 1 μg of GST-hsGSK3β in phosphatase assay buffer (50 mM HEPES pH 7.5, 100 mM NaCl, 2 mM DTT, 0.01% Brij 35 and 1 mM MnCl2) for 3 hrs at 30° C. After incubation. proteins were separated by 7.5% SDS-PAGE gel followed by blotting onto nitrocellulose membrane. The blot was probed with anti-phospho-tyrosine 216 of GSK3β antibody to test phosphorylation status of hsGSK3β. FIG. 30 shows that human protein phosphatase 1 gamma (PP1γ) dephosphorylates tyrosine 216 of human GSK3 beta in vitro.

### REFERENCES

1. Clouse. S. D. & Sasse, J. M. BRASSINOSTEROIDS: Essential Regulators of Plant Growth and Development. Annu Rev Plant Physiol Plant Mel Bio/49, 427-451 (1998).

2. Belkhadir, Y., Wang, X. & Chory, J. Arabidopsis brassinosteroid signaling pathway. Sci STKE2006, cm5 (2006).

3. Vert, G., Nemhauser, J. L., Geidner, N., Hong, F. & Chory, J. Molecular mechanisms of steroid hormone signaling in plants. Annu Rev Cell DevB/o/21, 177-201 (2005).

4. Gendron. J. M. & Wang, Z. Y. Multiple mechanisms modulate brassinosteroid signaling. Curr Opin Plant Siol 10, 436-441 (2007).

5. Li. J. & Chory. J. A putative leucine-rich repeat receptor kinase involved in brassinosteroid signal transduction. Ce/190, 929-938 (1997).

6. Wang, Z. Y., Seto, H., Fujioka, S., Yoshida, S. & Chory, J. BRI1 is acritical component of a plasma-membrane receptor for plant steroids. Nature410, 380-383 (2001).

7. Li, J. et al. BAK1, an Arabidopsis LRR receptor-like protein kinase, interacts with BRI1 and modulates brassinosteroid signaling. Celt 110, 213-222 (2002).

8. Nam, K. H. & Li, J. BRI1/BAK1, a receptor kinase pair mediating brassinosteroid signaling. Ce//110. 203-212 (2002).

9. Wang, X. & Chory, J. Brassinosteroids regulate dissociation of BKI1, a negative regulator of BRI1 signaling, from the plasma membrane. Science313, 1118-1122 (2006).

10. Tang, W. et a/. BSKs mediate signal transduction from the receptor kinase BRI1 in Arabidopsis. Science 321, 557-560 (2008).

11. Kinoshita. T. et al. Binding of brassinosteroids to the extracellular domain of plant receptor kinase BRI1. Nature433, 167-171 (2005).

12. Wang, X. et a/. Sequential transphosphorylation of the BRI1/BAK1 receptor kinase complex impacts early events in brassinosteroid signaling. Dev Celt 15. 220-235 (2008).

13. Li, J. & Nam, K. H. Regulation of brassinosteroid signaling b a GSK3/SHAGGY-like kinase. Science295, 1299-1301 (2002).

14. Mora-Garcia, S. et a/ Nuclear protein phosphatases with Kelch-repeat domains modulate the response to brassinosteroids in Arabidopsis. Genes Dev 18, 448-460 (2004).

15. Gampala. S. S. et al. An essential role for 14-3-3 proteins in brassinosteroid signal transduction in Arabidopsis. DevCell 13. 177-189 (2007).

16. Wang, Z. Y. et a/ Nuclear-localized BZR1 mediates brassinosteroid-induced growth and feedback suppression of brassinosteroid biosynthesis. DevCei/2, 505-513 (2002).

17. Yin, Y. et a/ A crucial role for the putative Arabidopsis topoisomerase VI in plant growth and development. Proc Nal/Acad Sci USA99, 10191-10196 (2002).

18. He, J.-X. et a/. BZR1 is a transcriptional repressor with dual roles in brassinosteroid homeostasis and growth responses. Science 307, 1634-1638 (2005).

19. Yin. Y. et a/ A new class of transcription factors mediates brassinosteroid-regulated gene expression in Arabidopsis. Ce11120, 249-259 (2005).

20. He, J. X., Gendron, J. M., Yang, Y., Li, J. & Wang, Z. Y. The GSK3-like kinase BIN2 phosphorylates and destabilizes BZR1, a positive regulator of the brassinosteroid signaling pathway in Arabidopsis. Proc Nat/Acad Sci USA 99, 10185-10190 (2002).

21. Ryu, H. et a/ Nucleocytoplasmic shuttling of BZR1 mediated by phosphorylation is essential in Arabidopsis brassinosteroid signaling. P/ant Cell 19, 2749-2762 (2007).

22. Vert, G. & Cho J. Downstream nuclear events in brassinosteroid signalling. Nature441. 96-100 (2006).

23. Peng, P., Yan, Z., Zhu, Y. & Li, J. Regulation of the Arabidopsis GSK3-like Kinase BRASS] NOSTEROID-INSENSITIVE 2 through Proteasome-Mediated Protein Degradation. Mo/Plant 1, 338-346 (2008).

24. Hu, C. D., Chinenov, Y. & Kerppola. T. K. Visualization of interactions among bZIP and Rel family proteins in living cells using bimolecular fluorescence complementation. Mot Ce/19, 789-798 (2002).

25. Benschop, J. J. et a; Quantitative phosphoproteomics of early elicitor signaling in Arabidopsis. Mot Cell Proteomics6. 1198-1214 (2007).

26. Sugiyama, N. et al Large-scale phosphorylation mapping reveals the extent of tyrosine phosphorylation in Arabidopsis. Mol Syst B/o/4, 193 (2008).

27. Hughes, K., Nikolakaki, E., Plyte, S. E., Tony, N. F. & Woodgett, J. R. Modulation of the glycogen synthase kinase-3 family by tyrosine phosphorylation. Embo J 12, 803-808 (1993).

28. Lochhead, P. A. et al. A chaperone-dependent GSK3beta transitional intermediate mediates activation-loop autophosphorylation. Mol Cett24, 627-633 (2006).

29. Kim, L., Harwood, A. & Kimmel, A. R. Receptor-dependent and tyrosine phosphatase-mediated inhibition of GSK3 regulates cell fate choice. Dev Cel/3, 523-532 (2002).

30. Shiu, S. H. et al. Comparative analysis of the receptor-like kinase family in Arabidopsis and rice. Plant Cell 16, 1220-1234 (2004).

31. Johnson, K. L. & Ingram, G. C. Sending the right signals: regulating receptor kinase activity. CurrOpinPlant Bio18, 648-656 (2005).

32. Zhou, A., Wang, H. Walker, J. C. & Li, J. BRL1, a leucine-rich repeat receptor-like protein kinase, is functionally redundant with BR11 in regulating Arabidopsis brassinosteroid signaling. Plant J40, 399-409 (2004).

33. Cano-Delgado, A. etal. BRL1 and BRL3 are novel brassinosteroid receptors that function in vascular differentiation in Arabidopsis. Development 131, 5341-5351 (2004).

34. Bhat, R. V. et a/ Regulation and localization of tyrosine216 phosphorylation of glycogen synthase kinase-3beta in cellular and animal models of neuronal degeneration. Proc Natl Aced Sci U S A 97, 11074-11079 (2000).

35. Kim. L. & Kimmel, A. R. GSK3 at the edge: regulation of developmental specification and cell polarization. Curr Drug Targets7, 1411-1419 (2006).

36. Jope, R. S. & Johnson. G. V. The glamour and gloom of glycogen synthase kinase-3. Trends Biochem Sci 29, 95-102 (2004).

37. Stadeli, R.. Hoffmans. R. & Basler, K. Transcription under the control of nuclear Arm/beta-catenin. Cun-Bio116. R378-385 (2006).

38. Zhan, X. L. et a/ Essential functions of protein tyrosine phosphatases PTP2 and PTP3 and RIM1 1 tyrosine phosphorylation in Saccharomyces cerevisiae meiosis and sporulation. Mol B/ol C611 11, 663-676 (2000).

39. Cole, A., Frame, S. & Cohen, P. Further evidence that the tyrosine phosphorylation of glycogen synthase kinase-3 (GSK3) in mammalian cells is an autophosphorylation event. Biochem J377, 249-255 (2004).

40. Kim, S. Lee. J.. Park, J. & Chung, J. BP75, bromodomain-containing M(r) 75,000 protein, binds dishevelled-1 and enhances Wnt signaling by inactivating glycogen synthase kinase-3 beta. Cancer Res63. 4792-4795 (2003).

41. MacKintosh, C. et a/ Further evidence that inhibitor-2 acts like a chaperone to fold PP1 into its native conformation. FEBSLett397. 235-238 (1996).

42. Puntoni. F. & Villa-Moruzzi, E. Phosphorylation of the inhibitor-2 of protein phosphatase-1 by cdc2-cyclin B and GSK3. Biochem Biophys Res Commun 207, 732-739 (1995).

43. Alonso, J. M. et a/ Genome-wide insertional mutagenesis of Arabidopsis thaliana. Science 301, 653-657 (2003).

44. Schwab, R., Ossowski, S., Riester, M., Warthmann, N. & Weigel, D. Highly specific gene silencing by artificial microRNAs in Arabidopsis. Plant Cell 18, 1121-1133 (2006).

45. Yamada, K. et al. Empirical analysis of transcriptional activity in the Arabidopsis genome. Science 302, 842-846 (2003).

46. Deng, Z. et al A proteomics study of brassinosteroid response in Arabidopsis. Mol Cell Proteomics 6, 2058-2071 (2007).

1-39. (canceled)

40. A method for treating a disease associated with abnormal cell growth comprising contacting a cell comprising glycogen synthase kinase 3 (GSK3) protein with a PP1 phosphatase or a homolog thereof in an amount effective to dephosphoylate the GSK3 protein.

41. The method of claim 40, wherein the GSK3 comprises the sequence of KQLVRGEXNXSYIXSRXY (SEQ ID NO: 34), wherein X is any amino acid.

42. The method of claim 41, wherein the first tyrosine residue is dephosphorylated.

43. The method of claim 40, wherein the GSK3 is GSK3α, GSK3β or BIN2.

44. The method of claim 43, wherein at least one tyrosine residue that corresponds to tyrosine 279 of GSK3α, tyrosine 216 of GSK3β, or tyrosine 200 of BIN2 is dephosphorylated.

45. The method of claim 40, wherein the PP1 phosphatase or homolog thereof is BSU1.

46. The method of claim 40, wherein the PP1 phosphatase or homolog thereof has at least 85% sequence identity an amino acid sequence selected from the group consisting of SEQ ID NO: 27, 28, 29, 30, 31, 32, and 33.

47. The method of claim 40, wherein the PP1 phosphatase or homolog thereof is introduced to the cell as a nucleic acid that endcodes the PP1 phosphatase or homolog thereof.

48. The method of claim 40, wherein the cell is ex vivo.

49. The method of claim 40, wherein the cell is a plant cell.

50. The method of claim 40, wherein the cell is an animal cell.

51. The method of claim 40, wherein the cell is a human cell.

52. The method of claim 40, wherein the PP1 phosphatase or a homolog thereof is an inactive mutant.

53. The method of claim 40, wherein the PP1 phosphatase or a homolog thereof is a constitutively active mutant.

54. The method of claim 47, further comprising contacting the cell with a PP1 1 agonist.

55. The method of claim 53, wherein the PP1 agonist is a brassinosteroid.

56. The method of claim 40, wherein the PP1 phosphatase or homolog thereof is an amino acid sequence selected from the group consisting of SEQ ID NO: 27, 28, 29, 30, 31, 32, and 33.


SEQUENCE LISTING (34 sequences)

SEQ ID NO: 1 - protein, 22 aa, Artificial
  Synthetic sequence
HKTMLPLPLIPSLLLSLSSAEF

SEQ ID NO: 2 - protein, 15 aa, Artificial
  Synthetic sequence
QPSLKRMKIQPSSQP

SEQ ID NO: 3 - protein, 7 aa, Artificial
  Synthetic sequence
MGSSKSK

SEQ ID NO: 4 - protein, 4 aa, Artificial
  Synthetic sequence
CXXX

SEQ ID NO: 5 - protein, 5 aa, Artificial
  Synthetic sequence
KXKXX

SEQ ID NO: 6 - protein, 7 aa, Artificial
  Synthetic sequence
PKKKRKV

SEQ ID NO: 7 - protein, 28 aa, Artificial
  Synthetic sequence
MSVLTPLLLRGLTGSARRLPVPRAKISL

SEQ ID NO: 8 - DNA, 32 bp, Artificial
  Synthetic sequence
gaagccaacatttctttcatctgctcacgatt

SEQ ID NO: 9 - DNA, 32 bp, Artificial
  Synthetic sequence
aagccaacatttctttcatctgctcacgattc

SEQ ID NO: 10 - DNA, 33 bp, Artificial
  Synthetic sequence
gaagccaacatttctgccatctgctcacgattc

SEQ ID NO: 11 - DNA, 33 bp, Artificial
  Synthetic sequence
gaatcgtgagcagatggcagaaatgttggcttc

SEQ ID NO: 12 - DNA, 37 bp, Artificial
  Synthetic sequence
cttttcttgaacttggttgcggagtatgtccctgaga

SEQ ID NO: 13 - DNA, 37 bp, Artificial
  Synthetic sequence
tctcagggacatactccgcaaccaagttcaagaaaag

SEQ ID NO: 14 - DNA, 37 bp, Artificial
  Synthetic sequence
gaacaccaacaagggaaaaaatcaaatgcatgaaccc

SEQ ID NO: 15 - DNA, 37 bp, Artificial
  Synthetic sequence
gggttcatgcatttgattttttcccttgttggtgttc

SEQ ID NO: 16 - DNA, 35 bp, Artificial
  Synthetic sequence
caatcaaagtcttcggcaatatccatggacaatac

SEQ ID NO: 17 - DNA, 35 bp, Artificial
  Synthetic sequence
gtattgtccatggatattgccgaagactttgattg

SEQ ID NO: 18 - DNA, 31 bp, Artificial
  Synthetic sequence
caccatggctcctgatcaatcttatcaatat

SEQ ID NO: 19 - DNA, 29 bp, Artificial
  Synthetic sequence
ttcacttgactcccctcgagctggagtag

SEQ ID NO: 20 - DNA, 31 bp, Artificial
  Synthetic sequence
caccatgggctcgaagccttggctacatcca

SEQ ID NO: 21 - DNA, 30 bp, Artificial
  Synthetic sequence
gatgtatgcaagcgagcttctgtcaaaatc

SEQ ID NO: 22 - DNA, 21 bp, Artificial
  Synthetic sequence
tattcatcaaaaaggcgcgtg

SEQ ID NO: 23 - DNA, 23 bp, Artificial
  Synthetic sequence
aagaggattcatggcggtctatg

SEQ ID NO: 24 - DNA, 22 bp, Artificial
  Synthetic sequence
gtattgttaagccgcccattgg

SEQ ID NO: 25 - DNA, 25 bp, Artificial
  Synthetic sequence
caaatccaaaaccctagaaaccgaa

SEQ ID NO: 26 - DNA, 22 bp, Artificial
  Synthetic sequence
atctcccgtaggacctgcactg

SEQ ID NO: 27 - protein, 793 aa, Arabidopsis thaliana
MAPDQSYQYPSPSYESIQTFYDTDEDWPGPRCGHTLTAVFVNNSHQLILFGGSTTAVANHNSSLPEISLDGVTNSVHSFDVLTRKWTRLNPIGDVPSPRACHAAALYGTLILIQGGIGPSGPSDGDVYMLDMTNNKWIKFLVGGETPSPRYGHVMDIAAQRWLVIFSGNNGNEILDDTWALDTRGPFSWDRLNPSGNQPSGRMYASGSSREDGIFLLCGGIDHSGVTLGDTYGLKMDSDNVWTPVPAVAPSPRYQHTAVFGGSKLHVIGGILNRARLIDGEAVVAVLDTETGEWVDTNQPETSASGANRQNQYQLMRRCHHAAASFGSHLYVHGGIREDVLLDDLLVAETSQSSSPEPEEDNPDNYMLLDDYLMDEPKPLSSEPEASSFIMRSTSEIAMDRLAEAHNLPTIENAFYDSAIEGYVPLQHGAETVGNRGGLVRTASLDQSTQDLHKKVISTLLRPKTWTPPANRDFFLSYLEVKHLCDEVEKIFMNEPTLLQLKVPIKVFGDIHGQYGDLMRLFHEYGHPSVEGDITHIDYLFLGDYVDRGQHSLEIIMLLFALKIEYPKNIHLIRGNHESLAMNRIYGFLTECEERMGESYGFEAWLKINQVFDYLPLAALLEKKVLCVHGGIGRAVTIEEIENIERPAFPDTGSMVLKDILWSDPTMNDTVLGIVDNARGEGVVSFGPDIVKAFLERNGLEMILRAHECVIDGFERFADGRLITVFSATNYCGTAQNAGAILVIGRDMVIYPKLIHPHPPPISSSEEDYTDKAWMQELNIEMPPTPARGESSE

SEQ ID NO: 28 - protein, 881 aa, Arabidopsis thaliana
MGSKPWLHPAPQYKTLETFWDDEDDAPGPRCAHTLTAVAATKTHGPRLILFGGATAIEGGSSSVPGIRLAGVTNTVHSYDILTRKWTRLKPAGEPPSPRAAHAAAAVGTMVVFQGGIGPAGHSTDDLYVLDMTNDKFKWHRVVVQGDGPGPRYGHVMDLVSQRYLVTVTGNDGKRALSDAWALDTAQKPYVWQRLNPDGDRPSARMYASGSARSDGMFLLCGGRDTLGAPLGDAYGLLMHRNGQWEWTLAPGVAPSPRYQHAAVFVGARLHVSGGVLRGGRVIDAEASVAVLDTAAGVWLDRNGQVTSARGSKGQIDQDPSFELMRRCRHGAASVGIRIYVHGGLRGDVLLDDFLVAENSTFQSDISSPLLASDRTQQSSTPRFSYAARPPSGSEPSFSMSEGLSLDENSLEKLTEASAAEAEVASSVWRAAQLGAGTLDEEPSTSDASSPIVESTTDGTANEGDVRLHPRAVVVAKETVGSLGGMVRQLSLDQFQNESRRMVPMNNSDVPQPTKKFTRQKSPQGLHKKVIAALLRPRNWKPPGNRKFFLDSYEVGELCYAAEQIFMHEQTVLQLKAPIKVFGDLHGQFGDLMRLFDEYGFPSTAGDITYIDYLFLGDYVDRGQHSLETITLLLALKIEYPENVHLIRGNHEAADINALFGFRLECIERMGENDGIWAWTRFNQLFNYLPLAALIENKIICMHGGIGRSISTVEQIEKIERPITMDAGSLVLMDLLWSDPTENDSIEGLRPNARGPGLVTFGPDRVTEFCKRNKLQLIIRAHECVMDGFERFAQGQLITLFSATNYCGTANNAGAILVVGRGLVIVPKLIHPLPPPILSPENSPEHSGDDAWMQELNIQRPPTPTRGRPQPDFDRSSLAYI

SEQ ID NO: 29 - protein, 1018 aa, Arabidopsis thaliana
MDEDSSMVADNDQDREFQSLDGGQSPSPMERETPQQMNDQSPPPEGGSVPTPPPSDPNPATSQQQAAAVVGQEQQPALVVGPRCAPTYSVVDAMMDKKEDGPGPRCGHTLTAVPAVGDEGTPGYIGPRLVLFGGATALEGNSGGTGTPTSAGSAGIRLAGATADVHCYDVLSNKWTRLTPFGEPPTPRAAHVATAVGTMVVIQGGIGPAGLSAEDLHVLDLTQQRPRWHRVVVQGPGPGPRYGHVMALVGQRYLMAIGGNDGKRPLADVWALDTAAKPYEWRKLEPEGEGPPPCMYATASARSDGLLLLCGGRDANSVPLASAYGLAKHRDGRWEWAIAPGVSPSSRYQHAAVFVNARLHVSGGALGGGRMVEDSSSVAVLDTAAGVWCDTKSVVTSPRTGRYSADAAGGDASVELTRRCRHAAAAVGDLIFIYGGLRGGVLLDDLLVAEDLAAAETTYAASHAAAAAATNSPPGRLPGRYGFSDERNRELSESAADGAVVLGSPVAPPVNGDMHTDISPENALLPGTRRTNKGVEYLVEASAAEAEAISATLAAAKARQVNGEVELPDRDCGAEATPSGKPTFSLIKPDSMGSMSVTPAGIRLHHRAVVVAAETGGALGGMVRQLSIDQFENEGRRVSYGTPESATAARKLLDRQMSINSVPKKVIAHLLKPRGWKPPVRRQFFLDCNEIADLCDSAERIFASEPTVLQLKAPIKIFGDLHGQFGDLMRLFDEYGSPSTAGDISYIDYLFLGDYVDRGQHSLETISLLLALKVEYQHNVHLIRGNHEAADINALFGFRIECIERMGERDGIWVWHRINRLFNWLPLAASIEKKIICMHGGIGRSINHVEQIENIQRPITMEAGSIVLMDLLWSDPTENDSVEGLRPNARGPGLVTFGPDRVMEFCNNNDLQLIVRAHECVMDGFERFAQGHLITLFSATNYCGTANNAGAILVLGRDLVVVPKLIHPLPPALSSPETSPERHIEDTWMQELNANRPATPTRGRPQNSNDRGGSLAWM

SEQ ID NO: 30 - protein, 1006 aa, Arabidopsis thaliana
MDLDSSMVPENDQDPIATSENQSPMEEKEEASEQQTGSESESASLTPSLPPPSQQQQQQQQQPQVTAVVGPRCAPTYSVVNAIIEKKEDGPGPRCGHTLTAVPAVGEEGTSSYIGPRLILFGGATALEGNSGGTGTPTSAGSAGIRLAGATADVHCYDVLSNKWSRLTPYGEPPSPRAAHVATAVGTMVVIQGGIGPAGLSAEDLHVLDLTQQRPRWHRVVVQGPGPGPRYGHVMALVGQRYLMAIGGNDGKRPLADVWALDTAAKPYEWRKLEPEGEGPPPCMYATASARSDGLLLLCGGRDANSVPLASAYGLAKHRDGRWEWAIAPGVSPSARYQHAAVFVNARLHVSGGALGGGRMVEDSSSVAVLDTAAGVWCDTKSVVTSPRTGRYSADAAGGDASVELTRRCRHAAAAVGDLIFIYGGLRGGVLLDDLLVAEDLAAAETTSAASHAAAAAAATNTPPGRSPGRYGFSDERTGELPESAPDAVVLGSPVAPPVNGDMYTDISTENAMVPGIRRTSKGVEYLVEASAAEAEAISATLAAAKARQVNGEVELPDRDRGAEATPSGKPSLSLIKPDSAVPNSVIPAGVRLHHRAVVVAAETGGALGGMVRQLSIDQFENEGRRVSYGTPESATAARKLLDRQMSINSVPKKVVAHLLKPRGWKPPVRRQFFLDCNEIADLCDSAERIFSSEPTVLQLKAPIKIFGDLHGQFGDLMRLFDEYGSPSTAGDISYIDYLFLGDYVDRGQHSLETITLLLALKVEYQHNVHLIRGNHEAADINALFGFRIECIERMGERDGIWVWHRINRLFNWLPLAALIEKKIICMHGGIGRSINHVEQIENIQRPITMEAGSIVLMDLLWSDPTENDSVEGLRPNARGPGLVTFGPDRVMEFCNNNDLQLIVRAHECVMDGFERFAQGHLITLFSATNYCGTANNAGAILVLGRDLVVVPKLIHPLPPAITSPETSPERHIEDTWMQELNVNRPPTPTRGRPQNPNDRGSLAWI

SEQ ID NO: 31 - protein, 323 aa, Homo sapiens
MADLDKLNIDSIIQRLLEVRGSKPGKNVQLQENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVLGWGENDRGVSFTFGAEVVAKFLHKHDLDLICRAHQVVEDGYEFFAKRQLVTLFSAPNYCGEFDNAGAMMSVDETLMCSFQILKPAEKKKPNATRPVTPPRGMITKQAKK

SEQ ID NO: 32 - protein, 327 aa, Homo sapiens
MADGELNVDSLITRLLEVRGCRPGKIVQMTEAEVRGLCIKSREIFLSQPILLELEAPLKICGDIHGQYTDLLRLFEYGGFPPEANYLFLGDYVDRGKQSLETICLLLAYKIKYPENFFLLRGNHECASINRIYGFYDECKRRFNIKLWKTFTDCFNCLPIAAIVDEKIFCCHGGLSPDLQSMEQIRRIMRPTDVPDTGLLCDLLWSDPDKDVQGWGENDRGVSFTFGADVVSKFLNRHDLDLICRAHQVVEDGYEFFAKRQLVTLFSAPNYCGEFDNAGGMMSVDETLMCSFQILKPSEKKAKYQYGGLNSGRPVTPPRTANPPKKR

SEQ ID NO: 33 - protein, 330 aa, Homo sapiens
MSDSEKLNLDSIIGRLLEVQGSRPGKNVQLTENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVQGWGENDRGVSFTFGAEVVAKFLHKHDLDLICRAHQVVEDGYEFFAKRQLVTLFSAPNYCGEFDNAGAMMSVDETLMCSFQILKPADKNKGKYGQFSGLNPGGRPITPPRNSAKAKK

SEQ ID NO: 34 - protein, 18 aa, Artificial
  Synthetic sequence
KQLVRGEXNXSYIXSRXY